SEQUENCE LISTING (115 sequences)

SEQ ID NO: 1 - protein, 465 aa, Oryza sativa
MDPSPAVDPSPAVDPSPAAETRRRATGKGGKQRGGKQLGLKRPPPISVPATPPPAATSSSPAAPTAIPPRPPQSSPIFVPDSPNPSPAAPTSSLASGTSTARPPQPQGGGWGPTSTISPNFASFFGNQQDPNSCLVRGYPPGGFVNFIQQNCPPQPQQQGENFHFVGHNMGFNPISPQPPSAYGTPTPQATNQGTSTNIMIDEEDNNDDSRAAKKRWTHEEEERLASAWLNASKDSIHGNDKKGDTFWKEVTDEFNKKGNGKRRREINQLKVHWSRLKSAISEFNDYWSTVTQMHTSGYSDDMLEKEAQRLYANRFGKPFALVHWWKILKREPKWCAQFEKRKRKSEMDAVPEQQKRPIGREAAKSERKRKRKKENVMEGIVLLGDNVQKIIKVTQDRKLEREKVTEAQIHISNVNLKAAEQQKEAKMFEVYNSLLTQDTSNMSEEQKARRDKALQKLEEKLFAD

SEQ ID NO: 2 - DNA, 1398 bp, Oryza sativa
  source 1..1398: mol_type = genomic DNA
atggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactag

SEQ ID NO: 3 - protein, 482 aa, Oryza sativa
MQSLAISLLLSETHSLFSHTKTSSLLSLLFLSSSKMSEQNTDGSQVPVNLLDEFLAEDEIIDDLLTEATVVVQSTIEGLQNEASDHRHHPRKHIKRPREEAHQQLVNDYFSENPLYPSKIFRRRFRMSRPLFLRIVEALGQWSVYFTQRVDAVNRKGLSPLQKCTAAIRQLATGSGADELDEYLKIGETTAMEAMKNFVKGLQDVFGERYLRRPTMEDTERLLQLGEKRGFPGMFGSIDCMHWHWERCPVAWKGQFTRGDQKVPTLILEAVASHDLWIWHAFFGAAGSNNDINVLNQSTVFIKELKGQAPRVQYMVNGNQYNTGYFLADGIYPEWAVFVKSIRLPNTEKEKLYADMQEGARKDIERAFGVLQRRFCILKRPARLYDRGVLRDVVLACIILHNMIVEDEKETRIIEEDADANVPPSSSTVQEPEFSPEQNTPFDRVLEKDISIRDRAAHNRLKKDLVEHIWNKFGGAAHRTGN

SEQ ID NO: 4 - DNA, 1446 bp, Oryza sativa
  source 1..1446: mol_type = genomic DNA
atgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaat

SEQ ID NO: 5 - protein, 1399 aa, synthetic construct
APKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 6 - DNA, 4200 bp, synthetic construct
  source 1..4200: mol_type = other DNA
gctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtga

SEQ ID NO: 7 - DNA, 12 bp, Oryza sativa
  source 1..12: mol_type = genomic DNA
ggccagtcacaa

SEQ ID NO: 8 - DNA, 12 bp, Oryza sativa
  source 1..12: mol_type = genomic DNA
ttgtgactggcc

SEQ ID NO: 9 - DNA, 15 bp, synthetic construct
  source 1..15: mol_type = other DNA
ttaggccagtcacaa

SEQ ID NO: 10 - DNA, 15 bp, synthetic construct
  source 1..15: mol_type = other DNA
ttgtgactggcctta

SEQ ID NO: 11 - DNA, 68 bp, Arabidopsis thaliana
  source 1..68: mol_type = genomic DNA
ccatcttgggcctcaacataagcctgaccgccgaccatggctggcaaaagtccaatagcaaactttat

SEQ ID NO: 12 - DNA, 23 bp, synthetic construct
  source 1..23: mol_type = other DNA
cataagcctgaggccagtcacaa

SEQ ID NO: 13 - DNA, 38 bp, synthetic construct
  source 1..38: mol_type = other DNA
ttgtgactggccttagcgccgaccatggctggcaaaag

SEQ ID NO: 14 - DNA, 23 bp, synthetic construct
  source 1..23: mol_type = other DNA
cataagcctgaggccagtcacaa

SEQ ID NO: 15 - DNA, 38 bp, synthetic construct; identical to SEQ ID NO: 13
  source 1..38: mol_type = other DNA

SEQ ID NO: 16 - DNA, 23 bp, synthetic construct
  source 1..23: mol_type = other DNA
cataagcctgaggccagtcacaa

SEQ ID NO: 17 - DNA, 34 bp, synthetic construct
  source 1..34: mol_type = other DNA
ttgtgactggcctgccgaccatggctggcaaaag

SEQ ID NO: 18 - DNA, 27 bp, synthetic construct
  source 1..27: mol_type = other DNA
cataagcctgacttaggccagtcacaa

SEQ ID NO: 19 - DNA, 34 bp, synthetic construct; identical to SEQ ID NO: 17
  source 1..34: mol_type = other DNA

SEQ ID NO: 20 - DNA, 27 bp, synthetic construct
  source 1..27: mol_type = other DNA
cataagcctgacttaggccagtcacaa

SEQ ID NO: 21 - DNA, 33 bp, synthetic construct
  source 1..33: mol_type = other DNA
ttgtgactggccgccgaccatggctggcaaaag

SEQ ID NO: 22 - DNA, 25 bp, synthetic construct
  source 1..25: mol_type = other DNA
cataagcctgacaggccagtcacaa

SEQ ID NO: 23 - DNA, 33 bp, synthetic construct; identical to SEQ ID NO: 21
  source 1..33: mol_type = other DNA

SEQ ID NO: 24 - DNA, 25 bp, synthetic construct
  source 1..25: mol_type = other DNA
cataagcctgacaggccagtcacaa

SEQ ID NO: 25 - DNA, 36 bp, synthetic construct
  source 1..36: mol_type = other DNA
ttgtgactggccttaaccgaccatggctggcaaaag

SEQ ID NO: 26 - DNA, 28 bp, synthetic construct
  source 1..28: mol_type = other DNA
cataagcctgacgttaggccagtcacaa

SEQ ID NO: 27 - DNA, 37 bp, synthetic construct
  source 1..37: mol_type = other DNA
ttgtgactggccttacgccgaccatggctggcaaaag

SEQ ID NO: 28 - DNA, 17 bp, synthetic construct
  source 1..17: mol_type = other DNA
cataagcctgactgtgt

SEQ ID NO: 29 - DNA, 33 bp, synthetic construct; identical to SEQ ID NO: 21
  source 1..33: mol_type = other DNA

SEQ ID NO: 30 - DNA, 25 bp, synthetic construct
  source 1..25: mol_type = other DNA
cataagcctgataggccagtcacaa

SEQ ID NO: 31 - DNA, 27 bp, synthetic construct
  source 1..27: mol_type = other DNA
ttgtgactggcctatggctggcaaaag

SEQ ID NO: 32 - DNA, 25 bp, synthetic construct
  source 1..25: mol_type = other DNA
cataagcctgataggccagtcacaa

SEQ ID NO: 33 - DNA, 27 bp, synthetic construct
  source 1..27: mol_type = other DNA
ttgtgactggcctatggctggcaaaag

SEQ ID NO: 34 - DNA, 25 bp, synthetic construct
  source 1..25: mol_type = other DNA
cataagcctgataggccagtcacaa

SEQ ID NO: 35 - DNA, 27 bp, synthetic construct
  source 1..27: mol_type = other DNA
ttgtgactggcctatggctggcaaaag

SEQ ID NO: 36 - DNA, 25 bp, synthetic construct
  source 1..25: mol_type = other DNA
cataagcctgataggccagtcacaa

SEQ ID NO: 37 - DNA, 27 bp, synthetic construct
  source 1..27: mol_type = other DNA
ttgtgactggcctatggctggcaaaag

SEQ ID NO: 38 - DNA, 27 bp, synthetic construct
  source 1..27: mol_type = other DNA
ttgtgactggcctatggctggcaaaag

SEQ ID NO: 39 - DNA, 27 bp, synthetic construct
  source 1..27: mol_type = other DNA
ttgtgactggcctatggctggcaaaag

SEQ ID NO: 40 - DNA, 27 bp, synthetic construct
  source 1..27: mol_type = other DNA
cataagcctgactaaggccagtcacaa

SEQ ID NO: 41 - DNA, 36 bp, synthetic construct
  source 1..36: mol_type = other DNA
ttgtgactggccttcgccgaccatggctggcaaaag

SEQ ID NO: 42 - DNA, 24 bp, synthetic construct
  source 1..24: mol_type = other DNA
cataagcctgaaggccagtcacaa

SEQ ID NO: 43 - DNA, 36 bp, synthetic construct; identical to SEQ ID NO: 41
  source 1..36: mol_type = other DNA

SEQ ID NO: 44 - DNA, 24 bp, synthetic construct
  source 1..24: mol_type = other DNA
cataagcctgaaggccagtcacaa

SEQ ID NO: 45 - DNA, 36 bp, synthetic construct; identical to SEQ ID NO: 41
  source 1..36: mol_type = other DNA

SEQ ID NO: 46 - DNA, 28 bp, synthetic construct
  source 1..28: mol_type = other DNA
cataagcctgacttaaggccagtcacaa

SEQ ID NO: 47 - DNA, 36 bp, synthetic construct; identical to SEQ ID NO: 41
  source 1..36: mol_type = other DNA

SEQ ID NO: 48 - DNA, 31 bp, synthetic construct
  source 1..31: mol_type = other DNA
caacataagcctgacaggccagtcacaatgg

SEQ ID NO: 49 - DNA, 15 bp, synthetic construct
  source 1..15: mol_type = other DNA
ccattgtgactggcc

SEQ ID NO: 50 - DNA, 14 bp, synthetic construct
  source 1..14: mol_type = other DNA
gccgaccatggctg

SEQ ID NO: 51 - DNA, 15 bp, synthetic construct
  source 1..15: mol_type = other DNA
caacataagcctgac

SEQ ID NO: 52 - DNA, 15 bp, synthetic construct
  source 1..15: mol_type = other DNA
ggccagtcacaatgg

SEQ ID NO: 53 - DNA, 30 bp, synthetic construct
  source 1..30: mol_type = other DNA
ccattgtgactggcccgccgaccatggctg

SEQ ID NO: 54 - DNA, 32 bp, synthetic construct
  source 1..32: mol_type = other DNA
ccgttgtttccacgtaaggccagtcacaatgg

SEQ ID NO: 55 - DNA, 30 bp, synthetic construct
  source 1..30: mol_type = other DNA
ccattgtgactggccatcttcggccatgaa

SEQ ID NO: 56 - DNA, 15 bp, synthetic construct
  source 1..15: mol_type = other DNA
ccgttgtttccacgt

SEQ ID NO: 57 - DNA, 15 bp, synthetic construct
  source 1..15: mol_type = other DNA
ggccagtcacaatgg

SEQ ID NO: 58 - DNA, 29 bp, synthetic construct
  source 1..29: mol_type = other DNA
ccatgtgactggccatcttcggccatgaa

SEQ ID NO: 59 - DNA, 14 bp, synthetic construct
  source 1..14: mol_type = other DNA
tacaggagtagttc

SEQ ID NO: 60 - DNA, 14 bp, synthetic construct
  source 1..14: mol_type = other DNA
gccagtcacaatgg

SEQ ID NO: 61 - DNA, 30 bp, synthetic construct
  source 1..30: mol_type = other DNA
ccattgtgactggcctcgtggccttagtaa

SEQ ID NO: 62 - DNA, 30 bp, synthetic construct
  source 1..30: mol_type = other DNA
tacaggagtagttcaggccagtcacaatgg

SEQ ID NO: 63 - DNA, 30 bp, synthetic construct; identical to SEQ ID NO: 61
  source 1..30: mol_type = other DNA

SEQ ID NO: 64 - protein, 5 aa, synthetic construct
GSSSS

SEQ ID NO: 65 - DNA, 20 bp, synthetic construct
  source 1..20: mol_type = other DNA
gccagccatggtcggcggtc

SEQ ID NO: 66 - DNA, 20 bp, synthetic construct
  source 1..20: mol_type = other DNA
gcttcatggccgaagatacg

SEQ ID NO: 67 - DNA, 20 bp, synthetic construct
  source 1..20: mol_type = other DNA
gttacaggagtagttcatcg

SEQ ID NO: 68 - protein, 8 aa, synthetic construct
GGSGGGSG

SEQ ID NO: 69 - protein, 5 aa, synthetic construct
GGGGS

SEQ ID NO: 70 - protein, 12 aa, synthetic construct
AEAAAKEAAAKA

SEQ ID NO: 71 - protein, 17 aa, synthetic construct
AEAAAKEAAAKEAAAKA

SEQ ID NO: 72 - protein, 5 aa, synthetic construct
PAPAP

SEQ ID NO: 73 - protein, 8 aa, synthetic construct
GIHGVPAA

SEQ ID NO: 74 - DNA, 23463 bp, synthetic construct
  source 1..23463: mol_type = other DNA
gtttacccgccaatatatcctgtcaaacactgatagtttttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctgggaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgccagccatggtcggcggtcgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 75 - DNA, 21092 bp, synthetic construct
  source 1..21092: mol_type = other DNA
gtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgccagccatggtcggcggtcgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttag

SEQ ID NO: 76 - protein, 5 aa, synthetic construct
EAAAK

SEQ ID NO: 77 - protein, 10 aa, synthetic construct
EAAAKEAAAK

SEQ ID NO: 78 - protein, 15 aa, synthetic construct
EAAAKEAAAKEAAAK

SEQ ID NO: 79 - protein, 20 aa, synthetic construct
EAAAKEAAAKEAAAKEAAAK

SEQ ID NO: 80 - DNA, 20 bp, synthetic construct
  source 1..20: mol_type = other DNA
ggaactgacacacgacatga

SEQ ID NO: 81 - DNA, 444 bp, synthetic construct
  source 1..444: mol_type = other DNA
ggccagtcacaatggctagtgtcattgcacggctacccaaaatattataccatcttctctcaaatgaaatcttttatgaaacaatccccacagtggaggggtttcttgaacgttccaagactaagcaaagcatttaattgatacaagttcgcgaagattcatttgtacccaaaatccggcgcggcgcgggagaatgttctggaaggtcgcacggcggaggcggacgcaagagatccggtgaatgttcaagaatcggcctcaacgggggtttcactctgttaccgaggaactttctggaaacgacgctgacgagtttcaccaggatgaaactctttccagaaagttctctctcatccccatttcatgcaaataatcattttttattcagtcttacccctattaaatgtgcatgacacaccagtgaaacccccattgtgactggcc

SEQ ID NO: 82 - DNA, 13 bp, synthetic construct
  source 1..13: mol_type = other DNA
ttcttgaacgttc

SEQ ID NO: 83 - DNA, 13 bp, synthetic construct
  source 1..13: mol_type = other DNA
ttcgcgaagattc

SEQ ID NO: 84 - DNA, 13 bp, synthetic construct
  source 1..13: mol_type = other DNA
ttccagaacattc

SEQ ID NO: 85 - DNA, 13 bp, synthetic construct
  source 1..13: mol_type = other DNA
ttcttgaacattc

SEQ ID NO: 86 - DNA, 13 bp, synthetic construct
  source 1..13: mol_type = other DNA
ttccagaaagttc

SEQ ID NO: 87 - DNA, 13 bp, synthetic construct
  source 1..13: mol_type = other DNA
ttccagaaagttc

SEQ ID NO: 88 - protein, 9 aa, synthetic construct
GGSGGSGGS

SEQ ID NO: 89 - DNA, 22510 bp, synthetic construct
  source 1..22510: mol_type = other DNA
gtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattaattaattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgaggcgcgcctctagatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 90 - DNA, 14122 bp, synthetic construct
  source 1..14122: mol_type = other DNA
tggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctggggacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 91 - DNA, 21085 bp, synthetic construct
  source 1..21085: mol_type = other DNA
gtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 92 - DNA, 21560 bp, synthetic construct
  source 1..21560: mol_type = other DNA
gtttacccgccaatatatcctgtcaaacactgatagttttgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 93 - DNA, 21585 bp, synthetic construct
  source 1..21585: mol_type = other DNA
gtttacccgccaatatatcctgtcaaacactgatagtttcacgtgatctccttggatcctctagattaggccagtcacaatggctagtgtcattgcacggctacccaaaatattataccatcttctctcaaatgaaatcttttatgaaacaatccccacagtggaggggtttcttgaacgttccaagactaagcaaagcatttaattgatacaagttcgcgaagattcatttgtacccaaaatccggcgcggcgcgggagaatgttctggaaggtcgcacggcggaggcggacgcaagagatccggtgaatgttcaagaatcggcctcaacgggggtttcactctgttaccgaggaactttctggaaacgacgctgacgagtttcaccaggatgaaactctttccagaaagttctctctcatccccatttcatgcaaataatcattttttattcagtcttacccctattaaatgtgcatgacacaccagtgaaacccccattgtgactggccttatctagagtcccccatactaggcctaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 94 - DNA, 23380 bp, synthetic construct
  source 1..23380: mol_type = other DNA
ggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgagcctagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgactctagtgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggaactgacacacgacatgagttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatggttatactaggagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattaattaattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctagtgatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgaggcgcgcctctagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgctagaggatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggt

SEQ ID NO: 95 - DNA, 23836 bp, synthetic construct
  source 1..23836: mol_type = other DNA
tggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgagcctagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgactctagcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctgggaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggaactgacacacgacatgagttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctagaggatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 96 - DNA, 430 bp, synthetic construct
  source 1..430: mol_type = other DNA
ggccagtcacaatgggggtttcactggtgtgtcatgcacatttaataggggtaagactgaataaaaaatgattatttgcatgaaatggggatgagagagaaggaaagagtttcatcctggtgaaactcgtcagcgtcgtttccaagtcctcggtaacagagtgaaacccccgttgaggccgattcgtttcattcaccggatctcttgcgtccgcctccgccgtgcgacctccgcattctcccgcgccgcgccggattttgggtacaaatgatcccagcaacttgtatcaattaaatgctttgcttagtcttggaaacgtcaaagtgaaacccctccactgtggggattgtttcataaaagatttcatttgagagaagatggtataatattttgggtagccgtgcaatgacactagccattgtgactggcc

SEQ ID NO: 97 - DNA, 1570 bp, synthetic construct
  source 1..1570: mol_type = other DNA
taaggccagtcacaatgggggtttcactggtgtgtcatgcacatttaataggggtaagactgaataaaaaatgattatttgcatgaaatggggatgagagagaaggaaagagtttcatcctggtgaaactcgtcagcgtcgtttccaagtcctcggtaacagagtgaaacccccgttgaggccgattcgtttcattcaccggatctcttgcgtccgcctccgccgtgcgacctccgcattctcccgcgccgcgccgatcgatcatgagcggagaattaagggagtcacgttatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatctgatgacccctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaggattttgggtacaaatgatcccagcaacttgtatcaattaaatgctttgcttagtcttggaaacgtcaaagtgaaacccctccactgtggggattgtttcataaaagatttcatttgagagaagatggtataatattttgggtagccgtgcaatgacactagccattgtgactggcctaa

SEQ ID NO: 98 - DNA, 1008 bp, synthetic construct
  source 1..1008: mol_type = other DNA
taaggccagtcacaatgggggtttcactggtgtgtcatgcacatttaataggggtaagactgaataaaaaatgattatttgcatgaaatggggatgagagagaaggaaagagtttcatcctggtgaaactcgtcagcgtcgtttccaagtcctcggtaacagagtgaaacccccgttgaggccgattcgtttcattcaccggatctcttgcgtccgcctccgccgtgcgacctccgcattctcccgcgccgcgccgatcgaggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatctgatgaggattttgggtacaaatgatcccagcaacttgtatcaattaaatgctttgcttagtcttggaaacgtcaaagtgaaacccctccactgtggggattgtttcataaaagatttcatttgagagaagatggtataatattttgggtagccgtgcaatgacactagccattgtgactggcctaa

SEQ ID NO: 99 - DNA, 1303 bp, synthetic construct
  source 1..1303: mol_type = other DNA
atggcgaatgctagagcagcttgagcttggatcagattgtcgtttcccgccttcagtttgggggactctagataaggccagtcacaatgggggtttcactggtgtgtcatcgatcatgagcggagaattaagggagtcacgttatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatctgatgacccctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagccgtgcaatgacactagccattgtgactggcctaatctagaggatccaaggagataagctg

SEQ ID NO: 100 - DNA, 3910 bp, synthetic construct
  source 1..3910: mol_type = other DNA
ctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcagga

SEQ ID NO: 101 - DNA, 3143 bp, synthetic construct
  source 1..3143: mol_type = other DNA
agcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattaattaattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagat

SEQ ID NO: 102 - DNA, 5639 bp, synthetic construct
  source 1..5639: mol_type = other DNA
gatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgaggcgcgcctctagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 103 - DNA, 712 bp, synthetic construct
  source 1..712: mol_type = other DNA
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaata

SEQ ID NO: 104 - DNA, 7268 bp, synthetic construct
  source 1..7268: mol_type = other DNA
agcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattacggcggaggtggttctggcggtggaggttcaggcggtggtggaagtatggcgccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 105 - DNA, 712 bp, synthetic construct
  source 1..712: mol_type = other DNA
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggaactgacacacgacatgagttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaata

SEQ ID NO: 106 - DNA, 5664 bp, synthetic construct
  source 1..5664: mol_type = other DNA
atgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtga

SEQ ID NO: 107 - DNA, 5697 bp, synthetic construct
  source 1..5697: mol_type = other DNA
atgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattacggcggaggtggttctggcggtggaggttcaggcggtggtggaagtatggcgccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtga

SEQ ID NO: 108 - DNA, 253 bp, synthetic construct
  source 1..253: mol_type = other DNA
ggccagtcacaatgggggtttcactggtgtgtcatgcacatttaataggggtaagactgaataaaaaatgattatttgcatgaaatggggatgagagagaaggaaagagtttcatcctggtgaaactcgtcagcgtcgtttccaagtcctcggtaacagagtgaaacccccgttgaggccgattcgtttcattcaccggatctcttgcgtccgcctccgccgtgcgacctccgcattctcccgcgccgcgccg

SEQ ID NO: 109 - DNA, 178 bp, synthetic construct
  source 1..178: mol_type = other DNA
ggattttgggtacaaatgatcccagcaacttgtatcaattaaatgctttgcttagtcttggaaacgtcaaagtgaaacccctccactgtggggattgtttcataaaagatttcatttgagagaagatggtataatattttgggtagccgtgcaatgacactagccattgtgactggcc

SEQ ID NO: 110 - DNA, 5625 bp, synthetic construct
  source 1..5625: mol_type = other DNA
atgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttgataagaagtactctatcggactcgctatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcaaatcctcaaagagcaccctgttgagaacactcagctccagaatgagaagctctacctctactacctccagaacggaagggatatgtatgtggatcaagagttggatatcaacaggctctctgattacgatgttgatgctatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgatacgcgtgcggatcctaagaagaagaggaaggtttga

SEQ ID NO: 111 - DNA, 33 bp, synthetic construct
  source 1..33: mol_type = other DNA
ggccagtcacaatgggggtttcactggtgtgtc

SEQ ID NO: 112 - DNA, 33 bp, synthetic construct
  source 1..33: mol_type = other DNA
gccgtgcaatgacactagccattgtgactggcc

SEQ ID NO: 113 - DNA, 20 bp, synthetic construct
  source 1..20: mol_type = other DNA
gttacaggagtagttcatcg

SEQ ID NO: 114 - DNA, 1286 bp, synthetic construct
  source 1..1286: mol_type = other DNA
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatattaatccaaactactgcagcctgacagacaaatgaggatgcaaacaattttaaagtttatctaacgctagctgttttgtttcttctctctggtgcaccaacgacggcgttttctcaatcataaagaggcttgttttacttaaggccaataatgttgatggatcgaaagaagagggcttttaataaacgagcccgtttaagctgtaaacgatgtcaaaaacatcccacatcgttcagttgaaaatagaagctctgtttatatattggtagagtcgactaagagattgccgagtcgaggagacaacgggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaata

SEQ ID NO: 115 - DNA, 7318 bp, synthetic construct
  source 1..7318: mol_type = other DNA
agcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttgataagaagtactctatcggactcgctatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcaaatcctcaaagagcaccctgttgagaacactcagctccagaatgagaagctctacctctactacctccagaacggaagggatatgtatgtggatcaagagttggatatcaacaggctctctgattacgatgttgatgctatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgatacgcgtgcggatcctaagaagaagaggaaggtttgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagat

(19) United States
(12) Patent Application Publication
(10) Pub. No.: US 2026/0286372 A1
(43) Pub. Date: Sep. 24, 2026

(54) TARGETED INSERTION VIA TRANSPOSITION

(71) Applicant: Donald Danforth Plant Science Center, St. Louis, MO (US)

(72) Inventors: R. Keith Slotkin, St Louis, MO (US); Peng Liu, St. Louis, MO (US)

(21) Appl. No.: 19/126,724

(22) PCT Filed: Nov. 6, 2023
(86) PCT No.: PCT/US2023/078837

§ 371 Date: May 2, 2025

Related U.S. Application Data
(60) Provisional application No. 63/382,355, filed on Nov. 4, 2022.

(51) Int. Cl. C12N 15/82; C12N 9/12; C12N 9/22; C12N 15/11

(52) U.S. Cl. CPC C12N 15/8209; C12N 9/1241; C12N 9/226; C12N 15/111; C12N 2310/20; C12N 2800/90

(57) ABSTRACT

The present disclosure describes systems and methods for accurately inserting a donor polynucleotide into a target nucleic acid locus. The systems include a programmable targeting nuclease, a transposase, and a donor polynucleotide flanked by transposition sequences compatible with the transposase.

### CROSS REFERENCE TO RELATED APPLICATIONS

This application claims priority from Provisional Application No. 63/382,355, filed Nov. 4, 2022, the entire contents of which are hereby incorporated by reference.

### SEQUENCE LISTING

This application contains a Sequence Listing that has been submitted in .XML format via PatentCenter and is hereby incorporated by reference in its entirety. The XML is named “077875-768591 Sequence Listing.xml” and is 361 kilobytes in size.

### FIELD OF THE INVENTION

The present disclosure provides systems and methods of accurately inserting a donor polynucleotide into a target nucleic acid locus.

### BACKGROUND OF THE INVENTION

Genome editing is a revolutionary technology that promises the ability to improve or overcome current deficiencies in the genetic code as well as to introduce novel functionality. However, some applications of the technology do not always generate completely reliable results. For instance, transgene integration into or near genes can generate new mutations or alter the regulation of nearby genes, while insertions into heterochromatic regions are often not permissive to the desired high levels of transgene expression or do not provide stable expression over multiple generations. Further, in most instances, when performing transgenesis, the transgene frequently inserts into the nuclear genome in a random location. This can lead to new mutations at the insertion locus and at unintended insertion points, gene silencing, and general inconsistencies in experiments or products. For instance, in plants, where the frequency of homologous recombination is less than 1%, efficient and accurate insertion of transgenes is possible only in theory and is often associated with uncontrolled deletions of neighboring regions, as well as rearrangement of the transgene sequences. In fact, in a typical scenario, it simply is not possible to obtain the optimal, desired change. Additionally, although recently developed tools such as CRISPR systems have allowed biologists to target random genetic modifications to specific regions of genomes, accurate nucleic insertions in target loci is still a major challenge. In plants, this is because Homologous Recombination (HR) and Homology-Directed Repair (HDR) of donor sequences into the targeted locus occurs at a very low frequency.

Therefore, a long-felt need exists for improved and effective means of inserting polynucleotides into a user-defined location in the genome, especially in organisms where the frequency of HR and HDR are low, including plants.

### SUMMARY OF THE INVENTION

One aspect of the instant disclosure encompasses an engineered nucleic acid modification system for generating a genetically modified cell. The system comprises (a) a donor polynucleotide comprising a first and second mPing miniature inverted-repeat transposable element (MITE) transposition sequences; (b) one or more nucleic acid constructs for expressing a tranposase comprising a promoter operably linked to a nucleic acid sequence encoding the Pong ORF1 protein and a promoter operably linked to a nucleic acid sequence encoding the Pong ORF2 protein; and (c) a nucleic acid expression construct for expressing a programmable targeting system, wherein the expression construct comprises a promoter operably linked to a nucleic acid sequence encoding the programmable targeting system. The programmable targeting system is programmed to target the transposase and the donor polynucleotide to a target nucleic acid locus in the cell, to introduce a cut in the target nucleic acid locus, or both, thereby accomplishing insertion of the donor polynucleotide at the target nucleic acid locus to generate a genetically modified cell comprising the donor polynucleotide inserted at the target nucleic acid locus. The engineered system can further comprise a reporter nucleic acid construct for expressing a reporter, wherein the reporter nucleic acid construct comprises a promoter operably linked to a polynucleotide sequence encoding the reporter, wherein the donor polynucleotide is inserted in the reporter nucleic acid construct thereby inactivating expression of the reporter, and wherein expression of the reporter is activated by excision of the inserted donor polynucleotide from the reporter nucleic acid construct by the transposase. In some aspects, the cell is a plant cell, a plant or part thereof, or seed.

The first transposition sequence can comprise a nucleic acid sequence comprising at least about 75% or more, at least about 85% or more, at least about 95% or more, or 100% sequence identity with SEQ ID NO: 7, SEQ ID NO: 111, or SEQ ID NO: 108. The second transposition sequence can comprise a nucleic acid sequence comprising at least about 75% or more, at least about 85% or more, at least about 95% or more, or 100% sequence identity with SEQ ID NO: 8, SEQ ID NO: 112, or SEQ ID NO: 109.

In some aspects, the Pong ORF1 protein comprises an amino acid sequence comprising at least about 75% or more, at least about 85% or more, at least about 95% or more, or 100% sequence identity with the amino acid sequence of SEQ ID NO: 1. In some aspects, a nucleic acid sequence encoding the Pong ORF1 protein comprises at least about 75% or more, at least about 85% or more, at least about 95% or more, or 100% sequence identity with the nucleic acid sequence of SEQ ID NO: 2. In some aspects, the engineered system comprises an expression construct for expressing the Pong ORF1 protein, wherein the expression construct for expressing the Pong ORF1 protein comprises at least about 75% or more, at least about 85% or more, at least about 95% or more, or 100% sequence identity with SEQ ID NO: 100.

In some aspects, the Pong ORF2 protein comprises an amino acid sequence comprising at least about 75% or more, at least about 85% or more, at least about 95% or more, or 100% sequence identity with the amino acid sequence of SEQ ID NO: 3. In some aspects, a nucleic acid sequence encoding the Pong ORF2 protein comprises at least about 75% or more, at least about 85% or more, at least about 95% or more, or 100% sequence identity with the nucleic acid sequence of SEQ ID NO: 4.

The programmable targeting system can be a CRISPR/Cas system comprising a Cas9 nuclease and a guide RNA (gRNA). In some aspects, the Cas9 nuclease comprises an amino acid sequence comprising at least about 75% or more, at least about 85% or more, at least about 95% or more, or 100% sequence identity with the amino acid sequence of SEQ ID NO: 5. In some aspects, the Cas9 nuclease is encoded by a nucleic acid sequence comprising at least about 75% or more, at least about 85% or more, at least about 95% or more, or 100% sequence identity with the nucleic acid sequence of SEQ ID NO: 6. In some aspects, the gRNA comprises a nucleic acid sequence of SEQ ID NO: 65, SEQ ID NO: 66, SEQ ID NO: 67, SEQ ID NO: 80, SEQ ID NO: 113, SEQ ID NO: 67 and SEQ ID NO: 113, or any combination thereof.

When the programmable targeting system is a CRISPR/Cas system comprising a Cas9 nuclease and a guide RNA (gRNA), the transposase can be linked to the Cas9 nuclease. In some aspects, the Pong ORF2 protein is linked to the Cas9 nuclease by one copy of a G4S linker of SEQ ID NO: 64. In some aspects, the Pong ORF2 protein linked to the Cas9 nuclease by one copy of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 106 or a nucleic acid sequence starting at base 8392 to base 14052 of SEQ ID NO: 74. In some aspects, the engineered system comprises an expression construct for expressing the Pong ORF2 protein linked to the Cas9 nuclease by one copy of a G4S linker of SEQ ID NO: 64, wherein the expression construct comprises a nucleic acid sequence comprising at least about 75% or more, at least about 85% or more, at least about 95% or more, or 100% sequence identity with a nucleic acid sequence of SEQ ID NO: 115 or a nucleic acid sequence starting at base 7451 to base 15799 of SEQ ID NO: 74. In some aspects, the cell is an Arabidopsis thaliana cell.

When the programmable targeting system is a CRISPR/Cas system comprising a Cas9 nuclease and a guide RNA (gRNA), the Cas9 nuclease is a dead Cas9 (dCas9) nuclease. In some aspects, the transposase is linked to dCas9. In some aspects, the dCas9 nuclease is linked to Pong ORF2 by one copy of a G4S linker of SEQ ID NO: 64. In some aspects, the Pong ORF2 protein linked to the dCas9 nuclease by one copy of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 110. In some aspects, the engineered system comprises an expression construct for expressing the Pong ORF2 protein linked to the dCas9 nuclease by one copy of a G4S linker of SEQ ID NO: 64, wherein the expression construct comprises a nucleic acid sequence comprising at least about 75% or more, at least about 85% or more, at least about 95% or more, or 100% sequence identity with SEQ ID NO: 115. The genetically modified cell can be an Arabidopsis thaliana cell.

When the programmable targeting system is a CRISPR/Cas system comprising a Cas9 nuclease and a guide RNA (gRNA), the transposase can be linked to the Cas9 nuclease by three copies of a G4S linker of SEQ ID NO: 64. In some aspects, the Pong ORF2 protein linked to the Cas9 nuclease by three copies of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 107. In some aspects, the engineered system comprises an expression construct for expressing the Pong ORF2 protein linked to the Cas9 nuclease by three copies of a G4S linker of SEQ ID NO: 64, wherein the expression construct comprises a nucleic acid sequence comprising at least about 75% or more, at least about 85% or more, at least about 95% or more, or 100% sequence identity with SEQ ID NO: 104. The genetically modified cell can be a soybean cell.

In some aspects, the Pong ORF2 protein is not linked to the targeting nuclease. In some aspects, the engineered system comprises a nucleic acid expression construct for expressing a Cas9 nuclease, wherein the expression construct for expressing the Cas9 nuclease comprises a nucleic acid sequence comprising at least about 75% or more, at least about 85% or more, at least about 95% or more, or 100% sequence identity with SEQ ID NO: 92 or a nucleic acid sequence starting at base 10857 to base 16495 of SEQ ID NO: 94. In some aspects, the engineered system comprises a nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with a nucleic acid sequence of SEQ ID NO 101 or a nucleic acid sequence starting at base 5073 to base 8215 of SEQ ID NO: 89.

The first mPing transposition sequence and the second mPing transposition sequence can flank a cargo polynucleotide. In some aspects, the cargo polynucleotide comprises HSEs. In some aspects, the first mPing transposition sequence comprises at least about 75% or more, at least about 85% or more, at least about 95% or more, or 100% sequence identity with the nucleic acid sequence of SEQ ID NO: 7. In some aspects, the second mPing transposition sequence comprises at least about 75% or more, at least about 85% or more, at least about 95% or more, or 100% sequence identity with the nucleic acid sequence of SEQ ID NO: 8. In some aspects, the donor polynucleotide comprises at least about 75% or more, at least about 85% or more, at least about 95% or more, or 100% sequence identity with the nucleic acid sequence of SEQ ID NO: 81.

In some aspects, the cargo polynucleotide comprises an expression construct for expressing an herbicide resistance function. The herbicide resistance function can be resistance to bialaphos herbicide. In some aspects, the first mPing transposition sequence comprises a nucleic acid sequence comprising at least about 75% or more, at least about 85% or more, at least about 95% or more, or 100% sequence identity with SEQ ID NO: 108. In some aspects, the second mPing transposition sequence comprises a nucleic acid sequence comprising at least about 75% or more, at least about 85% or more, at least about 95% or more, or 100% sequence identity with SEQ ID NO: 109. The cargo polynucleotide can comprise an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97 or SEQ ID NO: 99. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97.

The engineered system can comprise an expression construct for expressing a gRNA for targeting the transposase and nuclease to a target nucleic acid locus in an Arabidopsis thaliana PDS3 gene, wherein the expression construct for expressing the gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74. In some aspects, the engineered system comprises an expression construct for expressing a gRNA for targeting the transposase and nuclease to a target nucleic acid locus in an Arabidopsis thaliana ADH1 gene, wherein the expression construct for expressing a gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89. In other aspects, the engineered system comprises an expression construct for expressing a gRNA for targeting the transposase and nuclease to a target nucleic acid locus in an Arabidopsis thaliana ACT8 gene, wherein the expression construct for expressing a gRNA comprises a nucleic acid sequence comprising at least about 75% or more, at least about 85% or more, at least about 95% or more, or 100% sequence identity with the nucleic acid sequence of SEQ ID NO: 103 or the nucleic acid sequence starting at base 729 to base 1440 of SEQ ID NO: 92. In yet other aspects, the engineered system comprises an expression construct for expressing a gRNA for targeting the transposase and nuclease to a target nucleic acid locus in a soybean DD20 intergenic region, wherein the expression construct for expressing a gRNA comprises a nucleic acid sequence comprising at least about 75% or more, at least about 85% or more, at least about 95% or more, or 100% sequence identity with the nucleic acid sequence of SEQ ID NO: 105.

In some aspects, the engineered system comprises: (a) a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein linked to Cas9 nuclease with one copy of a G4S linker, wherein the expression construct for expressing the Pong ORF2 protein linked to Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74; (c) a donor polynucleotide comprising first and second mPing transposition sequences; and (d) an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence of SEQ ID NO: 103. In some aspects, the donor polynucleotide comprises at least about 75% or more, at least about 85% or more, at least about 95% or more, or 100% sequence identity with the nucleic acid sequence of SEQ ID NO: 81.

In some aspects, the engineered system comprises: (a) a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101; (c) a nucleic acid nucleic acid expression construct for expressing a Cas9 nuclease, wherein the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102; (d) a donor polynucleotide comprising first and second mPing transposition sequences; and (e) an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence of SEQ ID NO: 103. In some aspects, the donor polynucleotide comprises at least about 75% or more, at least about 85% or more, at least about 95% or more, or 100% sequence identity with the nucleic acid sequence of SEQ ID NO: 81.

In other aspects, the engineered system comprises: (a) a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein linked to Cas9 nuclease with three copies of a G4S linker, wherein the expression construct for expressing the Pong ORF2 protein linked to Cas9 nuclease comprises a nucleic acid sequence comprising at least about 75% or more, at least about 85% or more, at least about 95% or more, or 100% sequence identity with the nucleic acid sequence of SEQ ID NO: 104; (c) a donor polynucleotide comprising first and second mPing transposition sequences; and (d) an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence of SEQ ID NO: 105. In some aspects, the donor polynucleotide comprises at least about 75% or more, at least about 85% or more, at least about 95% or more, or 100% sequence identity with the nucleic acid sequence of SEQ ID NO: 99.

In yet other aspects, the engineered system comprises: (a) a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101; (c) a nucleic acid nucleic acid expression construct for expressing a Cas9 nuclease, wherein the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102; (d) a donor polynucleotide comprising first and second mPing transposition sequences; and (e) an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence of SEQ ID NO: 105. In some aspects, the donor polynucleotide comprises at least about 75% or more, at least about 85% or more, at least about 95% or more, or 100% sequence identity with the nucleic acid sequence of SEQ ID NO: 81.

In additional aspects, the engineered system comprises: (a) a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101; (c) a nucleic acid nucleic acid expression construct for expressing a Cas9 nuclease, wherein the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102; (d) a donor polynucleotide comprising first and second mPing transposition sequences; and (e) an expression construct for expressing a gRNA of SEQ ID NO: 67 and a gRNA of SEQ ID NO: 113, wherein the expression construct comprises a nucleic acid sequence comprising at least about 75% or more, at least about 85% or more, at least about 95% or more, or 100% sequence identity with the nucleic acid sequence of SEQ ID NO: 114.

In some aspects, the engineered system comprises: (a) a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein linked to dCas9 nuclease with one copy of a G4S linker, wherein the expression construct for expressing the Pong ORF2 protein linked to Cas9 nuclease comprises a nucleic acid sequence comprising at least about 75% or more, at least about 85% or more, at least about 95% or more, or 100% sequence identity with the nucleic acid sequence of SEQ ID NO: 115; (c) a donor polynucleotide comprising first and second mPing transposition sequences; and (d) an expression construct for expressing a gRNA of SEQ ID NO: 67 and a gRNA of SEQ ID NO: 113, wherein the expression construct comprises a nucleic acid sequence comprising at least about 75% or more, at least about 85% or more, at least about 95% or more, or 100% sequence identity with the nucleic acid sequence of SEQ ID NO: 114.

Another aspect of the instant disclosure encompasses an engineered system for generating a genetically modified cell. The system comprises: (a) a nucleic acid expression construct for expressing a Pong ORF1 protein of a transposase, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein of a transposase linked to a Cas9 nuclease, wherein the expression construct for expressing the Pong ORF2 protein linked to Cas9 nuclease comprises a nucleic acid sequence comprising at least about 75% or more, at least about 85% or more, at least about 95% or more, or 100% sequence identity with the nucleic acid sequence of SEQ ID NO: 104 or the nucleic acid sequence starting at base 7451 to base 14807 of SEQ ID NO: 74; (c) a nucleic acid construct comprising a donor polynucleotide comprising first and second mPing transposition sequences; and (d) an expression construct for expressing a gRNA for targeting the transposase and nuclease to a target nucleic acid locus in the cell. In some aspects, the first mPing transposition sequence comprises a nucleic acid sequence comprising at least about 75% or more, at least about 85% or more, at least about 95% or more, or 100% sequence identity with SEQ ID NO: 7, SEQ ID NO: 108, or SEQ ID NO: 111 and the second mPing transposition sequence comprises a nucleic acid sequence comprising at least about 75% or more, at least about 85% or more, at least about 95% or more, or 100% sequence identity with SEQ ID NO: 8, SEQ ID NO: 109, or SEQ ID NO: 111.

Yet another aspects of the instant disclosure encompasses an engineered system for generating a genetically modified cell The engineered system comprises: (a) a nucleic acid expression construct for expressing a Pong ORF1 protein of a transposase, wherein the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100; (b) a nucleic acid nucleic acid expression construct for expressing a Pong ORF2 protein of a transposase, wherein the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101; (c) a nucleic acid nucleic acid expression construct for expressing a Cas9 nuclease, wherein the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102; (d) a nucleic acid construct comprising a donor polynucleotide comprising first and second mPing miniature inverted-repeat transposable element (MITE) transposition sequences; and (e) an expression construct for expressing a gRNA for targeting the transposase and nuclease to a target nucleic acid locus in the cell. In some aspects, the first mPing transposition sequence comprises a nucleic acid sequence comprising at least about 75% or more, at least about 85% or more, at least about 95% or more, or 100% sequence identity with SEQ ID NO: 7, SEQ ID NO: 108, or SEQ ID NO: 111 and the second mPing transposition sequence comprises a nucleic acid sequence comprising at least about 75% or more, at least about 85% or more, at least about 95% or more, or 100% sequence identity with SEQ ID NO: 8, SEQ ID NO: 109, or SEQ ID NO: 111.

One aspect of the instant disclosure encompasses one or more nucleic acid constructs for generating a genetically modified cell. The one or more constructs encode an engineered nucleic acid modification system. The nucleic acid modification system can be as described above.

Another aspect of the instant disclosure encompasses a cell comprising an engineered nucleic acid modification system for generating a genetically modified cell or one or more nucleic acid constructs for generating a genetically modified cell. The engineered nucleic acid modification system and the one or more nucleic acid constructs can be as described herein above. In some aspects, the cell is a eukaryotic cell. In some aspects, the eukaryotic cell is a plant cell, a plant or part thereof, or seed.

An additional aspect of the instant disclosure encompasses a method of targeted insertion of a nucleic acid sequence into a target nucleic acid locus in a cell. The method comprises introducing one or more nucleic acid constructs for generating a genetically modified cell encoding an engineered nucleic acid modification system into the cell. The method also comprises maintaining the cell under conditions and for a time sufficient for the donor polynucleotide to be inserted in the target locus; and optionally identifying an insertion of the donor polynucleotide in the nucleic acid locus in the cell. The engineered nucleic acid modification system and the one or more nucleic acid constructs can be as described herein above. In some aspects, the cell is a eukaryotic cell. In some aspects, the eukaryotic cell is a plant cell, a plant or part thereof, or seed. In some aspects, the cell is ex vivo.

One aspect of the instant disclosure encompasses a kit for generating a genetically modified cell. The kit comprises a nucleic acid modification system for generating a genetically modified cell or one or more nucleic acid constructs for generating a genetically modified cell. Each of the engineered systems generates an engineered cell comprising an accurate insertion of the donor polynucleotide into the target nucleic acid locus. The engineered nucleic acid modification system and the one or more nucleic acid constructs can be as described herein above. In some aspects, the kit comprises one or more cells comprising one or more engineered systems, one or more nucleic acid constructs, or combinations thereof. In other aspects, the one or more cells are eukaryotic. In some aspects, the one or more eukaryotic cells comprise a plant cell, a plant or part thereof, or seed.

### BRIEF DESCRIPTION OF THE FIGURES

The patent or application file contains at least one drawing executed in color. Copies of this patent or patent application publication with color drawing(s) will be provided by the Office upon request and payment of the necessary fee

FIG. 1 is a diagram depicting an engineered system excising a donor polynucleotide from a donor site in a plant and inserting the excised donor polynucleotide into a locus in the Arabidopsis PDS3 gene.

FIG. 2 depicts a schematic overview of twelve different transgenes comprising Cas9 and derivative proteins linked either to the N- or C-terminus of Pong transposase ORF1 (blue) or to the N- or C-terminus of Pong ORF2 (orange) protein coding regions. Three different versions of Cas9 were used: double-strand cleavage Cas9, the single stranded nickase deCas9, and the catalytically dead dCas9.

FIG. 3A. The functional verification of ORF1/2 and Cas9 fusion proteins. GFP fluorescence was detected for all 12 fusion proteins as well as the ORF1/ORF2 positive control, since mPing excision from the GFP donor site restores the GFP expression. The negative control without ORF1/ORF2 (−ORF1-ORF2) was not able to excise mPing.

FIG. 3B. The functional verification of ORF1/2 and Cas9 fusion proteins. A functional CRISPR/Cas9 system when linked to ORF1/2 was verified through the observation of white seedlings and sectors in plants generated from the Cas9 targeting of the Arabidopsis PDS3 gene with all four Cas9 fusion proteins. Three examples of individual plants are shown.

FIG. 4A. Screening insertions. PCR strategy to detect targeted insertions into the PDS3 gene. mPing can insert in the forward or reverse orientation relative to PDS3.

FIG. 4B. Screening insertions. PCR with negative controls: a line lacking the ORF1/ORF2 proteins (mPing only), lacking Cas9 (mPing+ ORF1/ORF2) and a no template PCR (−). The expected amplification sizes are indicated by black arrowheads. The correct PCR products validated by Sanger sequencing are marked with red arrows.

FIG. 4C. Screening insertions. Replicate of the PCR from clone #2 in FIG. 4B. This PCR displays the correct sized and sequenced bands (red arrows) in each reaction.

FIG. 5 depicts nucleic acid sequences at insertion sites of 9 unique transposition events. The sequence of the mPing transposable element is green. The target site duplication sequence is red. The guide RNA target site is grey highlighted. The PDS gene is unhighlighted black. For simplicity, only the mPing/PDS3 junction of these sequences are shown.

FIG. 6A. PCR strategy to determine if any transgenic DNA would insert at a Cas9 cleavage site. The PCR shows no bands of expected size (black arrowheads), which demonstrates that mPing insertion from FIG. 4 is a product of transposition, and not random.

FIG. 6B. Testing if the single components of the system could recapitulate the results. No Cas9 and ORF1/2 (mPing only), no Cas9 (+ORF1/2), and no ORF1/2 (+Cas9) controls each failed to produce the expected band and therefore cannot generate targeted insertions. Having Cas9 and ORF1/2, but in an un-linked configuration, produced targeted insertion. The lane to the far right is clone #2 from FIG. 4, which is used as a positive control in this experiment. The four gels represent the same four PCR assays from FIG. 4A. Black arrowheads denote the expected size of the targeted insertion in each PCR.

FIG. 7A is a diagram showing the three systems designed with gRNAs targeted to three different target loci: the PDS3 gene, the ADH1 gene, and the promoter of ACT8 gene.

FIG. 7B are the Sanger sequencing results of junctions of target insertions into the PDS3 gene, the ADH1 gene, and the promoter of ACT8 gene. The sequence below mPing is the expected sequence of a perfect “seamless” insertion. The chromatograms above the sequence show the sequences at the insertion sites. The highlighted bases are 1-2 nucleotide insertions or deletions.

FIG. 8A depicts a PCR strategy to detect targeted insertions into the PDS3 gene. mPing can insert in either the forward direction (above the PDS3 region) or reverse direction (below the PSD3 region). The location of 4 PCR primers (R,L,U,D) are shown for orientation.

FIG. 8B depicts an agarose gel run of PCR products using primers from FIG. 8A from systems comprising ORF1 and 2 linked or unlinked to Cas9 nuclease. Arrowheads denote the correct size of the PCR products for each set of primers. No Cas9 and ORF1/2 (“mPing only”), no Cas9 (“+ORF1/2”), and no ORF1/2 (“+Cas9”) are negative controls and showed no bands.

FIG. 9A is a diagram of a vector that contains the CRISPR/Cas9 system (including gRNA), the mPing donor element, and ORF1 and ORF2 transposase proteins.

FIG. 9B depicts a PCR strategy to detect targeted insertions into the PDS3 gene using the vector of FIG. 9A. mPing can insert in either the forward direction (above the PDS3 region) or reverse direction (below the PSD3 region). The location of 4 PCR primers (R,L,U,D) are shown for orientation.

FIG. 9C depicts PCR detection of mPing targeted insertion in the Arabidopsis genome using the vector in FIG. 9A. PCR detection used primer sets from FIG. 9B.

FIG. 10 depicts targeted insertion based on the Pong/mPing transposon system. Fusion of the Pong transposase ORFs with Cas9 provides the transposase sequence specificity for the insertion of the non-autonomous mPing element. The mPing element is excised out of a donor site provided on the transgene, generating fluorescence. mPing insertion at the target site is screened for by PCR.

FIG. 11 depicts the Experimental Design of Protein Fusions and Testing. Twelve different transgenes where created and transformed into Arabidopsis. Cas9 and derivative proteins where linked either to the Pong transposase ORF1 (blue) or ORF2 (orange) protein coding regions. Both N- and C-terminal fusions were created. Three different versions of Cas9 were used: double-strand cleavage Cas9, the single stranded nickase deCas9, and the catalytically dead dCas9. When a functional transposase protein is generated by expression of ORF1 and ORF2, it excises the mPing transposable element out of the 35S-GFP donor location, producing fluorescence. The goal of this project was to demonstrate user-defined targeted insertion of the mPing transposable element by programming the CRISPR-Cas9 system with a custom guide RNA.

FIG. 12A depicts photographs showing fluorescence generated upon excision of mPing from the 35S: GFP donor site. mPing only transposes in the presence of both ORF1 and ORF2 transposase proteins, and fusing ORF2 to Cas9 still results in mPing excision.

FIG. 12B depicts a PCR gel showing excision as in FIG. 12A assayed by PCR using primers at the 35S: GFP donor site. A smaller sized band is generated upon mPing excision.

FIG. 12C depicts a PCR assay to detect targeted insertion of mPing at PDS3 gene. Primer names (U,L,R,D) and locations are listed above. Targeted insertion is detected via PCR in plants that have all three proteins: ORF1, ORF2 and Cas9. Targeted insertions are detected when ORF2 and Cas9 are physically linked, or when unlinked but present in the same cells.

FIG. 12D depicts a cartoon of mPing excision and targeted insertion when ORF2 is linked to Cas9.

FIG. 12E depicts an example of a Sanger sequence read of the junction between the PDS3 gene and the targeted insertion of mPing.

FIG. 12F depict sequence analysis of 17 distinct insertion events of mPing at PDS3. mPing sequences are shown in yellow, and the target site duplication of TTA/TAA from the donor site is shown in red. Within the PDS3 target site, the gRNA targeted sequence is shown in grey. The mPing is inserted between the third and fourth base of the gRNA target sequence (black arrowhead). The variation of the sequence found on either end of the insertion site is shown.

FIG. 12G depicts a plot showing the number of SNPs at the insertion site identified by Sanger sequencing targeted insertion events.

FIG. 13A depicts photographs showing the functional verification of ORF1/2 and Cas9 fusion proteins. GFP fluorescence was detected for all 12 fusion proteins as well as the ORF1/ORF2 positive control, since mPing excision from the GFP donor site restores the GFP expression. The negative control without ORF1/ORF2 (−ORF1-ORF2) was not able to excise mPing.

FIG. 13B depict the functional verification of ORF1/2 and Cas9 fusion proteins. A functional CRISPR/Cas9 system when linked to ORF1/2 was verified through the observation of white seedlings and sectors in plants with all four Cas9 fusion proteins. Three examples of individual plants are shown.

FIG. 14A depicts a PCR strategy to detect targeted insertions into the PDS3 gene. mPing can insert in the forward or reverse orientation relative to PDS3.

FIG. 14B depicts an electrophoresis gel of PCR products with negative controls: a line lacking the ORF1/ORF2 proteins (mPing only), lacking Cas9 (mPing+ORF1/ORF2) and a no template PCR (−). The expected amplification sizes are indicated by black arrowheads. The correct PCR products are marked with red arrows.

FIG. 14C depicts screening insertions. Replicate of the PCR from clone #2. This PCR displays the correct sized bands (red arrows) in each reaction.

FIG. 15 depicts the comparison of the number of base deletions (left of zero on the X-axis) and insertions (right of zero on the X-axis) for two configurations of Cas9 and ORF2: linked and unlinked. Insertions of mPing (red) into PDS3 (blue) were subject to amplicon deep sequencing and each junction analyzed separately. Since mPing can insert in either orientation (black arrows within red mPing elements), four distinct junction points are analyzed. The size of the black filled circle represents the percentage of deep sequenced reads.

FIG. 16A depict additional controls. PCR strategy to determine if any transgenic DNA would insert at a Cas9 cleavage site. The PCR shows no bands, which demonstrates that mPing insertion from FIGS. 12A-13B is a product of transposition, and not random.

FIG. 16B depict additional controls. Testing if the single components of our system could recapitulate our results. No Cas9 and ORF1/2 (mPing only), no Cas9 (+ORF1/2), and no ORF1/2 (+Cas9) controls each failed to produce the expected band and therefore cannot generate targeted insertions. Having Cas9 and ORF1/2, but in an un-linked configuration, produced targeted insertion. The lane to the far right is clone #2 from FIGS. 12-12G, which is used as a positive control in this experiment. The four gels represent the same four PCR assays from FIG. 12A. Black arrowheads denote the expected size of the targeted insertion in each PCR.

FIG. 17A depicts an overview of targeted insertion at 3 distinct loci. By switching the CRISPR gRNA, distinct regions of the genome are targeted for mPing insertion.

FIG. 17B depicts how mPing can insert into DNA for both directions. Arrows indicate primers used to detect target insertions: U, upstream of target gene; D, downstream of target gene; R, right end of mPing; L, left end of mPing. PCR products were then purified and sequenced.

FIG. 17C depicts sanger sequencing chromatograms for junctions of target insertions into an additional target besides PDS3: ADH1.

FIG. 17D depicts sanger sequencing chromatograms for junctions of target insertions into an additional target besides PDS3: ACT8 promoter.

FIG. 18 depicts analysis of the left and right junctions of mPing targeted insertions upstream of the ACT8 gene in T2 plants with Cas9 linked to ORF2. Single individual T2 plants were assayed one-by-one, and 8 plants were confirmed by Sanger sequencing to have targeted insertions of mPing.

FIG. 19A. Addition of 6 heat shock element (HSE) sequences originally upstream of a heat-shock responsive gene into mPing and cartoon of attempted targeted insertion upstream of the ACT8 gene. The individual HSEs are shown as red bars in the mPing-HSE element.

FIG. 19B. PCR gel of mPing element excision from the donor location demonstrating that the modified mPing-HSE element could excise properly. The Sspl digest is performed to improve the assay's sensitivity. AtADH1 is shown as a PCR control.

FIG. 19C PCR gel detecting targeted insertions. Both a pool of T2 plants was assayed, as well as four individual T2 generation plants. Bands with red arrow heads are the correct size and were Sanger sequenced to demonstrate the correct targeted insertion into the promoter region of the ACT8 gene. AtADH1 is shown as a PCR control.

FIG. 19D Sanger sequencing results of the junction of mPing-HSE inserted at its target site upstream of the ACT8 gene. The red highlighted two bases are deleted compared to the predicted seamless insertion.

FIG. 19E Sanger sequencing through the mPing-HSE element inserted upstream of ACT8 as in FIG. 19D. The PCR primers used to generate this amplicon are whose above. Below, all 6 delivered HSEs are shown as red arrows and in this example a 11 base deletion is detected at the junction between mPing-HSE and the upstream region of ACT8.

FIG. 20 depicts experimental design to use targeted transposition of a modified mPing element in order to transcriptionally rewire the ACT8 gene. The goal is to engineer the ACT8 gene have transcriptional activation during heat stress.

FIG. 21A depicts a map of the vector testing the ability of unlinked Cas9 Nickase to direct targeted insertions of mPing. Targeted insertion into ADH1 has been detected at a low frequency and sequenced. This insertion shows the left junction of mPing at ADH1 with a 14 bp deletion.

FIG. 21B depicts further experimentation demonstrating that dCas9 can participate in targeted insertion when two gRNAs are used. In this case, the transposase is inserting mPing at a TTA site nearby the gRNA target sites. The Sanger sequencing of one end of mPing is shown.

FIG. 21C depicts the experimental design to use of two gRNAs and a catalytically active Cas9 protein. In this example, a region of DNA is cut out of the genome with two gRNAs and replaced with mPing.

FIG. 21D PCR primer placement for screening mPing targeted insertion.

FIG. 21E shows targeted insertion screening assay. Red arrowheads are PCR products that were Sanger sequenced and verified targeted insertions.

FIG. 21F shows one end of a targeted insertion that replaces the DNA inbetween the two gRNAs used.

FIG. 22A Vector maps of TDNAs used for a two-step (two-component) transformation. The donor vector was transformed into Arabidopsis first, and a stable transgenic line was used for a second transformation using the helper vector.

FIG. 22B The one-component vector containing both donor TE (mPing) and helpers (ORF1, ORF2-Cas9) was also tested to be able to direct targeted insertion. Blue triangles are LB and RB ends of the T-DNA. Arrows denote promoters, and black boxes are terminators. The mPing donor TE is shown in red.

FIG. 23A depicts the vector for transposase-mediated targeted insertion of mPing into the soybean (Glycine max) crop genome. Soybean transformation vector with a gRNA that targets the “DD20” non-protein coding region of the soybean genome, using an unlinked ORF2 and Cas9 configuration.

FIG. 23B depicts the vector for transposase-mediated targeted insertion of mPing into the soybean (Glycine max) crop genome. Similar vector as in FIG. 23A, but with a linked ORF2 and Cas9.

FIG. 23C depicts the transposase-mediated targeted insertion of mPing into the soybean (Glycine max) crop genome. The overall goal of targeted insertion of mPing into the DD20 non-protein coding region of the soybean genome without previously integrating and new sequences such as a landing pad for targeted insertion.

FIG. 23D depicts the transposase-mediated targeted insertion of mPing into the soybean (Glycine max) crop genome. PCR primer strategy to detect targeted insertion (top) and PCR gel (bottom). Bands with red arrowheads are the correct size and were validated by Sanger sequencing. Two out of nine transgenic soybean plants showed targeted insertion of mPing.

FIG. 23E depicts the transposase-mediated targeted insertion of mPing into the soybean (Glycine max) crop genome. Top is the Sanger sequence example of a targeted insertion into the soybean genome (plant R0 #8 from FIG. 23D). Bottom is an example of mPing-HSE inserted into DD20 in the soybean genome.

FIG. 23F depicts the constructs used for transposase-mediated targeted insertion of mPing into the soybean (Glycine max) crop genome. The seven mPing constructs test how to functionally fuse ORF2 to Cas9 in soybean, and if the mPing-HSE and mPing-bar cargos can be delivered to specific sites in the soybean genome.

FIG. 23G depicts the transposase-mediated targeted insertion of mPing into the soybean (Glycine max) crop genome. The percent of plants tested with excision of mPing (top left), mutagenesis of the target location by Cas9 (top right), plants with combined excision and mutagenesis (bottom left), and targeted insertion of mPing at the DD20 location in the soybean genome (bottom right).

FIG. 24A depicts the four mPing constructs used to determine mPing sequences required for transposition and to test longer cargo sequences. Each of these has the tested capability to excise from the genome and participate in targeted integration.

FIG. 24B depicts an electrophoresis gel of PCR products testing the ability of the mPing constructs from FIG. 24A to excise out of the donor position. Blue triangle denote the size of the mPing constructs at the donor site, and the smaller band the same position after successful mPing excision. The mPing element with only the TIRs (mPing TIR_bar gene) does not excise efficiently.

FIG. 24C depicts an electrophoresis gel of PCR products targeted insertion of mPing and the mPing_bar CDS to the non-coding region upstream of the ACTIN8 gene. Red triangles denote the correct PCR product for a targeted insertion.

FIG. 25A depicts an electrophoresis gel of PCR products showing the excision of each of the mPing derived constructs mPing_bar CDS and mPing_bar gene from the donor position. Each pool of plants displays mPing excision.

FIG. 25B depicts the PCR strategy and primer placement for screening targeted insertion events. The mPing-bar CDS and mPing-bar versions of mPing can insert into the targeted location in either orientation.

FIG. 25C depicts an electrophoresis gel of PCR products showing the targeted insertion of mPing_bar CDS and mPing_bar gene upstream of the ACTIN8 gene. Red triangles denote PCR products of the correct size for a targeted insertion event.

FIG. 25D depicts the rate of mPing element excision (left) and targeted insertion (right) for different mPing versions in T1 Arabidopsis plants.

FIG. 26A depicts a map of the construct comprising the bar CDS in mPing inserted into the ACT8 gene. This insertion shows the right junction of mPing_bar CDS at ACT8 with a 2 bp deletion.

FIG. 26B shows Sanger sequencing results of bar CDS in mPing inserted into the ACT8 gene of FIG. 26A aligned to the expected sequence of targeted insertion showing the 2 bp deletion. Red regions are mPing sequence, grey highlighted are the bar gene coding region, and green is the promoter region upstream of ACT8.

FIG. 27A depicts a map of the construct comprising the bar gene with the bar promoter and terminator elements in mPing inserted into the ACT8 gene. This insertion shows the right junction of mPing_bar gene at ACT8 with a 2 bp deletion.

FIG. 27B shows Sanger sequencing results of bar in mPing inserted into the ACT8 gene of FIG. 27A aligned to the expected sequence of targeted insertion showing the 2 bp deletion. Red regions are mPing sequence, grey highlighted are the Nos promoter+bar gene+Nos terminator, and green is the promoter region upstream of ACT8.

FIG. 28A shows that the mPing-bar targeted insertion confers the herbicide resistance trait. Amplicons “PCR1” to “PCR6” are used to genotype for the presence of the mPing-bar transgene in R0 transformed soybean plants.

FIG. 28B shows PCR results of the PCR targets in FIG. 28A. GmLe1 is a control gene.

FIG. 28C shows PCR primer placement in order to assay for the mPing-bar targeted insertion.

FIG. 28D shows the PCR assay for targeted insertion in the DD20 targeted location in the soybean genome. Red arrowheads denotes targeted insertions that were verified by Sanger sequencing.

FIG. 29A is a diagrammatic depiction of sequential transformation of DD45::Cas9 plants with mPing construct containing all components of the system, except Cas9.

FIG. 29B is the excision assay of mPing out of the donor transgene.

FIG. 29C is the PCR to detect targeted insertions.

FIG. 29D is the Sanger sequencing of a targeted insertion of mPing into the ACT8 region of the Arabidopsis genome.

FIG. 29E is a diagram of the measurement of the rate of excision and targeted insertion in the DD45::Cas9 line.

### DETAILED DESCRIPTION

The present disclosure encompasses engineered nucleic acid modification systems and methods of using the engineered systems for generating genetically modified cells and organisms. Unlike currently available insertion systems that rely on homologous recombination or homology-directed repair for inserting or replacing a nucleic acid sequence, the engineered systems and methods of the disclosure can efficiently mediate controlled and targeted insertion of a polynucleotide of choice to generate a genetically modified cell having an insertion of the polynucleotide at a target nucleic acid locus in a gene of interest. In some aspects, the insertion replaces a nucleic acid sequence in the cell. Importantly, the disclosed engineered systems and methods can efficiently mediate targeted insertion of polynucleotides even in organisms where such genetic manipulation is known to be problematic, including plants. Further, the compositions and methods can insert polynucleotides without introducing unwanted mutations in the transferred polynucleotide or in the nucleic acid sequences at the target nucleic acid locus. The engineered system can accomplish that by combining the targeting capabilities of a targeting nuclease, with the insertion capability and ability to seamlessly resolve the junction without mutation of a transposase. This is important because this mechanism bypasses the host-encoded homologous recombination step or damage repair pathways normally used when a polynucleotide is introduced. Surprisingly and unexpectedly, the engineered systems can simultaneously target more than one locus.

### I. Composition

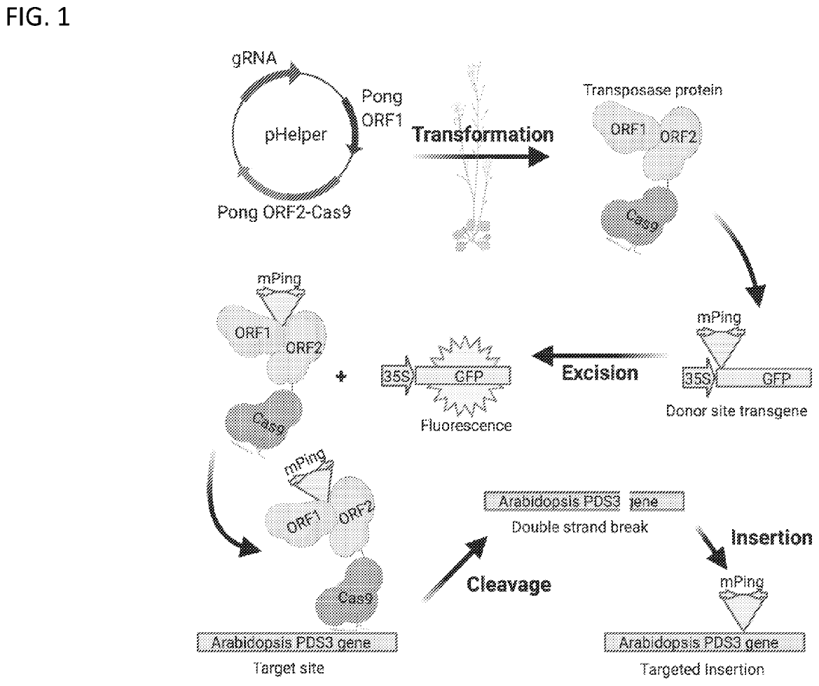

One aspect of the present disclosure encompasses an engineered nucleic acid modification system (the “engineered system”) for generating a genetically modified cell. The engineered system comprises a transposase, a donor polynucleotide, and a programmable targeting system that can be programmed to target the transposase and the donor polynucleotide to a target nucleic acid locus in the cell, thereby accomplishing insertion of the donor polynucleotide at the target nucleic acid locus to generate a genetically modified cell comprising the donor polynucleotide inserted at the target nucleic acid locus (FIG. 1). The programmable targeting system, the transposase, and the donor polynucleotide are described in further detail below.

### (a) Transposase

The engineered system of the instant disclosure comprises a transposase. As used herein, the term “transposase” refers to a protein or a protein fragment derived from any transposable element (TE), wherein the transposase is capable of cutting or copying a donor polynucleotide from a nucleic acid sequence comprising the donor polynucleotide, protecting the donor polynucleotide from degradation by binding to transposable element sequences in the donor polynucleotide, inserting the donor polynucleotide at a target locus, or any combination thereof. TEs can be assigned to any one of two classes according to their mechanism of transposition, which can be described as either copy and paste (Class I TEs) or cut and paste (Class II TEs).

Class I TEs are retrotransposons that copy and paste themselves into different genomic locations in two stages: first, TE nucleic acid sequences are transcribed from DNA to RNA, and the RNA produced is then reverse transcribed to DNA. This copied DNA is then inserted back into the genome at a new position. The reverse transcription step is catalyzed by a reverse transcriptase activity, which is often encoded by the TE itself. Non-limiting examples of Class I TEs include Tnt1, Opie, Huck, and BARE1.

The transposition mechanism of Class II TEs does not involve an RNA intermediate. The transpositions are catalyzed by a transposase enzyme that cuts the target site, cuts out the transposon or copies the transposon, and positions it for ligation into the target site. Non-limiting examples of Class II TEs include P Instability Factor (PIF), Pong, Ac/Ds, Pong TE or Pong-like TEs, Spm/dSpm, Harbinger, P-elements, Tn5 and Mutator.

Transposases generally recognize and interact with compatible transposition sequences at the ends of the TE to mediate transposition of the TE. For instance, the transposase can bind the transposition sequences at the terminal ends of the TE and can cleave the DNA, removing the TE from the excision/donor site, can protect the TE ends from degradation while it is outside the chromosome, and can cleave the insertion site at a new location in the genome of a cell and integration of the TE at the insertion site. One or more of these functions of the transposase can be used in an engineered system of the instant disclosure for effective insertion of a donor polynucleotide. For Class I TEs, the transposases of some TEs recognize the terminal transposition sequences at the ends of an RNA transcript of the TE, reverse transcribe the transcript into DNA, then cleave and integrate the TE at the insertion site. Accordingly, a transposase of the instant disclosure can be any transposase or fragment thereof, provided the transposase recognizes the compatible terminal transposition sequences of the donor polynucleotide and mediates insertion of the polynucleotide at the target locus. Transposition sequences compatible with the transposase can be as described in Section I(b) below.

In an engineered system of the instant disclosure, a transposase recognizes the transposition sequences of the donor polynucleotide. When the transposase is derived from a Class I TE, the transposase first transcribes the donor polynucleotide into an RNA transcript and reverse transcribes the RNA transcript to DNA for insertion at the target locus. When the transposase is derived from a Class II TE, the transposase first cleaves or copies the donor polynucleotide from a source nucleic acid sequence such as a nucleic acid construct encoding the donor polynucleotide for insertion at the target locus. The transposase remains bound to the polynucleotide, protecting this molecule from degradation while it is outside the chromosome. In some aspects, the transposase also cleaves the target locus before inserting the donor polynucleotide. In other aspects, the nucleic acid sequence at the target is cleaved by a nuclease function of a programmable targeting system of the instant disclosure as described in Section I(c) herein below.

In some aspects, the transposase is derived from a Class II TE. In some aspects, the transposase is derived from the P Instability Factor (PIF) TE or PIF-like TEs. In some aspects, a transposase of the instant disclosure is a split transposase. In some aspects, the transposase is a Pong or Pong-like transposase comprising a Pong ORF1 protein and a Pong ORF2 protein. The transposases of the Pong and Pong-like TEs are split transposases comprising a first protein encoded by open reading frame 1 (ORF1 protein) and a second protein encoded by open reading frame 2 (ORF2 protein) of the TE.

Accordingly, when a transposase of the instant disclosure is a Pong or Pong-like transposase, the engineered system comprises both ORF1 and ORF2 proteins. In some aspects, the Pong ORF1 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. In some aspects, the Pong ORF1 protein comprises an amino acid sequence comprising at least about 75% or more, at least about 85% or more, at least about 95% or more, or 100% sequence identity with the nucleic acid sequence of SEQ ID NO: 1. In some aspects, a nucleic acid sequence encoding the Pong ORF1 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 2. In some aspects, a nucleic acid sequence encoding the Pong ORF1 protein comprises at least about 75% or more, at least about 85% or more, at least about 95% or more, or 100% sequence identity with the nucleic acid sequence of SEQ ID NO: 2.

In some aspects, the Pong ORF2 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino sequence of SEQ ID NO: 3. In some aspects, the Pong ORF2 protein comprises an amino acid sequence comprising at least about 75% or more, at least about 85% or more, at least about 95% or more, or 100% sequence identity with the amino acid sequence of SEQ ID NO: 3. In some aspects, a nucleic acid sequence encoding the Pong ORF2 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 4. In some aspects, a nucleic acid sequence encoding the Pong ORF2 protein comprises at least about 75% or more, at least about 85% or more, at least about 95% or more, or 100% sequence identity with the nucleic acid sequence of SEQ ID NO: 4.

### (b) Donor Polynucleotide

Engineered systems of the disclosure also comprise a donor polynucleotide. In the presence of the transposase and the programmable targeting nuclease of the engineered system of the instant disclosure, the donor polynucleotide is cut or copied from a nucleic acid sequence comprising the donor polynucleotide and targeted by the programmable targeting system to a target nucleic acid locus to thereby mediate insertion of the donor polynucleotide into the target nucleic acid locus. A donor polynucleotide comprises a first transposition sequence at a first end of the donor polynucleotide, and a second transposition sequence at a second end of the donor polynucleotide. The transposition sequences are compatible with the transposase of a engineered system of the instant disclosure. As used herein, the term “compatible” when referring to transposition sequences refers to transposition sequences that can be recognized by a transposase of the instant disclosure for transposition of the donor polynucleotide in the cell.

In some aspects, the transposition sequences are derived from the TE from which the transposase is derived. However, the transposition sequences can also be derived from TEs other than the TE from which the transposases are derived, provided the transposition sequences are compatible with the transposon of the engineered system. Transposition sequences of the instant disclosure can be derived from autonomous or non-autonomous TEs. Non-autonomous TEs have short internal sequences devoid of open reading frames (ORF) that encode a defective transposase, or do not encode any transposase. Non-autonomous elements transpose through transposases encoded by autonomous TEs. The transposition sequences of the donor polynucleotide can each have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with transposition sequences of the TE from which they are derived.

As explained in Section I(a) above, the transposase recognizes the transposition sequences and mediates the insertion of the donor polynucleotide into the desired target locus. A donor polynucleotide can be an RNA polynucleotide or a DNA polynucleotide. The transposition sequence can flank cargo nucleic acid sequences of interest, and insertion of the donor polynucleotide can result in the insertion of the cargo nucleic acid sequences of interest into the desired target locus. Non-limiting examples of cargo nucleic acid sequences that can be of interest for inserting in a target locus can be as described in Section IV herein below.

Further, insertion of the donor polynucleotide in a target locus can alter the function of the target locus. For instance, insertion of a donor polynucleotide in a nucleic acid sequence encoding a reporter can inactivate the reporter, thereby indicating a successful integration event. Conversely, excision of a donor polynucleotide from a nucleic acid sequence encoding a reporter can re-activate the reporter, thereby indicating a successful excision event.

In some aspects, the engineered system further comprises a reporter nucleic acid construct for expressing a reporter, wherein the reporter nucleic acid construct comprises a promoter operably linked to a polynucleotide sequence encoding the reporter, wherein the donor polynucleotide is inserted in the reporter nucleic acid construct thereby inactivating expression of the reporter, and wherein expression of the reporter is activated by excision of the inserted donor polynucleotide from the reporter nucleic acid construct by the transposase. The reporter can be a GFP reporter.

In some aspects, the transposase of the instant disclosure is derived from a PIF or PIF-like TE, and the transposition sequences compatible with the transposase are derived from a PIF or a PIF-like TE from which the transposase is derived, or can be derived from a tourist-like miniature inverted-repeat transposable element (MITE). In some aspects, the transposase is derived from a Pong, a Pong-like, Ping, or a Ping-like TE, and the transposition sequences compatible with the transposase can be derived from a stowaway-like MITE. In some aspects, the transposase is derived from a Pong, a Pong-like, a Ping, or a Ping-like TE, and the transposition sequences compatible with the transposase are derived from an mPing or mPing-like MITE.

In some aspects, the transposition sequences are a first and second transposition sequences of a miniature inverted-repeat transposable element (MITE). In some aspects, the MITE is an mPing MITE. In some aspects, mPing comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 96. In some aspects, mPing comprises a nucleotide sequence comprising at least about 75% or more, at least about 85% or more, at least about 95% or more, or 100% sequence identity with the nucleic acid sequence of SEQ ID NO: 96.

Importantly, it is noted that the inventors discovered that including mPing MITE first and second transposition sequences longer than the inverted repeats which was recognized by the art as being sufficient for transposition, significantly enhanced efficiency of transposition in a engineered system of the instant disclosure. Accordingly, transposition sequences of the instant disclosure can comprise the mPing inverted repeat 1 and inverted repeat 2 and further comprise mPing sequences flanked (internal to) by the mPing inverted repeat 1 and inverted repeat 2. For instance, transposition sequences of the mPing MITE can comprise the mPing inverted repeat 1, and further comprise any number of nucleotides of mPing downstream of inverted repeat 1 and any number of nucleotides of mPing downstream of inverted repeat 2.

In some aspects, transposition sequences of the mPing MITE comprise mPing inverted repeat 1 and inverted repeat 2. In some aspects, mPing inverted repeat 1 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. In some aspects, mPing inverted repeat 1 comprises a nucleotide sequence comprising at least about 75% or more, at least about 85% or more, at least about 95% or more, or 100% sequence identity with the nucleic acid sequence of SEQ ID NO: 7.

In some aspects, mPing inverted repeat 2 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 8. In some aspects, mPing inverted repeat 2 comprises a nucleotide sequence comprising at least about 75% or more, at least about 85% or more, at least about 95% or more, or 100% sequence identity with the nucleic acid sequence of SEQ ID NO: 8.

In some aspects, transposition sequences of the mPing MITE comprise the mPing inverted repeat 1 and inverted repeat 2 and further comprise mPing sequences flanked (internal to) by the mPing inverted repeat 1 and inverted repeat 2. In some aspects, transposition sequences of the instant disclosure comprise a first mPing transposition sequence comprising a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 111. In some aspects, transposition sequences of the instant disclosure comprise a first mPing transposition sequence comprising a nucleotide sequence comprising about 75% or more, at least about 85% or more, at least about 95% or more, or 100% sequence identity with the nucleic acid sequence of SEQ ID NO: 111.

In some aspects, transposition sequences of the instant disclosure comprise a second mPing transposition sequence comprising a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 112. In some aspects, transposition sequences of the instant disclosure comprise a second mPing transposition sequence comprising a nucleotide sequence comprising about 75% or more, at least about 85% or more, at least about 95% or more, or 100% sequence identity with the nucleic acid sequence of SEQ ID NO: 112.

In some aspects, transposition sequences of the instant disclosure comprise a first mPing transposition sequence comprising a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 108. In some aspects, transposition sequences of the instant disclosure comprise a first mPing transposition sequence comprising a nucleotide sequence comprising about 75% or more, at least about 85% or more, at least about 95% or more, or 100% sequence identity with the nucleic acid sequence of SEQ ID NO: 108.

In some aspects, transposition sequences of the instant disclosure comprise a second mPing transposition sequence comprising a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 109. In some aspects, transposition sequences of the instant disclosure comprise a second mPing transposition sequence comprising a nucleotide sequence comprising about 75% or more, at least about 85% or more, at least about 95% or more, or 100% sequence identity with the nucleic acid sequence of SEQ ID NO: 109.

In some aspects, the donor polynucleotide comprises a nucleotide sequence comprising heat shock element (HSE) sequences flanked by mPing first and second transposition sequences. In some aspects, the donor polynucleotide comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 512 of SEQ ID NO: 81 or the nucleic acid sequence starting at base 69 to base 512 of SEQ ID NO: 93. In some aspects, the donor polynucleotide comprises a nucleic acid sequence comprising at least about 75% or more, at least about 85% or more, at least about 95% or more, or 100% sequence identity with the nucleic acid sequence starting at base 69 to base 512 of SEQ ID NO: 81 or the nucleic acid sequence starting at base 69 to base 512 of SEQ ID NO: 93.

In some aspects, the nucleic acid construct comprising the donor polynucleotide comprises an expression construct for expressing a herbicide resistance function. In some aspects, the herbicide resistance function is resistance to bialaphos herbicide. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 97 or SEQ ID NO: 99. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97 or SEQ ID NO: 99. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 97. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97.

The engineered system can further comprise a nucleic acid expression construct comprising a promoter operably linked to a polynucleotide sequence encoding a GFP reporter, wherein the donor polynucleotide is inserted in the nucleic acid expression construct. In some aspects, the nucleic acid expression construct comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the nucleic acid expression construct comprises at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

### (c) Programmable Targeting System

The engineered system comprises a programmable targeting system. A programmable targeting system can be any single or group of components capable of targeting components of the engineered system to a target nucleic acid locus, to introduce a cut in the target nucleic acid locus, or both to thereby accomplish insertion of the donor polynucleotide into the target locus. The target nucleic acid locus can be in a coding or regulatory region of interest or can be in any other location in a nucleic acid sequence of interest. A gene can be a protein-coding gene, an RNA coding gene, or an intergenic region. The target nucleic acid locus can be in a nuclear, organellar, or extrachromosomal nucleic acid sequence. The cell can be a eukaryotic cell. In some aspects, the cell is a plant cell. In some aspects, the plant is a soybean plant.

A programmable targeting system generally comprises a programmable, sequence-specific nucleic acid-binding domain. In some aspects, the programmable targeting system further comprises a nuclease function. Non-limiting examples of programmable targeting systems include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain that can be linked to a nuclease domain. Other suitable programmable targeting systems will be recognized by individuals skilled in the art.

In some aspects, the programmable targeting system is a programmable nucleic acid editing system. Such editing systems can be engineered to edit specific DNA or RNA sequences to repress transcription or translation of an mRNA encoded by the gene, and/or produce mutant proteins with reduced activity or stability. Non-limiting examples of programmable targeting nucleases include, without limit, an RNA-guided clustered regularly interspersed short palindromic repeats (CRISPR) system, such as a CRISPR-associated (Cas) (CRISPR/Cas) nuclease system, a CRISPR/Cpf1 nuclease system, a zinc finger nuclease (ZFN) system, a transcription activator-like effector nuclease (TALEN) system, a MegaTAL, a homing endonuclease (HE), a meganuclease, a ribozyme, or a programmable DNA binding domain linked to a nuclease domain. Other suitable programmable targeting nucleases will be recognized by individuals skilled in the art. Such systems rely for specificity on the delivery of exogenous protein(s), and/or a guide RNA (gRNA) or single guide RNA (sgRNA) having a sequence which binds specifically to a target nucleic acid sequence of interest. When the programmable targeting nuclease comprises more than one component, such as a protein and a guide nucleic acid, the engineered system can be modular, in that the different components may optionally be distributed among two or more nucleic acid constructs as described herein. The components can be delivered by a plasmid or viral vector or as a synthetic oligonucleotide. More detailed descriptions of programmable nucleic acid editing systems can be as described further below.

The programmable nucleic acid-binding domain can be designed or engineered to recognize and bind different nucleic acid sequences. In some aspects, the nucleic acid-binding domain is mediated by interaction between a protein and the target nucleic acid sequence. Thus, the nucleic acid-binding domain can be programmed to bind a nucleic acid sequence of interest by protein engineering. Methods of programming a nucleic acid domain are well recognized in the art.

In other targeting systems, the nucleic acid-binding domain is mediated by a guide nucleic acid that interacts with a protein of the targeting system and the target nucleic acid sequence. In such instances, the programmable nucleic acid-binding domain can be targeted to a nucleic acid sequence of interest by designing the appropriate guide nucleic acid. Methods of designing guide nucleic acids are recognized in the art when provided with a target sequence using available tools that are capable of designing functional guide nucleic acids. It will be recognized that gRNA sequences and design of guide nucleic acids can and will vary at least depending on the particular programmable targeting system used. By way of non-limiting example, guide nucleic acids optimized by sequence for use with a Cas9 nuclease are likely to differ from guide nucleic acids optimized for use with a CPF1 nuclease, though it is also recognized that the target site location is a key factor in determining guide RNA sequences.

When a programmable targeting system comprises more than one component, such as a protein and a guide nucleic acid, the multi-component programmable targeting system can be modular, in that expression of the different components may optionally be distributed among two or more nucleic acid constructs as described herein.

In some aspects, the programmable targeting system is a CRISPR/Cas nuclease system comprising a nuclease protein and a guide RNA (gRNA). In some aspects, the targeting nuclease comprises an active nuclease domain. In other aspects, the nuclease activity of the targeting nuclease is altered to only nick or cut a single strand of the double stranded nucleic acid sequence. In other aspects, the nuclease activity of the targeting nuclease is inactivated to obtain a programmable targeting protein. In some aspects, the programmable targeting nuclease is a CRISPR/Cas system. In some aspects, the CRISPR/Cas system is a CRISPR/Cas9 system and a gRNA.

In some aspects, the Cas9 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 5. In some aspects, the Cas9 protein comprises an amino acid sequence comprising at least about 75% or more, at least about 85% or more, at least about 95% or more, or 100% sequence identity with amino acid sequence of SEQ ID NO: 5.

In some aspects, a nucleic acid sequence encoding the Cas9 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. In some aspects, a nucleic acid sequence encoding the Cas9 protein comprises at least about 75% or more, at least about 85% or more, at least about 95% or more, or 100% sequence identity with the nucleic acid sequence of SEQ ID NO: 6.

In some aspects, a nucleic acid sequence encoding the Cas9 nuclease is a deCas9 nickase, and a nucleic acid expression construct for expressing the deCas9 nickase comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 89. In some aspects, a nucleic acid sequence encoding the Cas9 nuclease is a deCas9 nickase, and a nucleic acid expression construct for expressing the deCas9 nickase comprises a nucleic acid sequence comprises at least about 75% or more, at least about 85% or more, at least about 95% or more, or 100% sequence identity with the nucleic acid sequence starting at nucleotide 8218 to nucleotide 13856 of SEQ ID NO: 89.

In some aspects, the gRNA comprises a nucleic acid sequence of SEQ ID NO: 65, SEQ ID NO: 66, SEQ ID NO: 67, SEQ ID NO: 80, SEQ ID NO: 113, SEQ ID NO: 67 and SEQ ID NO: 113, or any combination thereof.

In some aspects, the targeting nuclease is not linked to the transposase. In some aspects, the engineered system comprises a nucleic acid expression construct for expressing a Pong ORF1 protein, a nucleic acid nucleic acid expression construct for expressing a Pong ORF2 protein, and a nucleic acid nucleic acid expression construct for expressing a Cas9 nuclease protein. Pong ORF1 protein, Pong ORF2 protein can be as described in Section I(a) herein above, and expression constructs for expressing Pong ORF1 and ORF2 proteins can be as described in Section II herein below.

In other aspects, a transposase of the instant disclosure is linked to the programmable targeting nuclease. In some aspects, the engineered system comprises a nucleic acid nucleic acid expression construct for expressing a Pong ORF1 protein and a nucleic acid nucleic acid expression construct for expressing a Pong ORF2 protein linked to Cas9 nuclease.

Multiple useful methods of linking proteins are known in the art and included herein. For instance, the targeting nuclease can be linked to the transposase by at least one peptide linker. Protein linkers aid fusion protein design by providing appropriate spacing between domains, supporting correct protein folding in the case that N or C termini interactions are crucial to folding. Commonly, protein linkers permit important domain interactions, reinforce stability, and reduce steric hindrance, making them preferred for use in fusion protein design even when N and C termini can be linked. Linkers can be flexible (e.g., comprising small, non-polar (e.g., Gly) or polar (e.g., Ser, Thr) amino acids). Rigid linkers can be formed of large, cyclic proline residues, which can be helpful when highly specific spacing between domains must be maintained. In vivo cleavable linkers are designed to allow the release of one or more linked domains under certain reaction conditions, such as a specific pH gradient, or when coming in contact with another biomolecule in the cell. Examples of suitable linkers are well known in the art, and programs to design linkers are readily available (Crasto et al., Protein Eng., 2000, 13 (5): 3096-312), the disclosure of which is incorporated herein in its entirety. Non-limiting examples of suitable linkers include GGSGGGSG (SEQ ID NO: 68), GSSSS (G4S; SEQ ID NO: 64) and (GGGGS) 1-4 (SEQ ID NO: 69). One or more copies of this linker may be used sequentially to create longer linkers between the tethered proteins. In some aspects, the linker is three GSSSS (SEQ ID NO: 64) linkers used sequentially to create a longer linker. Alternatively, the linker may be rigid, such as AEAAAKEAAAKA (SEQ ID NO: 70), AEAAAKEAAAKEAAAKA (SEQ ID NO: 71), PAPAP (AP) 6-8 (SEQ ID NO: 72), GIHGVPAA (SEQ ID NO: 73), EAAAK (SEQ ID NO: 76), EAAAKEAAAK (SEQ ID NO: 77), EAAAK EAAAK EAAAK (SEQ ID NO: 78), and EAAAKEAAAKEAAAKEAAAK (SEQ ID NO: 79). Other examples of suitable linkers are well known in the art, and programs to design linkers are readily available (Crasto et al., Protein Eng., 2000, 13 (5): 3096-312). In alternate aspects, the targeting nuclease and the transposase can be linked directly.

In some aspects, a transposase of the instant disclosure is linked to the programmable targeting nuclease by linking a Pong ORF2 protein to a Cas9 targeting nuclease. In some aspects, the Pong ORF2 protein is linked to a Cas9 targeting nuclease by one or more copies of a G4S linker. In some aspects, the Pong ORF2 protein is linked to a Cas9 targeting nuclease by one copy of a G4S linker. In some aspects, the Pong ORF2 protein linked to a Cas9 targeting nuclease by one copy of a G4S linker comprises an amino acid sequence encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 106. In some aspects, the Pong ORF2 protein linked to a Cas9 targeting nuclease by one copy of a G4S linker comprises an amino acid sequence encoded by a nucleic acid sequence comprising about 75% or more, at least about 85% or more, at least about 95% or more, or 100% sequence identity with the nucleic acid sequence of SEQ ID NO: 106.

In some aspects, the Pong ORF2 protein is linked to a Cas9 targeting nuclease by three copies of a G4S linker. In some aspects, the Pong ORF2 protein is linked to a Cas9 targeting nuclease by three copies of a G4S linker. In some aspects, the Pong ORF2 protein linked to a Cas9 targeting nuclease by three copies of a G4S linker comprises an amino acid sequence encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 107. In some aspects, the Pong ORF2 protein linked to a Cas9 targeting nuclease by three copies of a G4S linker comprises an amino acid sequence encoded by a nucleic acid sequence comprising about 75% or more, at least about 85% or more, at least about 95% or more, or 100% sequence identity with the nucleic acid sequence of SEQ ID NO: 107.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ~20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system may be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system may be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises a RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and an HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target nucleic acid loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

A nuclease of a CRISPR nuclease system can be inactivated to obtain a programmable targeting protein. For instance, a CRISPR/Cas system can comprise a nuclease-deficient dead CAS9 protein (dCAS9) and a guide RNA (gRNA).

ii. CRISPR Nickase Systems.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. ssDNA-Guided Argonaute Systems.

Alternatively, the programmable targeting nuclease may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

iv. Zinc Finger Nucleases.

The programmable targeting nuclease may be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include Bfil, Bpml, Bsal, Bsgl, BsmBl, Bsml, BspMl, Fokl, MboII, and SapI. The type II-S nuclease domain may be modified to facilitate dimerization of two different nuclease domains. For example, the cleavage domain of Fokl may be modified by mutating certain amino acid residues. By way of non-limiting example, amino acid residues at positions 446, 447, 479, 483, 484, 486, 487, 490, 491, 496, 498, 499, 500, 531, 534, 537, and 538 of Fokl nuclease domains are targets for modification. For example, one modified Fokl domain may comprise Q486E, I499L, and/or N496D mutations, and the other modified Fokl domain may comprise E490K, I538K, and/or H537R mutations.

v. Transcription Activator-Like Effector Nuclease Systems.

The programmable targeting nuclease may also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section (I) (c) (i).

vi. Meganucleases or Rare-Cutting Endonuclease Systems

The programmable targeting nuclease may also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that may be suitable for the instant disclosure include I-Scel, I-Crel, I-Dmol, or variants and combinations thereof. A meganuclease may be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease may recognize a 7-nucleotide sequence, an 8-nucleotide sequence, or longer recognition sequence. Non-limiting examples of rare-cutting endonucleases include Notl, Ascl, Pacl, AsiSl, Sbfl, and Fsel.

vii. Optional Additional Domains.

The programmable targeting nuclease may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS may be located at the N-terminus, the C-terminal, or in an internal location of the fusion protein.

A cell-penetrating domain may be a cell-penetrating peptide sequence derived from the HIV-1 TAT protein. The cell-penetrating domain may be located at the N-terminus, the C-terminal, or in an internal location of the fusion protein.

A programmable targeting nuclease may further comprise at least one linker. For example, the programmable targeting nuclease, the nuclease domain of the targeting nuclease, and other optional domains may be linked via one or more linkers. The linker may be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13 (5): 3096-312). In alternate aspects, the programmable targeting nuclease, the cell cycle regulated protein, and other optional domains may be linked directly.

A programmable targeting nuclease may further comprise an organelle localization or targeting signal that directs a molecule to a specific organelle. A signal may be polynucleotide or polypeptide signal, or may be an organic or inorganic compound sufficient to direct an attached molecule to a desired organelle. Organelle localization signals can be as described in U.S. Patent Publication No. 20070196334, the disclosure of which is incorporated herein in its entirety.

### (d) Engineered System

An engineered system of the instant disclosure generally comprises a nucleic acid expression construct for expressing a tranposase, wherein the expression construct comprises a promoter operably linked to a nucleic acid sequence encoding a transposase. The engineered system also comprises a donor polynucleotide comprising nucleic acid transposition sequences compatible with the transposase and a nucleic acid expression construct for expressing a programmable targeting system, wherein the expression construct comprises a promoter operably linked to a nucleic acid sequence encoding a programmable targeting system. The programmable targeting system is programmed to target the transposase and the donor polynucleotide to a target nucleic acid locus in the cell, thereby accomplishing insertion of the donor polynucleotide at the target nucleic acid locus to generate a genetically modified cell comprising the donor polynucleotide inserted at the target nucleic acid locus.

In some aspects, the targeting system comprises a targeting nuclease and is engineered to introduce a cut in a target nucleic acid locus. In other aspects, the targeting system does not comprise a nuclease function. The transposase can be linked to the targeting system. Alternatively, the transposase is not linked to the targeting nuclease.

The system can further comprise a nucleic acid expression construct comprising a promoter operably linked to a polynucleotide sequence encoding a reporter, wherein the donor polynucleotide is inserted in the nucleic acid expression construct, wherein the reporter is inactivated by the inserted nucleic acid construct comprising the donor polynucleotide, and wherein the reporter is activated by excision of the inserted nucleic acid construct comprising the donor polynucleotide from the expression construct comprising a promoter operably linked to a polynucleotide sequence encoding a reporter by the transposase. In some aspects, the reporter can be GFP, and the GFP expression construct, wherein the donor polynucleotide is inserted in the nucleic acid expression construct, comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the reporter can be GFP, and the GFP expression construct, wherein the donor polynucleotide is inserted in the nucleic acid expression construct, comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

The transposase can be a split transposase. When the transposase is a split transposase, the transposase can be a Pong or Pong-like transposase comprising a Pong ORF1 protein and a Pong ORF2 protein. In some aspects, the Pong ORF1 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. In some aspects, the Pong ORF1 protein comprises an amino acid sequence comprising at least about 75% or more, at least about 85% or more, at least about 95% or more, or 100% sequence identity with the amino acid sequence of SEQ ID NO: 1. A nucleic acid sequence encoding the Pong ORF1 protein can comprise about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 2. A nucleic acid sequence encoding the Pong ORF1 protein can comprise at least about 75% or more, at least about 85% or more, at least about 95% or more, or 100% sequence identity with the nucleic acid sequence of SEQ ID NO: 2.

In some aspects, the Pong ORF2 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 3. In some aspects, the Pong ORF2 protein comprises an amino acid sequence comprising at least about 75% or more, at least about 85% or more, at least about 95% or more, or 100% sequence identity with the amino acid sequence of SEQ ID NO: 3. A nucleic acid sequence encoding the Pong ORF2 protein can comprise about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 4. A nucleic acid sequence encoding the Pong ORF2 protein can comprise at least about 75% or more, at least about 85% or more, at least about 95% or more, or 100% sequence identity with the amino acid sequence of SEQ ID NO: 4.

The transposition sequences can be transposition sequences of a miniature inverted-repeat transposable element (MITE). In some aspects, the MITE is an mPing MITE or a derivative of mPing with sequences added or removed. In some aspects, transposition sequences of the mPing MITE comprise mPing inverted repeat 1 and inverted repeat 2. In some aspects, mPing inverted repeat 1 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7, SEQ ID NO: 111, or SEQ ID NO: 108. In some aspects, mPing inverted repeat 1 comprises a nucleotide sequence comprising at least about 75% or more, at least about 85% or more, at least about 95% or more, or 100% sequence identity with the nucleic acid sequence of SEQ ID NO: 7, SEQ ID NO: 111, or SEQ ID NO: 108. In some aspects, mPing inverted repeat 2 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 8, SEQ ID NO: 112, or SEQ ID NO: 109. In some aspects, mPing inverted repeat 2 comprises a nucleotide sequence comprising at least about 75% or more, at least about 85% or more, at least about 95% or more, or 100% sequence identity with the nucleic acid sequence of SEQ ID NO: 8, SEQ ID NO: 112, or SEQ ID NO: 109.

The system comprises an expression construct for expressing the Pong ORF1 protein, wherein the expression construct for expressing the Pong ORF1 protein can comprise at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 100. In some aspects, the expression construct for expressing the Pong ORF1 protein comprises at least about 75% or more, at least about 85% or more, at least about 95% or more, or 100% sequence identity with SEQ ID NO: 100.

The programmable targeting system can be a CRISPR/Cas system comprising a Cas9 nuclease and a guide RNA (gRNA). In some aspects, the Cas9 nuclease comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 5. In some aspects, the Cas9 nuclease comprises an amino acid sequence comprising at least about 75% or more, at least about 85% or more, at least about 95% or more, or 100% sequence identity with the amino acid sequence of SEQ ID NO: 5.

In some aspects, the Cas9 nuclease is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. In some aspects, the Cas9 nuclease is encoded by a nucleic acid sequence comprising at least about 75% or more, at least about 85% or more, at least about 95% or more, or 100% sequence identity with the nucleic acid sequence of SEQ ID NO: 6. In some aspects, the gRNA comprises a nucleic acid sequence of SEQ ID NO: 65, SEQ ID NO: 66, SEQ ID NO: 67, SEQ ID NO: 80, SEQ ID NO: 113, SEQ ID NO: 67 and SEQ ID NO: 113, or any combination thereof.

The transposase can be linked to the Cas9 nuclease. When the transposase is linked to the Cas9 nuclease, an engineered system of the instant disclosure comprises a Pong ORF2 protein is linked to the Cas9 nuclease by one copy of a G4S linker of SEQ ID NO: 64. In some aspects, the Pong ORF2 protein linked to the Cas9 nuclease by one copy of a G4S linker of SEQ ID NO: 64 comprises an amino acid sequence encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 106 or a nucleic acid sequence starting at base 8392 to base 14052 of SEQ ID NO: 74. In some aspects, the Pong ORF2 protein linked to the Cas9 nuclease by one copy of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 106 or a nucleic acid sequence starting at base 8392 to base 14052 of SEQ ID NO: 74.

In some aspects, the engineered system comprises an expression construct for expressing the Pong ORF2 protein linked to the Cas9 nuclease by one copy of a G4S linker of SEQ ID NO: 64, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence starting at base 7451 to base 15799 of SEQ ID NO: 74. In some aspects, the engineered system comprises an expression construct for expressing the Pong ORF2 protein linked to the Cas9 nuclease by one copy of a G4S linker of SEQ ID NO: 64, wherein the expression construct comprises a nucleic acid sequence comprising at least about 75% or more, at least about 85% or more, at least about 95% or more, or 100% sequence identity with a nucleic acid sequence starting at base 7451 to base 15799 of SEQ ID NO: 74. In some aspects, the cell is an Arabidopsis thaliana cell.

In some aspects, the programmable targeting system of the instant disclosure comprises a CRISPR nuclease system comprising dCas9 and a gRNA. In some aspects, the dCas9 nuclease is linked to Pong ORF2 by one copy of a G4S linker of SEQ ID NO: 64. In some aspects, the Pong ORF2 protein linked to the dCas9 nuclease by one copy of a G4S linker of SEQ ID NO: 64 comprises an amino acid sequence encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 110. In some aspects, the Pong ORF2 protein linked to the dCas9 nuclease by one copy of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 110.

In some aspects, the Pong ORF2 protein linked to the dCas9 nuclease by one copy of a G4S linker of SEQ ID NO: 64 is expressed using an expression construct for expressing the Pong ORF2 protein linked to the dCas9 nuclease by one copy of a G4S linker of SEQ ID NO: 64, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 115. In some aspects, the expression construct comprises a nucleic acid sequence comprising at least about 75% or more, at least about 85% or more, at least about 95% or more, or 100% sequence identity with SEQ ID NO: 115. In some aspects, the genetically modified cell is an Arabidopsis thaliana cell.

In some aspects, the dCas9 nuclease is linked to Pong ORF2 by three copies of a G4S linker of SEQ ID NO: 64. In some aspects, the Pong ORF2 protein linked to the dCas9 nuclease by three copies of a G4S linker of SEQ ID NO: 64 comprises an amino acid sequence encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 107. In some aspects, the Pong ORF2 protein linked to the dCas9 nuclease by one copy of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 107.

In some aspects, the Pong ORF2 protein linked to the Cas9 nuclease by three copies of a G4S linker of SEQ ID NO: 64 is expressed using an expression construct for expressing the Pong ORF2 protein linked to the Cas9 nuclease by three copies of a G4S linker of SEQ ID NO: 64, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 104. In some aspects, the expression construct comprises a nucleic acid sequence comprising at least about 75% or more, at least about 85% or more, at least about 95% or more, or 100% sequence identity with SEQ ID NO: 104. In some aspects, the genetically modified cell is a soybean cell.

In some aspects, the Pong ORF2 protein is not linked to the targeting nuclease. When the Pong ORF2 protein is not linked to the targeting nuclease, the engineered system can comprise a nucleic acid expression construct for expressing a Cas9 nuclease, wherein the expression construct for expressing the Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 92 or a nucleic acid sequence starting at base 10857 to base 16495 of SEQ ID NO: 94. In some aspects, the expression construct for expressing the Cas9 nuclease comprises a nucleic acid sequence comprising at least about 75% or more, at least about 85% or more, at least about 95% or more, or 100% sequence identity with SEQ ID NO: 92 or a nucleic acid sequence starting at base 10857 to base 16495 of SEQ ID NO: 94.

When the Pong ORF2 protein is not linked to the targeting nuclease, the engineered system can comprise a nucleic acid expression construct for expressing a Pong ORF2 protein, wherein the expression construct for expressing the Pong ORF2 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO 101 or a nucleic acid sequence starting at base 5073 to base 8215 of SEQ ID NO: 89. In some aspects, the expression construct for expressing the Pong ORF2 protein comprises a nucleic acid sequence comprising at least about 75% or more, at least about 85% or more, at least about 95% or more, or 100% sequence identity with a nucleic acid sequence of SEQ ID NO 101 or a nucleic acid sequence starting at base 5073 to base 8215 of SEQ ID NO: 89.

The first mPing transposition sequence and the second mPing transposition sequence can flank a cargo polynucleotide. In some aspects, the cargo polynucleotide comprises HSEs. When the cargo polynucleotide comprises HSEs, the first mPing transposition sequence can comprise at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7 and the second mPing transposition sequence can comprise at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 8. In some aspects, the first mPing transposition sequence comprises at least about 75% or more, at least about 85% or more, at least about 95% or more, or 100% sequence identity with the nucleic acid sequence of SEQ ID NO: 7 and wherein the second mPing transposition sequence comprises at least about 75% or more, at least about 85% or more, at least about 95% or more, or 100% sequence identity with the nucleic acid sequence of SEQ ID NO: 8. In some aspects, the donor polynucleotide comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 81. In some aspects, the donor polynucleotide comprises at least about 75% or more, at least about 85% or more, at least about 95% or more, or 100% sequence identity with the nucleic acid sequence of SEQ ID NO: 81.

In some aspects, the cargo polynucleotide comprises an expression construct for expressing a herbicide resistance function. The herbicide resistance function can be resistance to bialaphos herbicide. When the herbicide resistance function can be resistance to bialaphos herbicide, the first mPing transposition sequence can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 108 and the second mPing transposition sequence can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 109. In some aspects, the first mPing transposition sequence comprises a nucleic acid sequence comprising at least about 75% or more, at least about 85% or more, at least about 95% or more, or 100% sequence identity with SEQ ID NO: 108 and the second mPing transposition sequence comprises a nucleic acid sequence comprising at least about 75% or more, at least about 85% or more, at least about 95% or more, or 100% sequence identity with SEQ ID NO: 109.

In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 97 or SEQ ID NO: 99. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97 or SEQ ID NO: 99.

In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 97. In some aspects, the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97.

In some aspects, the engineered system comprises an expression construct for expressing a gRNA for targeting the transposase and nuclease to a target nucleic acid locus in an Arabidopsis thaliana PDS3 gene, wherein the expression construct for expressing the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 2632 to base 3343 of SEQ ID NO: 74. In some aspects, the expression construct for expressing the gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74.

In some aspects, the engineered system comprises an expression construct for expressing a gRNA for targeting the transposase and nuclease to a target nucleic acid locus in an Arabidopsis thaliana ADH1 gene, wherein the expression construct for expressing a gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 89. In some aspects, the expression construct for expressing a gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89.

In some aspects, the engineered system comprises an expression construct for expressing a gRNA for targeting the transposase and nuclease to a target nucleic acid locus in an Arabidopsis thaliana ACT8 gene, wherein the expression construct for expressing a gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 103 or the nucleic acid sequence starting at base 729 to base 1440 of SEQ ID NO: 92. In some aspects, the expression construct for expressing a gRNA comprises a nucleic acid sequence comprising at least about 75% or more, at least about 85% or more, at least about 95% or more, or 100% sequence identity with the nucleic acid sequence of SEQ ID NO: 103 or the nucleic acid sequence starting at base 729 to base 1440 of SEQ ID NO: 92.

In some aspects, the engineered system comprises an expression construct for expressing a gRNA for targeting the transposase and nuclease to a target nucleic acid locus in a soybean DD20 intergenic region, wherein the expression construct for expressing a gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 105. In some aspects, the expression construct for expressing a gRNA comprises a nucleic acid sequence comprising at least about 75% or more, at least about 85% or more, at least about 95% or more, or 100% sequence identity with the nucleic acid sequence of SEQ ID NO: 105.

Another aspect of the instant disclosure encompasses an engineered system for generating a genetically modified cell, wherein the engineered system comprises

In some aspects, the system comprises a nucleic acid expression construct for expressing a Pong ORF1 protein, wherein the expression construct for expressing a Pong ORF1 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100; a nucleic acid expression construct for expressing a Pong ORF2 protein linked to Cas9 nuclease with one copy of a G4S linker, wherein the expression construct for expressing the Pong ORF2 protein linked to Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 7451 to base 14807 of SEQ ID NO: 74; a donor polynucleotide comprising first and second mPing transposition sequences; and an expression construct for expressing a gRNA, wherein the expression construct for expressing the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 103. In some aspects, the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100. In some aspects, the expression construct for expressing the Pong ORF2 protein linked to Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74. In some aspects, the expression construct for expressing the gRNA comprises a nucleic acid sequence comprising at least about 75% or more, at least about 85% or more, at least about 95% or more, or 100% sequence identity with the nucleic acid sequence of SEQ ID NO: 103.

In some aspects, the donor polynucleotide comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 81. In some aspects, the donor polynucleotide comprises at least about 75% or more, at least about 85% or more, at least about 95% or more, or 100% sequence identity with the nucleic acid sequence of SEQ ID NO: 81.

In some aspects, the system comprises a nucleic acid expression construct for expressing a Pong ORF1 protein, wherein the expression construct for expressing a Pong ORF1 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100; a nucleic acid nucleic acid expression construct for expressing a Pong ORF2 protein, wherein the expression construct for expressing the Pong ORF2 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 101; a nucleic acid nucleic acid expression construct for expressing a Cas9 nuclease, wherein the expression construct for expressing the Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 102; a donor polynucleotide comprising first and second mPing transposition sequences; and an expression construct for expressing a gRNA, wherein the expression construct for expressing the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 103. In some aspects, the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101. In some aspects, the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102. In some aspects, the expression construct for expressing the gRNA comprises a nucleic acid sequence comprising at least about 75% or more, at least about 85% or more, at least about 95% or more, or 100% sequence identity with the nucleic acid sequence of SEQ ID NO: 103.

The donor polynucleotide comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 81. In some aspects, the donor polynucleotide comprises at least about 75% or more, at least about 85% or more, at least about 95% or more, or 100% sequence identity with the nucleic acid sequence of SEQ ID NO: 81.

In some aspects, the engineered system comprises a nucleic acid expression construct for expressing a Pong ORF1 protein, wherein the expression construct for expressing a Pong ORF1 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100; a nucleic acid nucleic acid expression construct for expressing a Pong ORF2 protein, wherein the expression construct for expressing the Pong ORF2 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 101; a nucleic acid nucleic acid expression construct for expressing a Cas9 nuclease, wherein the expression construct for expressing the Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 102; a donor polynucleotide comprising first and second mPing transposition sequences; and an expression construct for expressing a gRNA, wherein the expression construct for expressing the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 103. In some aspects, the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101. In some aspects, the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102. In some aspects, the expression construct for expressing the gRNA comprises a nucleic acid sequence comprising at least about 75% or more, at least about 85% or more, at least about 95% or more, or 100% sequence identity with the nucleic acid sequence of SEQ ID NO: 103.

The donor polynucleotide comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 81. In some aspects, the donor polynucleotide comprises at least about 75% or more, at least about 85% or more, at least about 95% or more, or 100% sequence identity with the nucleic acid sequence of SEQ ID NO: 81.

In some aspects, the system comprises a nucleic acid expression construct for expressing a Pong ORF1 protein, wherein the expression construct for expressing a Pong ORF1 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100; a nucleic acid nucleic acid expression construct for expressing a Pong ORF2 protein, wherein the expression construct for expressing the Pong ORF2 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 101; a nucleic acid nucleic acid expression construct for expressing a Cas9 nuclease, wherein the expression construct for expressing the Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 102; a donor polynucleotide comprising first and second mPing transposition sequences; and an expression construct for expressing a gRNA, wherein the expression construct for expressing the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 105. In some aspects, the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101. In some aspects, the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102. In some aspects, the expression construct for expressing the gRNA comprises a nucleic acid sequence comprising at least about 75% or more, at least about 85% or more, at least about 95% or more, or 100% sequence identity with the nucleic acid sequence of SEQ ID NO: 105.

In some aspects, the donor polynucleotide comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 81. In some aspects, the donor polynucleotide comprises at least about 75% or more, at least about 85% or more, at least about 95% or more, or 100% sequence identity with the nucleic acid sequence of SEQ ID NO: 81.

In some aspects, the engineered system comprises a nucleic acid expression construct for expressing a Pong ORF1 protein, wherein the expression construct for expressing a Pong ORF1 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100; a nucleic acid nucleic acid expression construct for expressing a Pong ORF2 protein, wherein the expression construct for expressing the Pong ORF2 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 101; a nucleic acid nucleic acid expression construct for expressing a Cas9 nuclease, wherein the expression construct for expressing the Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 102; a donor polynucleotide comprising first and second mPing transposition sequences; and an expression construct for expressing a gRNA of SEQ ID NO: 67 and a gRNA of SEQ ID NO: 113, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 114. In some aspects, the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence of SEQ ID NO: 101. In some aspects, the expression construct for expressing the Cas9 nuclease comprises a nucleic acid sequence comprising at least about 75% or more, at least about 85% or more, at least about 95% or more, or 100% sequence identity with the nucleic acid sequence of SEQ ID NO: 102. In some aspects, the expression construct comprises a nucleic acid sequence comprising at least about 75% or more, at least about 85% or more, at least about 95% or more, or 100% sequence identity with the nucleic acid sequence of SEQ ID NO: 114.

In some aspects, the system comprises a nucleic acid expression construct for expressing a Pong ORF1 protein, wherein the expression construct for expressing a Pong ORF1 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100; a nucleic acid nucleic acid expression construct for expressing a Pong ORF2 protein linked to dCas9 nuclease with one copy of a G4S linker, wherein the expression construct for expressing the Pong ORF2 protein linked to Cas9 nuclease comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 115; a donor polynucleotide comprising first and second mPing transposition sequences; and an expression construct for expressing a gRNA of SEQ ID NO: 67 and a gRNA of SEQ ID NO: 113, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 114. In some aspects, the expression construct for expressing a Pong ORF1 protein comprises a nucleic acid sequence comprising at least about 75% or more, at least about 85% or more, at least about 95% or more, or 100% sequence identity with the nucleic acid sequence of SEQ ID NO: 100. In some aspects, the expression construct for expressing the Pong ORF2 protein linked to Cas9 nuclease comprises a nucleic acid sequence comprising at least about 75% or more, at least about 85% or more, at least about 95% or more, or 100% sequence identity with the nucleic acid sequence of SEQ ID NO: 115. In some aspects, the expression construct comprises a nucleic acid sequence comprising at least about 75% or more, at least about 85% or more, at least about 95% or more, or 100% sequence identity with the nucleic acid sequence of SEQ ID NO: 114.

As explained in Section II further below, a system of the instant disclosure can be encoded on one or more nucleic acid constructs encoding the components of the system. Depending on an intended use of the system of the instant disclosure, the number of nucleic acid constructs encoding the components of the system can be on different plasmids based on intended use. For instance, the systems can be a one-component system comprising all the elements of the system. Such a system can provide the convenience and simplicity of introducing a single nucleic acid construct into a cell.

In some aspects, an engineered system of the instant disclosure comprises a Pong transposase, wherein the nucleic acid transposition sequences are mPing inverted repeat 1 and inverted repeat 2, and the programmable targeting nuclease comprises a Cas9 nuclease and a gRNA. In some aspects, the Pong ORF2 protein is linked to the Cas9 nuclease. In some aspects, the Pong ORF2 protein is not linked to the Cas9 nuclease.

In some aspects, an engineered system of the instant disclosure comprises a donor polynucleotide comprising a first and second mPing miniature inverted-repeat transposable element (MITE) transposition sequences; one or more nucleic acid expression constructs for expressing a tranposase comprising a Pong ORF1 protein and a Pong ORF2 protein, wherein each of the one or more expression constructs comprises a promoter operably linked to a nucleic acid sequence encoding the Pong ORF1 protein and the Pong ORF2 protein; and a nucleic acid expression construct for expressing a programmable targeting system, wherein the expression construct comprises a promoter operably linked to a nucleic acid sequence encoding the programmable targeting system. The programmable targeting system is programmed to target the transposase and the donor polynucleotide to a target nucleic acid locus in the cell, to introduce a cut in the target nucleic acid locus, or both, thereby accomplishing insertion of the donor polynucleotide at the target nucleic acid locus to generate a genetically modified cell comprising the donor polynucleotide inserted at the target nucleic acid locus.

In some aspects, the system further comprises a reporter nucleic acid construct for expressing a reporter, wherein the reporter nucleic acid construct comprises a promoter operably linked to a polynucleotide sequence encoding the reporter, wherein the donor polynucleotide is inserted in the reporter nucleic acid construct thereby inactivating expression of the reporter, and wherein expression of the reporter is activated by excision of the inserted donor polynucleotide from the reporter nucleic acid construct by the transposase. In some aspects, the reporter is GFP, and the nucleic acid expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the reporter is GFP, and the nucleic acid expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

A system of the instant disclosure can be encoded on more than one nucleic acid construct. In some aspects, a system of the instant disclosure comprises a two-component system comprising a donor nucleic acid construct comprising the nucleic acid construct comprising a donor polynucleotide of the instant disclosure, and a helper nucleic acid construct comprising a nucleic acid expression construct for expressing a transposase and the nucleic acid expression construct for expressing the programmable targeting nuclease of the instant disclosure.

The system of any of the preceding disclosure, wherein the cell is a plant cell, a plant or part thereof, or seed.

### II. Nucleic Acid Constructs

A further aspect of the present disclosure provides one or more nucleic acid constructs encoding the components of the engineered system described above in Section I. In some aspects, the engineered system of nucleic acid constructs encodes the engineered system described in Section I(d).

Any of the multi-component engineered systems described herein are to be considered modular, in that the different components may optionally be distributed among two or more nucleic acid constructs as described herein. The nucleic acid constructs may be DNA or RNA, linear or circular, single-stranded or double-stranded, or any combination thereof. The nucleic acid constructs may be codon optimized for efficient translation into protein, and possibly for transcription into an RNA donor polynucleotide transcript in the cell of interest. Codon optimization programs are available as freeware or from commercial sources.

The nucleic acid constructs can be used to express one or more components of the engineered system for later introduction into a cell to be genetically modified. Alternatively, the nucleic acid constructs can be introduced into the cell to be genetically modified for expression of the components of the engineered system in the cell.

Expression constructs generally comprise DNA coding sequences operably linked to at least one promoter control sequence for expression in a cell of interest. Promoter control sequences may control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

Promoters may also be plant-specific promoters, or promoters that may be used in plants. A wide variety of plant promoters are known to those of ordinary skill in the art, as are other regulatory elements that may be used alone or in combination with promoters. Preferably, promoter control sequences control expression in cassava such as promoters disclosed in Wilson et al., 2017, The New Phytologist, 213 (4): 1632-1641, the disclosure of which is incorporated herein in its entirety.

Promoters may be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible-promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604, 121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters may include, but are not limited to, fiber-specific, green tissue-specific, root-specific, stem-specific, flower-specific, callus-specific, pollen-specific, egg-specific, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262:12202, 1987; Baszczynski et al., Plant Mol. Biol. 14:633, 1990), Brazil Nut albumin (Pearson et al., Plant Mol. Biol. 18:235-245, 1992), legumin (Ellis et al., Plant Mol. Biol. 10:203-214, 1988), Glutelin (rice) (Takaiwa et al., Mol. Gen. Genet. 208:15-22, 1986; Takaiwa et al., FEBS Letts. 221:43-47, 1987), Zein (Matzke et al., Plant Mol Biol, 143:323-32, 1990), napA (Stalberg et al., Planta 199:515-519, 1996), Wheat SPA (Albanietal, Plant Cell, 9:171-184, 1997), sunflower oleosin (Cummins et al., Plant Mol. Biol. 19:873-876, 1992)], endosperm specific promoters [e.g., wheat LMW and HMW, glutenin-1 (Mol Gen Genet 216:81-90, 1989; NAR 17:461-2), wheat a, b and g gliadins (EMBO3: 1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116 (1): 53-62, 1998), Biz2 (EP99106056.7), Synthetic promoter (Vicente-Carbajosa et al., Plant J. 13:629-640, 1998), rice prolamin NRP33, rice-globulin Glb-1 (Wu et al., Plant Cell Physiology 39 (8) 885-889, 1998), rice alpha-globulin REB/OHP-1 (Nakase et al., Plant Mol. Biol. 33:513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93:8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Any of the promoter sequences may be wild type or may be modified for more efficient or efficacious expression. The DNA coding sequence also may be linked to a polyadenylation signal (e.g., SV40 polyA signal, bovine growth hormone (BGH) polyA signal, etc.) and/or at least one transcriptional termination sequence. In some situations, the complex or fusion protein may be purified from the bacterial or eukaryotic cells.

Nucleic acids encoding one or more components of an engineered system of the instant disclosure can be present in a construct. Suitable constructs include plasmid constructs, viral constructs, and self-replicating RNA (Yoshioka et al., Cell Stem Cell, 2013, 13:246-254). For instance, the nucleic acid encoding one or more components of an engineered system of the instant disclosure can be present in a plasmid construct.

Non-limiting examples of suitable plasmid constructs include pUC, pBR322, pET, pBluescript, and variants thereof. Alternatively, the nucleic acid encoding one or more components of an engineered system of the instant disclosure can be part of a viral vector (e.g., lentiviral vectors, adeno-associated viral vectors, adenoviral vectors, and so forth).

The plasmid or viral vector can comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector may further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector may further comprise sequences for expression of Csy4 RNAse to process the gRNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

In some aspects, a nucleic acid construct of the instant disclosure comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100. In some aspects, the nucleic acid expression construct for expressing a Pong ORF1 protein comprises at least about 75% or more, at least about 85% or more, at least about 95% or more, or 100% sequence identity with the nucleic acid sequence of SEQ ID NO: 100.

In some aspects, a nucleic acid construct of the instant disclosure comprises a nucleic acid expression construct for expressing a Pong ORF2 protein, wherein the expression construct for expressing the Pong ORF2 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 101. In some aspects, the nucleic acid expression construct for expressing a Pong ORF2 protein comprises at least about 75% or more, at least about 85% or more, at least about 95% or more, or 100% sequence identity with the nucleic acid sequence of SEQ ID NO: 101.

In some aspects, a nucleic acid construct of the instant disclosure comprises a nucleic acid expression construct for expressing a Cas9 protein, wherein the expression construct for expressing the Cas9 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 102. In some aspects, the nucleic acid expression construct for expressing a Cas9 protein comprises at least about 75% or more, at least about 85% or more, at least about 95% or more, or 100% sequence identity with the nucleic acid sequence of SEQ ID NO: 102.

In some aspects, a nucleic acid construct of the instant disclosure comprises a nucleic acid expression construct for expressing a gRNA for targeting a transposase and nuclease to the DD20 intergenic region of soybean, wherein the expression construct for expressing the gRNA for targeting a transposase and nuclease of the instant disclosure to the DD20 intergenic region of soybean comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 105. In some aspects, the nucleic acid expression construct for expressing a gRNA directed to the DD20 intergenic region of soybean comprises at least about 75% or more, at least about 85% or more, at least about 95% or more, or 100% sequence identity with the nucleic acid sequence of SEQ ID NO: 105.

In some aspects, a system of the instant disclosure is a one-component system, wherein the Pong ORF2 protein is linked to the Cas9 nuclease and the donor polynucleotide is inserted in a nucleic acid expression construct encoding a GFP reporter, thereby inactivating the reporter. In these aspects, the target nucleic acid locus is in an Arabidopsis PDS3 gene. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 100 or the nucleic acid sequence starting at base 5073 to base 8215 of SEQ ID NO: 89. In some aspects, the nucleic acid expression construct for expressing a Pong ORF1 protein comprises at least about 75% or more, at least about 85% or more, at least about 95% or more, or 100% sequence identity with the nucleic acid sequence of SEQ ID NO: 100 or the nucleic acid sequence starting at base 5073 to base 8215 of SEQ ID NO: 89. The system also comprises a nucleic acid expression construct for expressing a Pong ORF2 protein linked to Cas9 nuclease by a single copy of the G4S linker (SEQ ID NO: 64), wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 115 or a nucleic acid sequence starting at base 7451 to base 15799 of SEQ ID NO: 74. In some aspects, the construct for expressing a Pong ORF2 protein linked to Cas9 nuclease by a single copy of the G4S linker (SEQ ID NO: 64) comprises a nucleic acid sequence comprising at least about 75% or more, at least about 85% or more, at least about 95% or more, or 100% sequence identity with SEQ ID NO: 115 or a nucleic acid sequence starting at base 7451 to base 15799 of SEQ ID NO: 74. The system further comprises a nucleic acid expression construct comprising a promoter operably linked to a polynucleotide sequence encoding GFP, wherein the donor polynucleotide inserted in the nucleic acid expression construct. In some aspects, the GFP expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the GFP expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 2632 to base 3343 of SEQ ID NO: 74. In some aspects, the expression construct for expressing the gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 74. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 74.

In some aspects, a system of the instant disclosure is a one-component system, wherein the Pong ORF2 protein is linked to the Cas9 nuclease and the donor polynucleotide is inserted in a nucleic acid expression construct encoding a GFP reporter, thereby inactivating the reporter. In these aspects, the target nucleic acid locus is in an actin 8 (ACT8) gene. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 1456 to base 5362 of SEQ ID NO: 92. In some aspects, the nucleic acid expression construct for expressing a Pong ORF1 protein comprises at least about 75% or more, at least about 85% or more, at least about 95% or more, or 100% sequence identity with the nucleic acid sequence starting at base 1456 to base 5362 of SEQ ID NO: 92. The system also comprises a nucleic acid nucleic acid expression construct for expressing a Pong ORF2 protein linked to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 108 or the nucleic acid sequence starting at base 5548 to base 12904 of SEQ ID NO: 92. In some aspects, the construct for expressing a Pong ORF2 protein linked to Cas9 nuclease comprises a nucleic acid sequence comprising at least about 75% or more, at least about 85% or more, at least about 95% or more, or 100% sequence identity with the nucleic acid sequence of SEQ ID NO: 108 or the nucleic acid sequence starting at base 5548 to base 12904 of SEQ ID NO: 92. The system further comprises a nucleic acid construct comprising the donor polynucleotide, wherein the nucleic acid construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 498 of SEQ ID NO: 92. In some aspects, the nucleic acid construct comprising the donor polynucleotide comprises a nucleic acid sequence comprising at least about 75% or more, at least about 85% or more, at least about 95% or more, or 100% sequence identity with the nucleic acid sequence starting at base 69 to base 498 of SEQ ID NO: 92. The system comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 729 to base 1440 of SEQ ID NO: 92. In some aspects, the expression construct for expressing the gRNA comprises a nucleic acid sequence comprising at least about 75% or more, at least about 85% or more, at least about 95% or more, or 100% sequence identity with the nucleic acid sequence starting at base 729 to base 1440 of SEQ ID NO: 92. In some aspects, the system is encoded on a plasmid comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 92. In some aspects, the system is encoded on a plasmid comprising at least about 75% or more, at least about 85% or more, at least about 95% or more, or 100% sequence identity with SEQ ID NO: 92.

In other aspects, a system of the instant disclosure is a one-component system, wherein the Pong ORF2 protein linked to a Cas9 nuclease and the target nucleic acid locus is in an Arabidopsis actin 8 (ACT8) gene. In these aspects, the donor polynucleotide comprises a nucleotide sequence comprising heat shock element (HSE) sequences flanked by mPing inverted repeat 1 and inverted repeat 2. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 1481 to base 5390 of SEQ ID NO: 93. In some aspects, the nucleic acid expression construct for expressing a Pong ORF1 protein comprises at least about 75% or more, at least about 85% or more, at least about 95% or more, or 100% sequence identity with the nucleic acid sequence starting at base 1481 to base 5390 of SEQ ID NO: 93. The system also comprises a nucleic acid expression construct for expressing a Pong ORF2 protein linked to Cas9 nuclease, wherein the expression construct for expressing the Pong ORF2 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 1481 to base 5390 of SEQ ID NO: 93. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence starting at base 1481 to base 5390 of SEQ ID NO: 93. The system further comprises a nucleic acid construct comprising the donor polynucleotide, wherein the donor polynucleotide comprises a nucleotide sequence comprising HSE sequences flanked by mPing inverted repeat 1 and inverted repeat 2, and wherein the donor polynucleotide comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 512 of SEQ ID NO: 93. In some aspects, the donor polynucleotide comprises at least about 75% or more, at least about 85% or more, at least about 95% or more, or 100% sequence identity with the nucleic acid sequence starting at base 69 to base 512 of SEQ ID NO: 93. The system comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 754 to base 1465 of SEQ ID NO: 93. In some aspects, the expression construct for expressing the gRNA comprises a nucleic acid sequence comprising at least about 75% or more, at least about 85% or more, at least about 95% or more, or 100% sequence identity with the nucleic acid sequence starting at base 754 to base 1465 of SEQ ID NO: 93. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 93. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 93.

In some aspects, a system of the instant disclosure is a one-component system, wherein the Cas9 protein is not linked to the Pong ORF2 protein, and the target nucleic acid locus is in a soybean DD20 intergenic region. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with nucleic acid sequence starting at base 3593 to base 7502 of SEQ ID NO: 94. In some aspects, the nucleic acid expression construct for expressing a Pong ORF1 protein comprises at least about 75% or more, at least about 85% or more, at least about 95% or more, or 100% sequence identity with the nucleic acid sequence starting at base 3593 to base 7502 of SEQ ID NO: 94. The system also comprises a nucleic acid nucleic acid expression construct for expressing a Pong ORF2 protein, wherein the expression construct for expressing the Pong ORF2 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 7685 to base 10827 of SEQ ID NO: 94. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence starting at base 7685 to base 10827 of SEQ ID NO: 94. The system also comprises a nucleic acid expression construct for expressing a Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 10857 to base 16495 of SEQ ID NO: 94. In some aspects, the construct for expressing the Cas9 nuclease comprises a nucleic acid sequence comprising at least about 75% or more, at least about 85% or more, at least about 95% or more, or 100% sequence identity with the nucleic acid sequence starting at base 10857 to base 16495 of SEQ ID NO: 94. The system comprises a nucleic acid construct comprising the donor polynucleotide, wherein the nucleic acid construct comprising the donor polynucleotide comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 2201 to base 2630 of SEQ ID NO: 94. The system also comprises an expression construct for expressing a gRNA targeting the soybean DD20 intergenic region, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence of SEQ ID NO: 103 or the nucleic acid sequence starting at base 2861 to base 3572 of SEQ ID NO: 94. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 103 or the nucleic acid sequence starting at base 2861 to base 3572 of SEQ ID NO: 94. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 94.

In some aspects, a system of the instant disclosure is a one-component system, wherein the Cas9 protein is linked to the Pong ORF2 protein, the donor construct is inserted in an expression construct expressing a GFP reporter, and the target nucleic acid locus is in a soybean DD20 intergenic region. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5490 to base 9399 of SEQ ID NO: 95. In some aspects, the nucleic acid expression construct for expressing a Pong ORF1 protein comprises at least about 75% or more, at least about 85% or more, at least about 95% or more, or 100% sequence identity with the nucleic acid sequence starting at base 5490 to base 9399 of SEQ ID NO: 95. The system also comprises a nucleic acid nucleic acid expression construct for expressing a Pong ORF2 protein linked to a Cas9 nuclease, wherein the expression construct for expressing the Pong ORF2 protein linked to a Cas9 nuclease comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 9582 to base 16938 of SEQ ID NO: 95. In some aspects, the expression construct for expressing the Pong ORF2 protein linked to a Cas9 nuclease comprises a nucleic acid sequence comprising at least about 75% or more, at least about 85% or more, at least about 95% or more, or 100% sequence identity with the nucleic acid sequence starting at base 9582 to base 16938 of SEQ ID NO: 95. The system comprises a nucleic acid construct comprising the donor polynucleotide, wherein the nucleic acid construct comprising the donor polynucleotide comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 4545 to base 2173 of SEQ ID NO: 95. The system also comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 4763 to base 5474 of SEQ ID NO: 95. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 95. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 95.

In some aspects, the system of the instant disclosure comprises a helper construct and a donor construct, wherein the helper construct comprises a nucleic acid expression construct for expressing Pong ORF1 and a nucleic acid expression construct for expressing Pong ORF2 protein linked to a Cas9 nuclease. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 981 to base 4890 of SEQ ID NO: 75. In some aspects, the nucleic acid expression construct for expressing a Pong ORF1 protein comprises at least about 75% or more, at least about 85% or more, at least about 95% or more, or 100% sequence identity with the nucleic acid sequence starting at base 981 to base 4890 of SEQ ID NO: 75. The system also comprises a nucleic acid expression construct for expressing a Pong ORF2 protein linked to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 12429 of SEQ ID NO: 75. In some aspects, the construct for expressing a Pong ORF2 protein linked to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 75. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 75. In some aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 75. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 75. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 75.

In some aspects, the donor polynucleotide is inserted in a nucleic acid expression construct encoding a GFP reporter, thereby inactivating the reporter. In some aspects, the expression construct is inserted in nucleic acid sequence in the genome of the cell. In some aspects, the target nucleic acid locus is in an Arabidopsis PDS3 gene.

In some aspects, the system of the instant disclosure comprises a helper construct and a donor construct. In some aspects, the donor construct comprises a nucleic acid expression construct encoding a GFP reporter. The donor nucleic acid construct is inserted into the expression construct thereby inactivating the reporter. In these aspects, the target nucleic acid locus is an Arabidopsis ADH1 gene. The helper construct comprises a nucleic acid expression construct for expressing Pong ORF1, a nucleic acid expression construct for expressing Pong ORF2 protein, and a nucleic acid construct for expressing a deCas9 nickase. The expression construct for expressing a Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 981 to base 4890 of SEQ ID NO: 89. In some aspects, the nucleic acid expression construct for expressing a Pong ORF1 protein comprises at least about 75% or more, at least about 85% or more, at least about 95% or more, or 100% sequence identity with the nucleic acid sequence starting at base 981 to base 4890 of SEQ ID NO: 89. The system also comprises a nucleic acid expression construct for expressing a Pong ORF2 protein, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 8215 of SEQ ID NO: 89. In some aspects, the construct for expressing a Pong ORF2 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89. The system also comprises a nucleic acid expression construct for expressing a deCas9 nickase, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 8218 to nucleotide 13856 of SEQ ID NO: 89. In some aspects, the construct for expressing a deCas9 nickase protein comprises a nucleic acid sequence comprising at least about 75% or more, at least about 85% or more, at least about 95% or more, or 100% sequence identity with the nucleic acid sequence starting at nucleotide 8218 to nucleotide 13856 of SEQ ID NO: 89. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 89. In some aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89. In some aspects, the helper construct is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 89. In some aspects, the helper construct is encoded on a plasmid comprising a nucleic acid sequence comprising at least about 75% or more, at least about 85% or more, at least about 95% or more, or 100% sequence identity with the nucleic acid sequence of SEQ ID NO: 89.

In some aspects, the system of the instant disclosure comprises a helper construct and a donor construct. In some aspects, the donor construct comprises a nucleic acid expression construct encoding a GFP reporter, wherein the donor nucleic acid construct is inserted into the expression construct thereby inactivating the reporter. In these aspects, the target nucleic acid locus is an Arabidopsis ACT8 gene. The helper construct comprises a nucleic acid expression construct for expressing Pong ORF1 and a nucleic acid expression construct for expressing Pong ORF2 protein linked to a Cas9 nuclease. The expression construct for expressing a Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 981 to base 4890 of SEQ ID NO: 91. In some aspects, the nucleic acid expression construct for expressing a Pong ORF1 protein comprises at least about 75% or more, at least about 85% or more, at least about 95% or more, or 100% sequence identity with the nucleic acid sequence starting at base 981 to base 4890 of SEQ ID NO: 91. The system also comprises a nucleic acid expression construct for expressing a Pong ORF2 protein linked to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 12429 of SEQ ID NO: 91. In some aspects, the construct for expressing a Pong ORF2 protein linked to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 91. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 91. In some aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 91. In some aspects, the helper construct is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 91. In some aspects, the helper construct is encoded on a plasmid comprising a nucleic acid sequence comprising at least about 75% or more, at least about 85% or more, at least about 95% or more, or 100% sequence identity with the nucleic acid sequence of SEQ ID NO: 91.

The donor construct comprises a nucleic acid expression construct comprising a promoter operably linked to a polynucleotide sequence encoding GFP, wherein the donor polynucleotide inserted in the nucleic acid expression construct. In some aspects, the GFP expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 3037 clockwise to base 665 of SEQ ID NO: 90. In some aspects, the GFP expression construct comprises a nucleic acid sequence comprising at least about 75% or more, at least about 85% or more, at least about 95% or more, or 100% sequence identity with the nucleic acid sequence starting at base 3037 clockwise to base 665 of SEQ ID NO: 90. In some aspects, the donor construct is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 90. In some aspects, the donor construct is encoded on a plasmid comprising a nucleic acid sequence comprising at least about 75% or more, at least about 85% or more, at least about 95% or more, or 100% sequence identity with the nucleic acid sequence of SEQ ID NO: 90.

In some aspects, the programmable targeting system of the instant disclosure comprises a CRISPR nuclease system comprising dCas9 and a gRNA. In some aspects, the dCas9 nuclease is linked to Pong ORF2 by one copy of a G4S linker of SEQ ID NO: 64. In some aspects, the Pong ORF2 protein linked to the dCas9 nuclease by one copy of a G4S linker of SEQ ID NO: 64 comprises an amino acid sequence encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 110. In some aspects, the Pong ORF2 protein linked to the dCas9 nuclease by one copy of a G4S linker of SEQ ID NO: 64 comprises an amino acid sequence encoded by a nucleic acid sequence comprising at least about 75% or more, at least about 85% or more, at least about 95% or more, or 100% sequence identity with a nucleic acid sequence of SEQ ID NO: 110.

In some aspects, the Pong ORF2 protein linked to the dCas9 nuclease by one copy of a G4S linker of SEQ ID NO: 64 is expressed using an expression construct for expressing the Pong ORF2 protein linked to the dCas9 nuclease by one copy of a G4S linker of SEQ ID NO: 64, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 115. In some aspects, the expression construct comprises a nucleic acid sequence comprising at least about 75% or more, at least about 85% or more, at least about 95% or more, or 100% sequence identity with SEQ ID NO: 115. In some aspects, the genetically modified cell is an Arabidopsis thaliana cell.

In some aspects, the Pong ORF2 protein linked to the Cas9 nuclease by three copies of a G4S linker of SEQ ID NO: 64 is expressed using an expression construct for expressing the Pong ORF2 protein linked to the Cas9 nuclease by three copies of a G4S linker of SEQ ID NO: 64, wherein the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 104. In some aspects, the expression construct comprises a nucleic acid sequence comprising at least about 75% or more, at least about 85% or more, at least about 95% or more, or 100% sequence identity with SEQ ID NO: 104. In some aspects, the genetically modified cell is a soybean cell.

### III. Cells

Another aspect of the instant disclosure encompasses a cell, a tissue, or an organism comprising an engineered system described in Section I above. One or more components of the engineered system in the cell may be encoded by one or more nucleic acid constructs of a system of nucleic acid constructs as described in Section II above.

A variety of cells are suitable for use in the methods disclosed herein. The cell may be a prokaryotic cell. Alternatively, the cell is a eukaryotic cell. For example, the cell may be a prokaryotic cell, a human mammalian cell, a non-human mammalian cell, a non-mammalian vertebrate cell, an invertebrate cell, an insect cell, a plant cell, a yeast cell, or a single cell eukaryotic organism. The cell may also be a one-cell embryo. For example, a non-human mammalian embryo including rat, hamster, rodent, rabbit, feline, canine, ovine, porcine, bovine, equine, plant, and primate embryos. The cell may also be a stem cell such as embryonic stem cells, ES-like stem cells, fetal stem cells, adult stem cells, and the like. The cell may be in vitro, ex vivo, or in vivo (i.e., within an organism or within a tissue of an organism).

Non-limiting examples of suitable mammalian cells or cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells; baby hamster kidney (BHK) cells; mouse myeloma NSO cells; mouse embryonic fibroblast 3T3 cells (NIH3T3); mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells; mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9 L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; Afrimay green monkey kidney (VERO-76) cells. An extensive list of mammalian cell lines may be found in the Amerimay Type Culture Collection catalog (ATCC, Manassas, VA).

The cell may be a plant cell, a plant part, or a plant. Plant cells include germ cells and somatic cells. Non-limiting examples of plant cells include parenchyma cells, sclerenchyma cells, collenchyma cells, xylem cells, and phloem cells. Plant parts include, but are not limited to, stems, roots, ovules, stamens, leaves, embryos, meristematic regions, callus tissue, gametophytes, sporophytes, pollen, microspores, and the like. The plant can be a monocot plant or a dicot plant. For instance, the plant can be soybean; maize; sugar cane; beet; tobacco; wheat; barley; poppy; rape; sunflower; alfalfa; sorghum; rose; carnation; gerbera; carrot; tomato; lettuce; chicory; pepper; melon; cabbage; oat; rye; cotton; millet; flax; potato; pine; walnut; citrus (including oranges, grapefruit etc.); hemp; oak; rice; petunia; orchids; Arabidopsis; broccoli; cauliflower; brussels sprouts; onion; garlic; leek; squash; pumpkin; celery; pea; bean (including various legumes); strawberries; grapes; apples; cherries; pears; peaches; banana; palm; cocoa; cucumber; pineapple; apricot; plum; sugar beet; lawn grasses; maple; teosinte; Tripsacum; Coix; triticale; safflower; peanut; cassava, and olive.

The invention also provides an agricultural product produced by any of the described transgenic plants, plant parts, and plant seeds. Agricultural products include, but are not limited to, plant extracts, proteins, amino acids, carbohydrates, fats, oils, polymers, vitamins, and the like.

### IV. Methods

A further aspect of the present disclosure encompasses a method of targeted insertion of nucleic acid sequence into a target nucleic acid locus in a cell. In a method of the instant disclosure, the cell can be ex vivo or in vivo. The locus can be in a chromosomal DNA, organellar DNA, or extrachromosomal DNA. The method can be used to insert a single donor polynucleotide or more than one donor polynucleotide at one or more target loci.

The method comprises providing or having provided an engineered system for generating a genetically modified cell and introducing the system into the cell. The method further comprises maintaining the cell under appropriate conditions such that the donor polynucleotide is inserted in the target locus. Optionally, the method further comprises identifying an accurate insertion of the donor polynucleotide in the nucleic acid locus. The engineered system can be as described in Section I; nucleic acid constructs encoding one or more components of the homologous recombination compositions can be as described in Section II; and the cells can be as described in Section III.

Insertion of the donor polynucleotide into a target nucleic acid locus in a cell can have a number of uses known to individuals of skill in the art. For instance, insertion of the donor polynucleotide can introduce cargo nucleic acid sequences of interest into nucleic acid sequences in a cell, including genes of interest or regulatory nucleic acid sequences of interest. Alternatively, insertion of a donor polynucleotide can be used to introduce nucleic acid modifications in nucleic acid sequences in the cell. The system can be used to modulate transcriptional or post-transcriptional expression of an endogenous nucleic acid sequence in the cell, to investigate RNA-protein interactions, or to determine the function of a protein or RNA, or investigate RNA-protein interactions, or to alter the stability, accumulation, and protein production from the RNA.

In general, cargo nucleic acid sequences can be introduced into a nucleic acid sequence of a cell by flanking the nucleic acid sequence to be introduced with the transposition sequences compatible with the transposase. Introduced cargo nucleic acid sequences can include, without limitation, nucleic acid sequences encoding herbicide resistance, disease resistance such as viral coat proteins and R gene families, insect resistance such as Bt toxin genes, antibiotic resistance, short RNAs, reporters, programmable nucleic acid-modification systems, epigenetic modification systems, regulatory elements, viral vectors, agronomic traits of interest such drought and salinity resistance, and any combination thereof. Non-limiting examples of cargo nucleic acid sequences include Bt toxin tenes (Cry Genes), RNAi (RNA Interference) constructs, pathogen-derived resistance genes, R gene families, herbicide resistance genes, nitrogen fixation genes (Nodulation Genes), drought tolerance tenes, salinity tolerance genes, cold tolerance genes, vitamin and nutrient enrichment genes, fruit ripening control genes, photosynthetic efficiency genes, flower color modification genes, plant growth regulator genes, phytoremediation genes, altered oil or protein content genes, biofortification genes, and aroma and flavor enhancement genes.

In some aspects, a method of the instant disclosure comprises altering expression of a gene of interest. The method comprises introducing expression regulatory elements to a location on the genome where expression of a gene of interest is controlled. In some aspects, the regulatory elements are heat shock enhancer elements. In some aspects, the method comprises introducing an array of six heat-shock enhancer elements flanked by the mPing transposition sequences for insertion into the promoter of the Arabidopsis ACT8 gene. These enhancers have a short size and regulate expression of the gene irrespective of the orientation of the introduced sequences. Donor constructs comprising heat-shock enhancer elements flanked by the mPing transposition sequences can be as described in Sections I(b) and Section II.

In some aspects, a method of the instant disclosure is used to introduce a herbicide resistance gene. Non-limiting examples of genes that can be used in cargo nucleic acids of the instant disclosure to introduce herbicide resistance include EPSPS (5-Enolpyruvylshikimate-3-Phosphate Synthase) that can provide resistance to glyphosate herbicides, such as Roundup, PAT (Phosphinothricin Acetyltransferase) that can confer resistance to glufosinate herbicides, including Liberty and Basta, modified ALS (Acetolactate Synthase) genes that can confer resistance to sulfonylurea and imidazolinone herbicides, BAR (Bialaphos Resistance) that can provide resistance to herbicides like Bialaphos and phosphinothricin (the active ingredient in glufosinate herbicides), modified ACCase (Acetyl-CoA Carboxylase) genes that can provide resistance to ACCase-inhibiting herbicides, such as clethodim and sethoxydim, modified PPO (Protoporphyrinogen Oxidase) genes that can provide resistance to saflufenacil, GST (Glutathione S-Transferase) genes that can be used to enhance the plant's ability to detoxify a range of herbicides by conjugating them with glutathione, rendering them less toxic, Vip3A (Vegetative Insecticidal Protein) gene that can confer resistance to some herbivorous insects that damage crops alongside herbicide resistance, modified HPPD (4-Hydroxyphenylpyruvate Dioxygenase) genes that can confer resistance to certain herbicides, like mesotrione, inhibit the HPPD enzyme, AAD-12 (Aryloxyalkanoate Dioxygenase-12) gene that can provide resistance to 2,4-D herbicides, and DSF (Dinitroaniline Herbicide Resistance). In some aspects, a method of the instant disclosure comprises introducing resistance to bialophos herbicide. In some aspects, a method of the instant disclosure comprises introducing a donor construct comprising an expression construct expressing the BAR gene flanked by the mPing transposition sequences into a cell. Donor constructs comprising heat-shock enhancer elements flanked by the mPing transposition sequences can be as described in Sections I(b) and Section II.

(a) Introduction into the Cell

The method comprises introducing the engineered system into a cell of interest. The engineered system may be introduced into the cell as a purified isolated composition, purified isolated components of a composition, as one or more nucleic acid constructs encoding the engineered system, or combinations thereof. Further, components of the engineered system can be separately introduced into a cell. For example, a transposase, a donor polynucleotide, and a programmable targeting nuclease can be introduced into a cell sequentially or simultaneously.

The engineered system described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, implantable devices, magnetofection, lipofection, impalefection, optical transfection, proprietary agent-enhanced uptake of nucleic acids, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (b) Culturing a Cell

The method further comprises maintaining the cell under appropriate conditions such that the donor polynucleotide is inserted in the target locus. When the cell is in tissue ex vivo, or in vivo within an organism or within a tissue of an organism, the tissue and/or organism may also be maintained under appropriate conditions for insertion of the donor polynucleotide. In general, the cell is maintained under conditions appropriate for cell growth and/or maintenance. Those of skill in the art appreciate that methods for culturing cells are known in the art and may and will vary depending on the cell type. Routine optimization may be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306; Taylor et al., (2012) Tropical Plant Biology 5:127-139.

In some aspects, the method further comprises identifying an accurate insertion of the donor polynucleotide using methods known in the art. Upon confirmation that an accurate insertion has occurred, single cell clones may be isolated. Additionally, cells comprising one accurate insertion may undergo one or more additional rounds of targeted insertions of additional polynucleotides.

### V. Kits

A further aspect of the present disclosure encompasses kits for generating a genetically modified cell. The kit comprises one or more engineered systems detailed above in Section I. The engineered systems can be encoded by a system of one or more nucleic acid constructs encoding the components of the system as described above described above in Section II. Alternatively, the kit may comprise one or more cells comprising one or more engineered systems, one or more nucleic acid constructs, or combinations thereof.

A further aspect of the present disclosure provides a system of one or more nucleic acid constructs encoding the components of the system described above

The kits may further comprise transfection reagents, cell growth media, selection media, in-vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), an internet address that provides the instructions, and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, the term “compatible transposition sequences” refers to any transposition sequences recognized by the transposase for transposition. For instance, the transposition sequences can be transposition sequences of the TE from which the transposase is derived, or from another autonomous or non-autonomous TE recognized by the transposase for transposition.

As used herein, the term “engineered” when applied to a targeting protein refers to targeting proteins modified to specifically recognize and bind to a nucleic acid sequence at or near a target nucleic acid locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The term “nucleic acid modification” refers to processes by which a specific nucleic acid sequence in a polynucleotide is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

As used herein, the term “encode” is understood to have its plain and ordinary meaning as used in the biological fields, i.e., specifying a biological sequence. For instance, when a construct is encoding a protein of the system, the term is understood to mean that the construct further comprises nucleic acid sequences required for expressing the components of the system.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Targeted Integration of a Transposable Element

Transgenesis in plants is accomplished via bombardment or agrobacterium-mediated transformation and results in the integration of foreign DNA into a plant's genome. During this process, the transgene integration site within the plant DNA is not controlled, and follow-up experiments must be performed to determine where in the genome the transgene integrated. En mass transformation experiments have demonstrated that the integration typically occurs at sites of open chromatin configuration, such as actively transcribing genes, however integration into heterochromatic closed chromatin can also occur. Transgene integration into or near genes can generate new mutations or alter the regulation of nearby genes, while insertions into heterochromatic regions are often not permissive to the desired high levels of transgene expression or do not provide stable expression over multiple generations. Insertion of transgenes is also associated with mutations (deletions and rearrangements) of the target region and transferred DNA. In addition, to study or create a product from a gene of interest, it needs to be taken out of its native context and added back to the plant as a transgene, and key distal regulatory enhancers or repressor elements can be missed or rearranged during this process. The lack of user-defined control of transgene integration site generates variability and inconsistency in experiments and products.

The control of transgene integration site is desired to direct transgenes to the same expression-permissive regions of the genome (to reduce variability), to add sequences to genes at their native locations, and/or to maintain gene order on the chromosome. Multiple attempts have been made to overcome these issues and perform target site-directed integration. The FLP-FRT recombination system has been used to reproducibly target transgene insertion into one location in plant genomes. However, this insertion site must also be transgenic to carry the correct targeting sequences. Current methods to insert DNA into any user-defined targeted region of a plant genome involve homology-directed repair (HDR) off a provided DNA template after a double-strand DNA break induced by a Meganuclease, Zinc Finger Nuclease, TALEN or CRISPR/Cas9 (or related) system. In plants, currently available tools using targeted insertion of a transgene via HDR are inefficient for two reasons. First, the complementary repair template and nuclease system must be added to the cell via traditional transgenesis, which particularly in crop plants is laborious. Second, plant cells favor the resolution of double-strand DNA breaks by the non-homology end joining (NHEJ) pathway, which bypasses the integration of new DNA.

Recently, research has uncovered naturally-occurring fusions between transposase proteins and the CRISPR/Cas system in prokaryotes. The CRISPR/Cas system provides sequence specificity to the transposase for selection of the integration site, and was proven to be programmable by altering the sequence of the CRISPR guide RNA (gRNA). However, none of the systems currently available that use CRISPR-targeting of a transposase protein were successful in targeting to a specific gene location in eukaryotic cells. To date, the programmability of transposase-mediated integration of DNA has not been accomplished in a eukaryote.

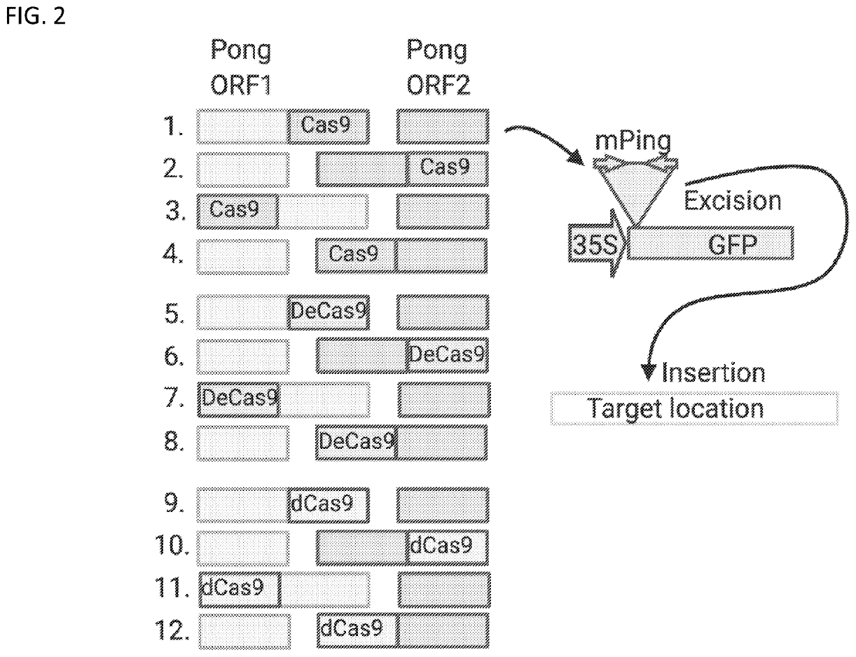

In an attempt to overcome the difficulties in accomplishing insertion of a transgene into a target locus, the inventors linked a TE-encoded transposase protein to the CRISPR/Cas9 system to achieve targeted integration of DNA in plants. The inventors reasoned that the transposase protein would need to have two features to broadly function in this system. First, a wide host-range of functionality in plants was desired to create a universal tool for plant biology. Second, using split-transposase proteins (where the single transposase was encoded by two proteins that function together to achieve excision and insertion) would have a lower probability of disturbing protein function. It was reasoned that the rice mPing/Pong system would provide the highest probably of functioning when linked to Cas9, as the Pong transposase is split into two proteins (ORF1 and ORF2) and can mobilize the mPing non-autonomous (non-protein coding) TE in a range of plant species. An mPing/Pong engineered system was used that had the Pong transposase ORF1 and ORF2 immobilized by the removal of the Pong TIRs. In this system, mPing excision can be visualized by its removal from a constitutively expressed GFP gene (FIG. 1). The Pong ORF1/ORF2 system was engineered with the G4S (GSSSS) flexible protein linker to allow efficient fusions to Cas9 proteins on either the N- or C-terminus of ORF1 or ORF2, and an SV40 nuclear localization signal (NLS) was added to these protein fusions. Three versions of the Cas9 protein were used, the catalytically active Cas9, the single-stranded nickase deCas9, and the catalytically inactive dCas9. A total of 12 constructs were generated (3 Cas9 proteins×4 ORF1/ORF2 positions; FIG. 2) with a gRNA known to target the Arabidopsis PDS3 gene.

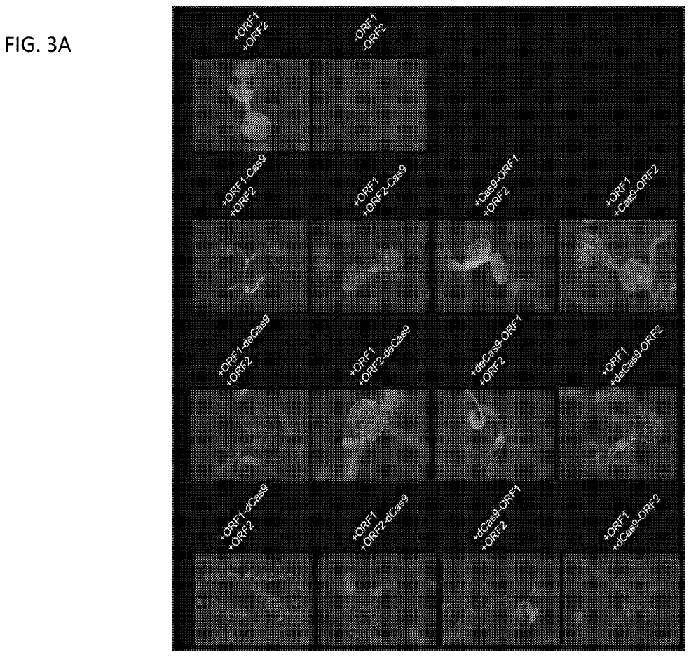

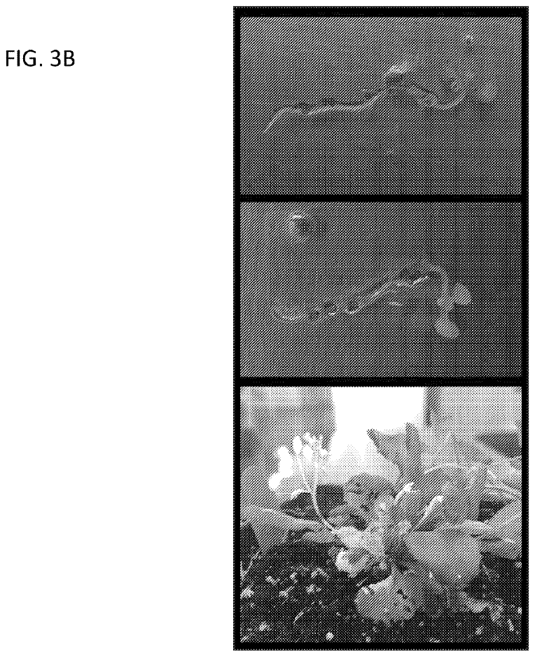

To determine if the Pong transposase was functional when linked to Cas9 derivatives, GFP fluorescence was visualized in seedlings. GFP fluorescence is a marker of mPing excision from the GFP donor site, and this fluorescence was detected for all 12 fusion proteins, but not the negative control without ORF1/ORF2 (FIG. 3A), verifying that ORF1 and ORF2 are co-creating a functional transposase protein even while linked to Cas9. A functional CRISPR/Cas9 system was verified through the observation of white seedlings and sectors in plants with the Cas9 and deCas9 proteins (in this experiment, dCas9 plants did not display white plants or sectors) (FIG. 3B). Overall, the results demonstrate that fusion of the Cas9 and transposase proteins does not stop their function.

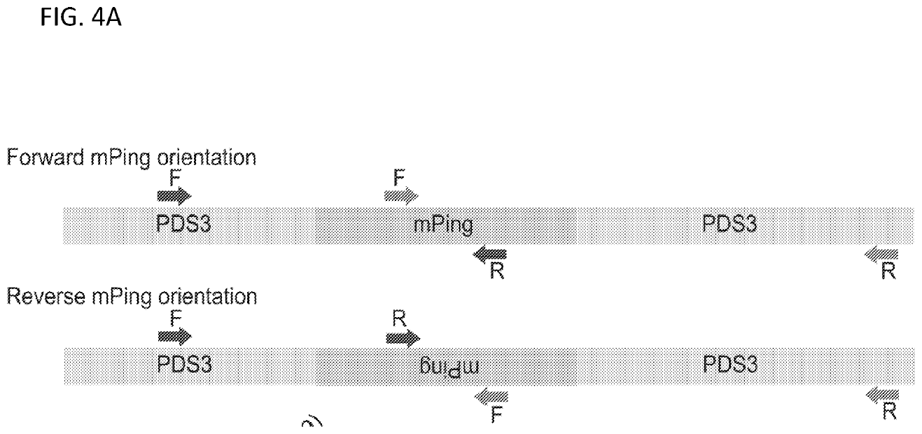

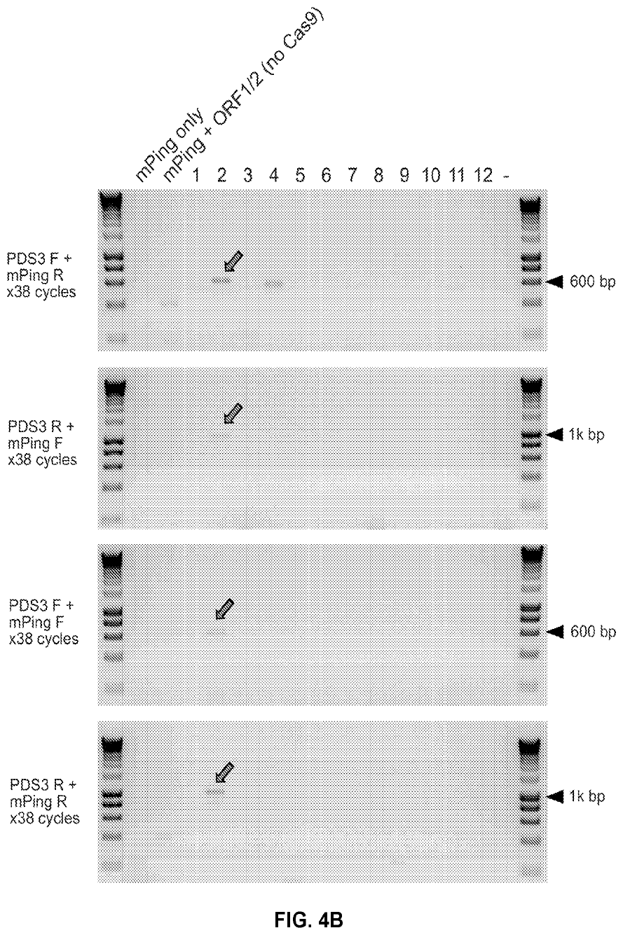

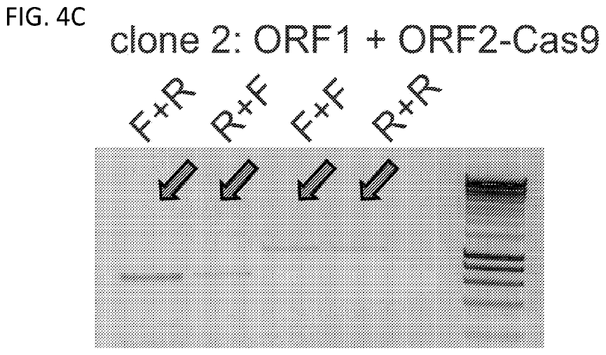

A PCR amplification strategy was used to detect targeted mPing insertions into the Arabidopsis PDS3 gene (FIG. 4A). T2 seedling pools were screened using negative control lines that either lack ORF1/ORF2, or that lack the Cas9 fusion (FIG. 4B). It was found that clone #2 displayed the correct size PCR band in all PCR assays (FIG. 4B). The PCR can identify mPing insertions in the forward or reverse orientation (FIG. 4A), and the fact that clone #2 amplified for both suggests that there is more than one mPing insertion in this pool of plants. Clone #2 encodes for ORF1+ORF2-Cas9, where ORF2 has a C-terminal fusion to the Cas9 protein. This data demonstrates targeted insertion of mPing into the PDS3 gene using a targeting nuclease having full double stranded cleavage activity of Cas9.

### Example 2. Characterization of Target Site Insertions

The target-site PCR assay was replicated (FIG. 4C), and PCR products cloned and sequenced. In all, 36 clones were sequenced. The sequenced clones represent at least nine (9) unique targeted transposition events (FIG. 5). Both mPing forward and reverse orientation insertions were identified, demonstrating the random directionality of the targeted insertion event.

The targeted insertion occurred between the third and fourth base of the gRNA target sequence, as expected based on the known cleavage activity of Cas9 (FIG. 5). The results show that mPing is intact in each sequenced clone except one. In each case there is one target site duplication, on either the 5′ or 3′ of mPing. Additional single-base insertions are found in some clones. The sequencing represents at least nine distinct events, meaning that mPing inserted into the PDS3 gene in the line with clone #2 at least nine different times. Most insertions have either intact or partial TTA/TAA sequence on only one end of the insertion. This sequence originates from the donor site and is part of the known target site duplication (TSD) of the Pong/mPing TE system. The presence of only one TSD, rather than one on either side of the TE insertion, signifies that Cas9 created a blunt cut at the insertion site, but the transposase protein made a staggered cut at the donor site before the integration event. This demonstrates that both the Cas9 and transposase proteins are functional for generating this set of insertions.

For each insertion, the gRNA target sequence was preserved and mPing had inserted at the expected Cas9 cleavage point between the third and fourth nucleotide. In all but one sequence read the mPing element is complete, with only single base insertions. The lack of deletions or other insertions at these insertion sites demonstrates the seamless repair of the insertion events by the transposase protein compared to typical sites of blunt-end DNA breaks.

### Example 3. Integration into any DNA Break

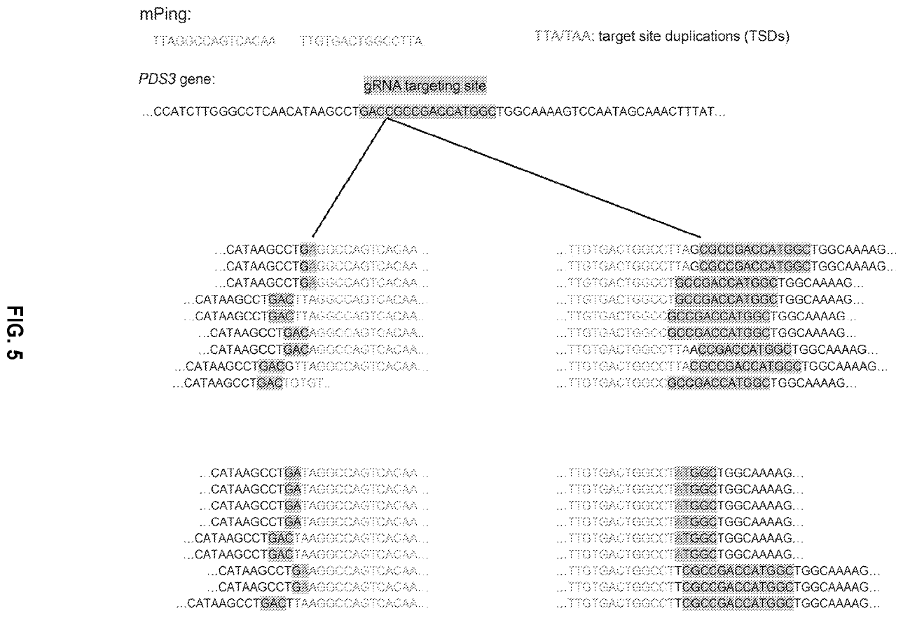

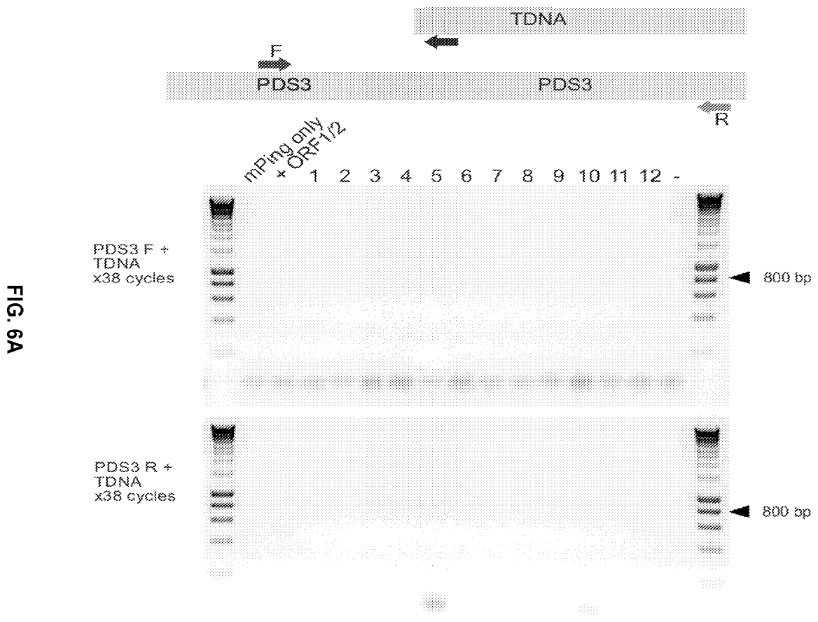

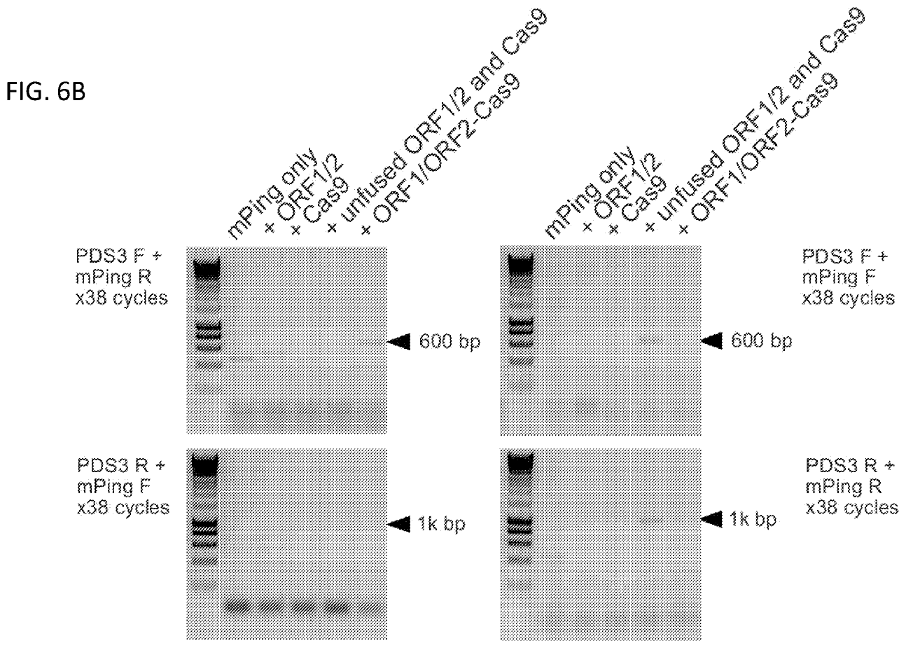

Several previous reports have demonstrated that transgenes will insert at a low frequency into any site of double-strand break. To determine if the mPing targeted insertion detected in Examples 1 and 2 requires the transposase protein, a PCR assay was performed for the integration of the transgene backbone encoding the ORF2-Cas9 protein into the DNA break generated at PDS3. It was reasoned that if the mPing insertion into PDS3 was a product of transgene insertion, rather than transposition, it would be equally likely to detect other parts of the transgene at this insertion site location. However, transgene was detected at PDS3 (FIG. 6A), demonstrating that mPing insertion requires the transposase to excise the mPing element from the donor position.

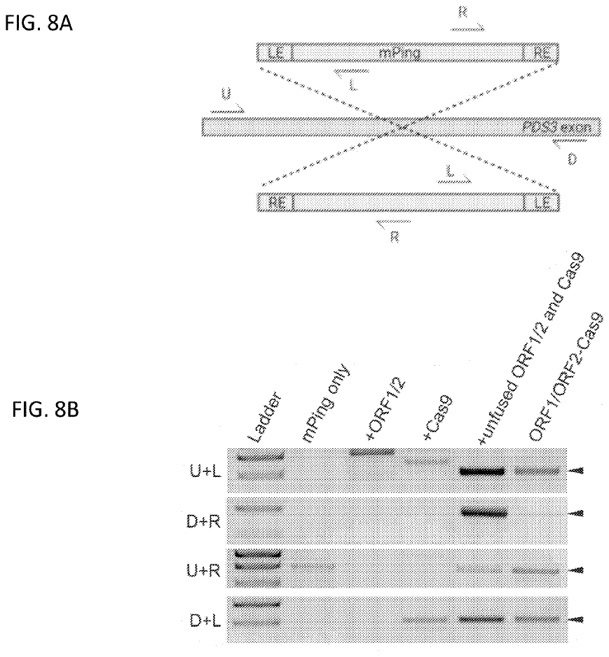

Next, it was assayed whether it was essential that the transposase protein and Cas9 were directly linked, or if both proteins unlinked in the same cell could perform targeted insertion. It was discovered that in some cases, the two proteins could be unlinked and targeted insertion would take place (FIG. 6B). At the same time, it was demonstrated that both proteins are functional and that in this instance, the catalytic activity of Cas9 is used (FIG. 6B). Together, this data demonstrates that to obtain targeted insertion, it is essential that the transposase excise the element out of the donor position, and that Cas9 cleave the insertion site, but the two proteins do not necessarily need to be linked together (see FIGS. 8A and 8B and Example 5).

### Example 4. Programmability of Target Sites

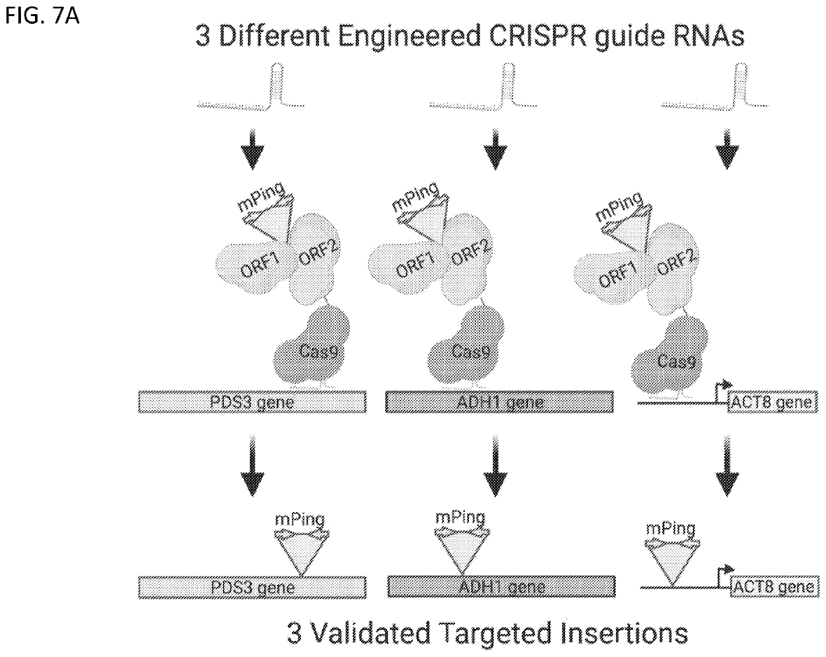

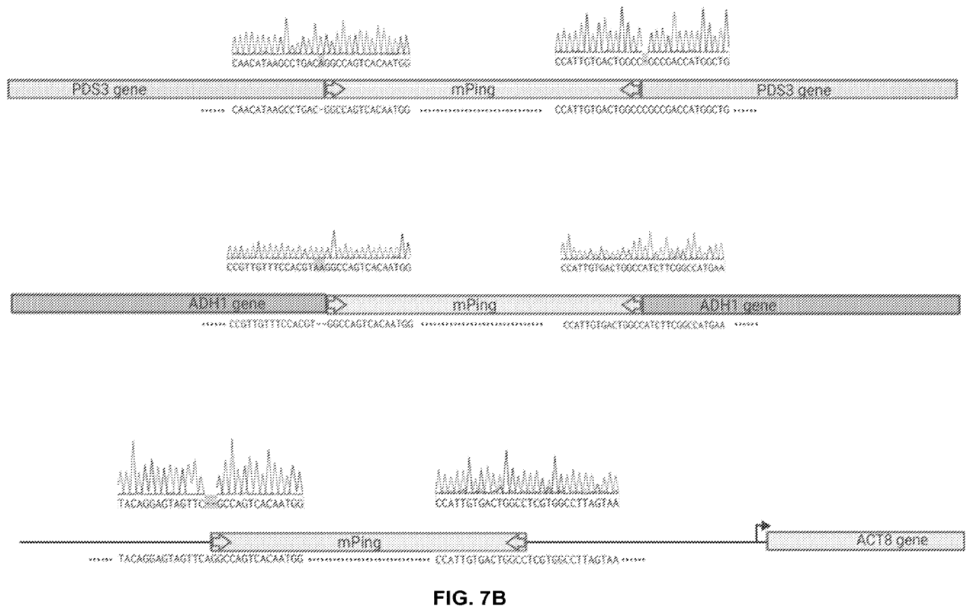

Multiple sites in the Arabidopsis genome were targeted using the system of the instant disclosure. Two additional gRNAs were designed for integration into two additional target loci; the ADH1 gene and a non-coding region upstream of the ACT8 gene of Arabidopsis. The gRNAs were used in a system described herein to integrate mPing into the two target loci (FIG. 7A). FIG. 7B shows the Sanger sequencing results of junctions of each identified target insertion into the PDS3 gene, the ADH1 gene, and the promoter of ACT8 gene. The chromatograms above the sequence show the sequences at the insertion sites. The sequences below mPing are the expected sequence if a perfect “seamless” insertion is obtained. These results clearly confirm that the insertion of a donor polynucleotide is surprisingly and unexpectedly inserted on target and unexpectedly accurate and seamless.

### Example 5. Direct Fusion of the Transposase Proteins ORF1 and ORF2 to the Nuclease is not Required for Targeted Insertions

Using methods described in Example 3, whether a system wherein the transposase proteins ORF1 and ORF2 are not directly linked to the Cas9 nuclease was tested. FIG. 8A shows that mPing can be targeted to the Arabidopsis PDS3 gene by the CRISPR gRNA and can insert in either the forward direction (above the PDS3 region) or reverse direction (below the PDS3 region). A combination of 2 out of 4 PCR primers corresponding to the PDS3 exon (U,D) and the mPing gene (R, L) were used. FIG. 8A shows the location of these 4 PCR primers (R,L,U,D) for orientation.

The mPing targeted insertion was detected with PCR using the primer sets from part A. FIG. 8B shows a representative agarose gel with PCR products observed. Arrowheads denote the correct size of the PCR products for each set of primers. “mPing only”, “+ORF1/2” and “+Cas9” are negative controls. Any bands from these lanes near the correct size were sequenced and shown not to be specific targeted insertions of mPing. The bands shown in the “+unlinked ORF1/2 and Cas9” lane show that using unlinked constructs can generate real targeted insertions, as does the biological replicate of ORF2 linked to Cas9 in the “ORF1/ORF2-Cas9” lane. All PCR products from this assay were also verified by Sanger sequencing. These data confirm the results from FIG. 6B and demonstrate that direct fusion of the transposase proteins to the nuclease is not required for targeted insertions.

### Example 6: Targeted Insertion Driven by Single Transgene Vector

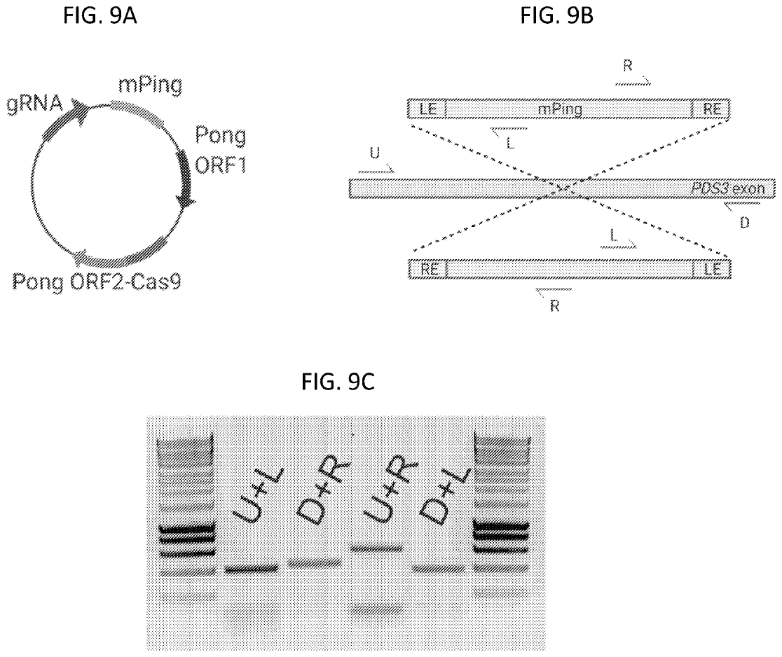

In the previously described experiments, the system comprised a donor construct and a helper construct. Here, a single transgene vector was developed containing all the elements required for targeted insertion in a plant cell. The vector is diagrammed in FIG. 9A and contains the CRISPR/Cas9 system (including gRNA), the mPing donor element, and ORF1 and ORF2 transposase proteins.

Using methods described in the examples above, mPing was targeted to the Arabidopsis PDS3 gene by the CRISPR gRNA. As shown in FIG. 9B, mPing can insert in either the forward direction (above the PDS3 region) or reverse direction (below the PSD3 region). The location of 4 PCR primers (R, L, U, D) are shown for orientation. FIG. 9C shows a representative agarose gel with PCR detection of mPing targeted insertion in the Arabidopsis genome using the primer sets from part B. The largest PCR fragment for each primer set is the correct size and was Sanger sequenced to ensure that it is a bonafide targeted insertion of mPing into the PDS3 gene.

### Example 7: Targeted and Seamless Integration in Plant Genomes Using CRISPR-Transposases

### Introduction

Transgenesis in plants is accomplished via bombardment or agrobacterium-mediated transformation and results in the integration of foreign DNA into a plant's genome. During this process, the transgene integration site within the plant DNA is not controlled, and follow-up experiments must be performed to determine where in the genome the transgene integrated. En mass transformation experiments have demonstrated that the integration typically occurs at sites of open chromatin configuration, such as actively transcribing genes, however integration into heterochromatic closed chromatin can also occur. Transgene integration into or near genes can generate new mutations or alter the regulation of nearby genes, while insertions into heterochromatic regions are often not permissive to the desired high levels of transgene expression or do not provide stable expression over multiple generations. Insertion of transgenes is also associated with mutations (deletions and rearrangements) of the target region and transferred DNA. In addition, to study or create a product from a gene of interest, it needs to be taken out of its native context and added back to the plant as a transgene, and key distal regulatory enhancers or repressor elements can be missed or rearranged during this process. The lack of user-defined control of transgene integration site generates variability and inconsistency in experiments and products.

The control of transgene integration site is desired to direct transgenes to the same expression-permissive regions of the genome (to reduce variability), to add sequences to genes at their native locations, and/or to maintain gene order on the chromosome. Multiple attempts have been made to overcome these issues and perform targeted site-directed integration. Recombination systems have been used to reproducibly target transgene insertion into one location in plant genomes, however, this insertion site must also be transgenic to carry the correct targeting sequences. Current methods to insert DNA into any user-defined targeted region of a plant genome involve homology-directed repair (HDR) off a provided DNA template after a double-strand DNA break induced by a Meganuclease, Zinc Finger Nuclease, TALEN or CRISPR/Cas9 (or related) system. In plants, targeting insertion of a transgene via HDR is inefficient for two reasons. First, the complementary repair template and nuclease system must be added to the cell via traditional transgenesis, which particularly in crop plants is laborious. Second, plant cells favor the resolution of double-strand DNA breaks by the non-homology end joining (NHEJ) pathway, which bypasses the integration of new DNA. Therefore, addition of custom sequences to a targeted location in a plant genome is laborious, requiring screening for a low-frequency event. In addition, because free ends of DNA are exposed during this process, the ends of the inserted fragment of DNA or the native DNA at the insertion site is often subject to degradation, creating deletions and unintended base changes at the HDR site.

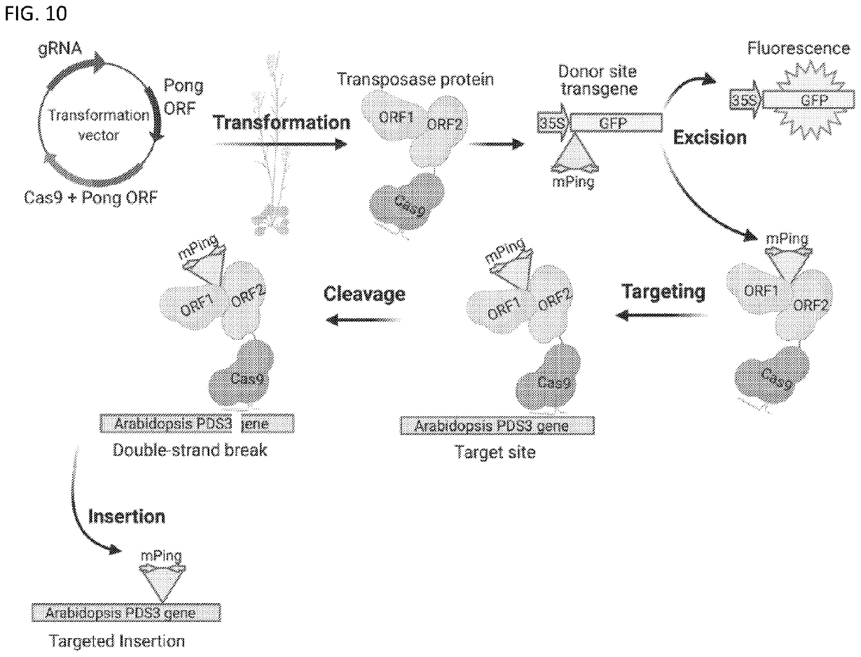

Transposases are transposable element (TE)-derived proteins that naturally mobilize pieces of DNA from one location in the genome to another. Transposases function by binding the repeated ends of a TE called the terminal inverted repeats (TIRs) within the same TE family. The transposase cleaves the DNA, removing the TE from the excision/donor site, then cleaves and integrates the TE at the insertion site. Plant transposases select their insertion site by chromatin context and DNA accessibility but are not targeted to individual regions or specific sequences of plant genomes. Recently, research has uncovered naturally-occurring fusions between transposase proteins and the CRISPR/Cas system in prokaryotes. The CRISPR/Cas system provides sequence specificity to the transposase for selection of the integration site, and was proven to be programmable by altering the sequence of the CRISPR guide RNA (gRNA). Several laboratories have taken the approach to identify natural Cas protein fusions to transposable elements in prokaryotic genomes, with the intent of moving these fusion proteins into eukaryotes. In human cell culture, CRISPR-targeting of a transposase protein has been attempted but failed to target to a specific gene location, although the integration into targeted repetitive retrotransposon sites were enriched. The inventors took the approach of starting with a transposase protein known to work in a wide variety of plants, and Cas9 and CFP1, which have also been shown to work in plants. Rather than identifying a natural fusion in a prokaryotic genome, both of these proteins were artificially used at the same time, including fusing these proteins together, to accomplish targeted insertion in a plant genome. An overview of this process is shown in FIG. 10.

### Results

### Targeted Integration of a Transposable Element

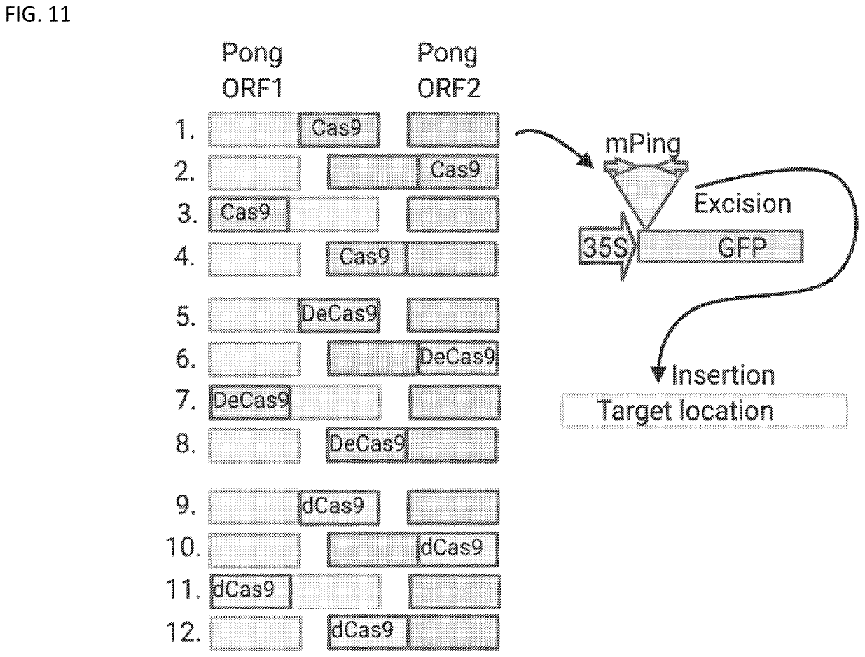

The goal was to fuse a TE-encoded transposase protein to the CRISPR/Cas9 system to achieve targeted integration of DNA in plants. The reason lies in that the transposase protein would need to have two features to broadly function in this system. First, a wide host-range of functionality in plants was desired to create a universal tool for plant biology. Second, using split-transposase proteins (where the single transposase was encoded by two proteins that function together to achieve excision and insertion) would have a lower probability of disturbing protein function. It was reasoned that the rice mPing/Pong system would provide the highest probably of functioning when linked to Cas9, as the Pong transposase is split into two proteins (ORF1 and ORF2) and can mobilize the mPing non-autonomous (non-protein coding) TE in a range of plant species. mPing/Pong engineered system was obtained where the Pong transposase ORF1 and ORF2 were immobilized by the removal of the Pong TIRs, and mPing excision can be visualized by its removal from a constitutively expressed GFP gene (cartoons in FIG. 11). The Pong ORF1/ORF2 system was engineered with the G4S (GSSSS; SEQ ID NO: 64) flexible protein linker to allow efficient fusions to Cas9 proteins on either the N- or C-terminus of ORF1 or ORF2 and added an SV40 nuclear localization signal (NLS) to these protein fusions. Three versions of the Cas9 protein where used, the catalytically active Cas9, the single-stranded nickase deCas9, and the catalytically inactive dCas9. A total of 12 constructs were generated (3 Cas9 proteins×4 ORF1/ORF2 positions) (FIG. 11) with a gRNA known to target the Arabidopsis PDS3 gene (https://doi.org/10.1038/nbt.2655).

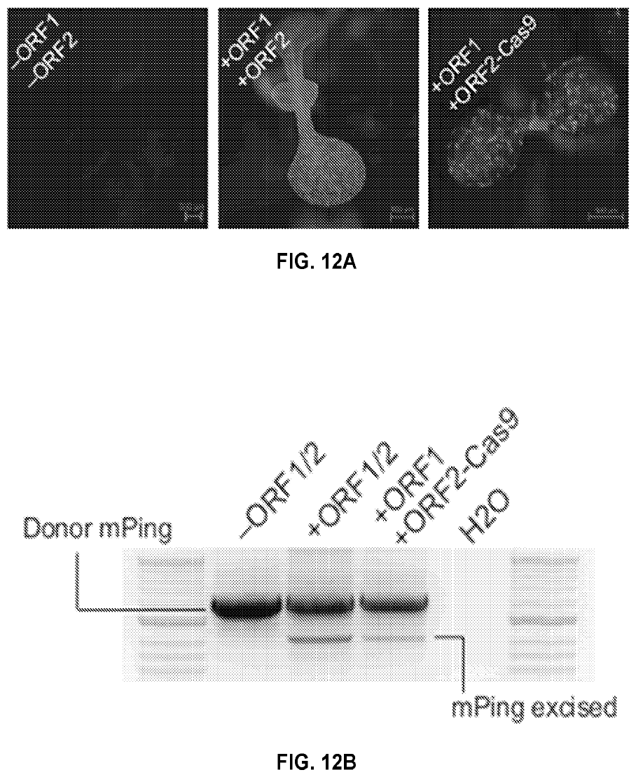

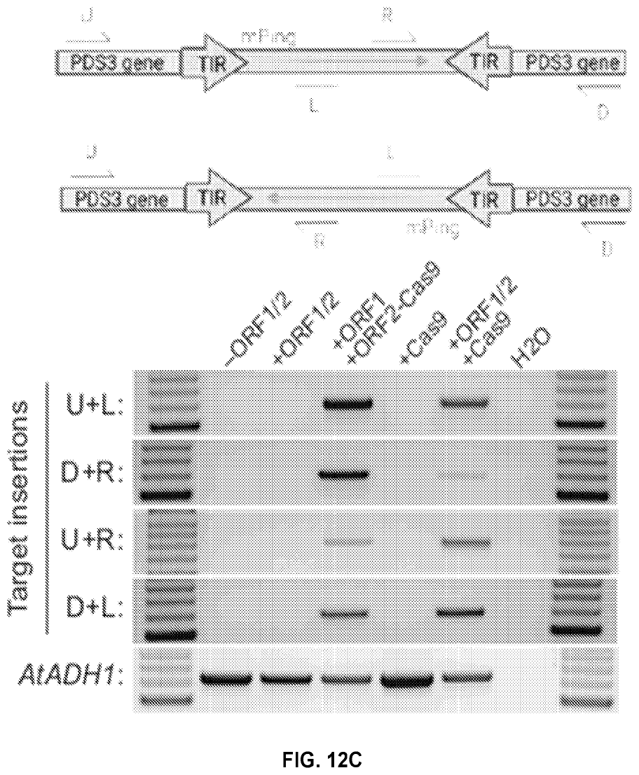

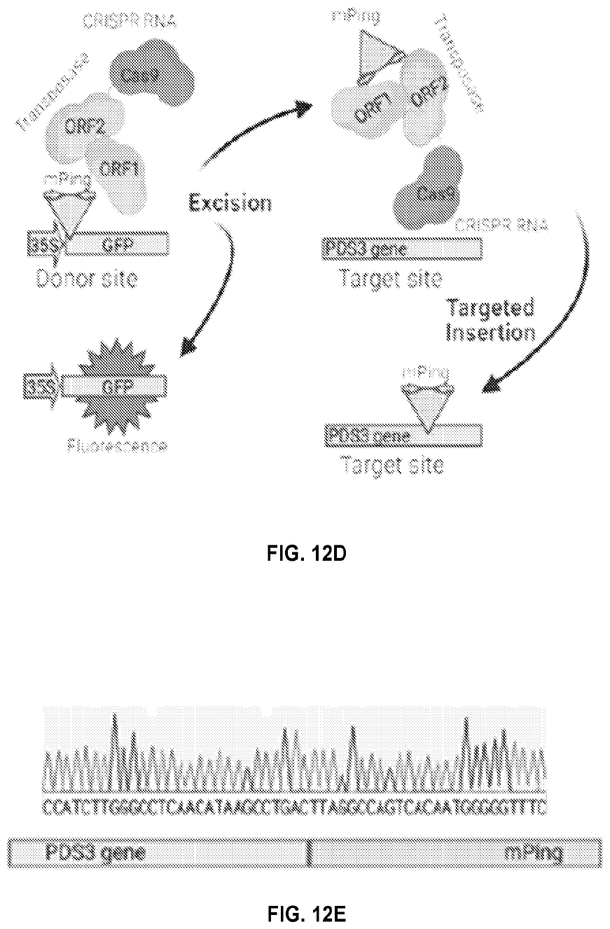

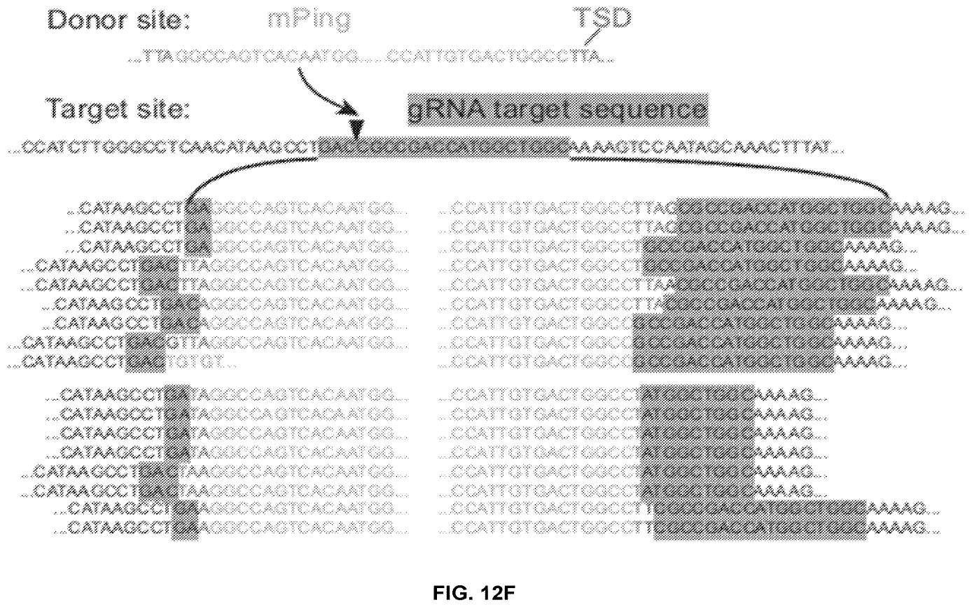

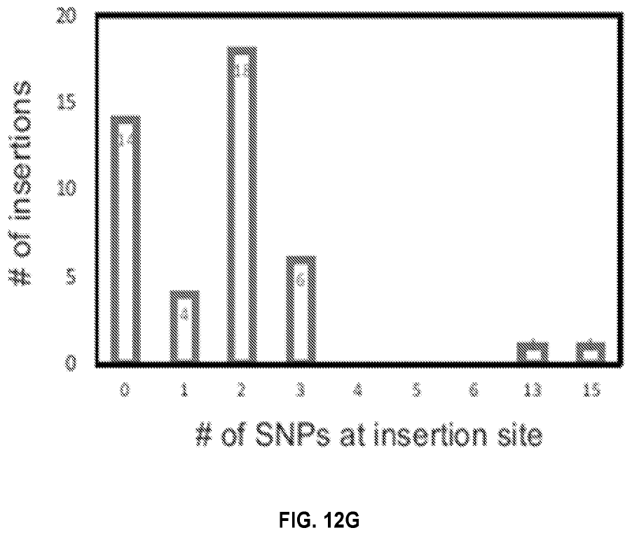

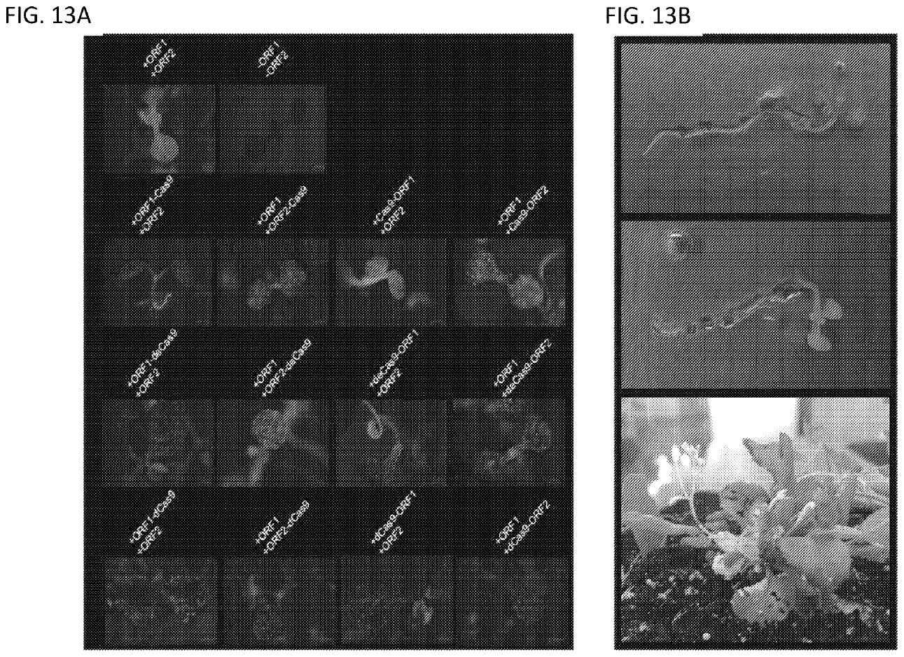

To determine if the Pong transposase was functional when linked to Cas9 derivatives, mPing excision from the donor site within GFP was assayed by visualizing the GFP fluorescence of seedlings (FIG. 12A and FIG. 13A). GFP fluorescence is a marker of mPing excision from the GFP donor site, and this fluorescence was detected for all 12 fusion proteins, but not the negative control without ORF1/ORF2 (summarized in FIG. 12A, full data in FIG. 13A), verifying that ORF1 and ORF2 are co-creating a functional transposase protein even while linked to Cas9. The function of the transposase was additionally verified using a PCR assay to detect mPing excision from the donor site. mPing excises out of its donor position when the transposase is linked to Cas9 (FIG. 12B), although the frequency may be decreased compared to transposase proteins with no fusion (FIG. 12B). A functional CRISPR/Cas9 system was verified through the observation of white seedlings and sectors in plants with the Cas9 proteins (dCas9 plants did not display white plants or sectors) (FIG. 13B). These white sectors and plants are generated by CRISPR/Cas9 targeted mutation of the PDS3 target region. Overall, these results demonstrate that fusion of the Cas9 and transposase proteins does not stop either the function of Cas9 nor the transposase.

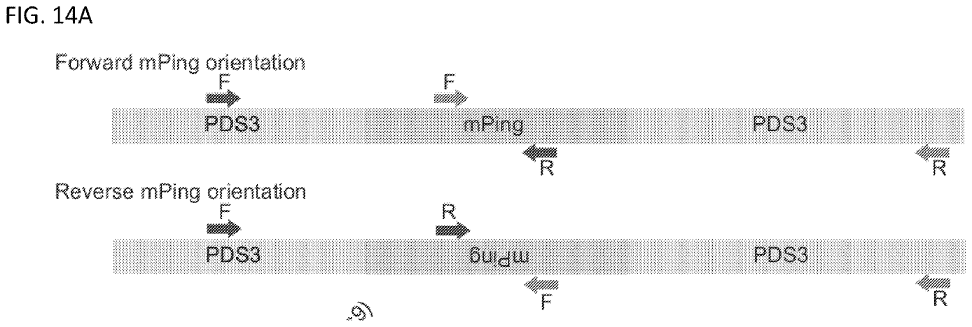

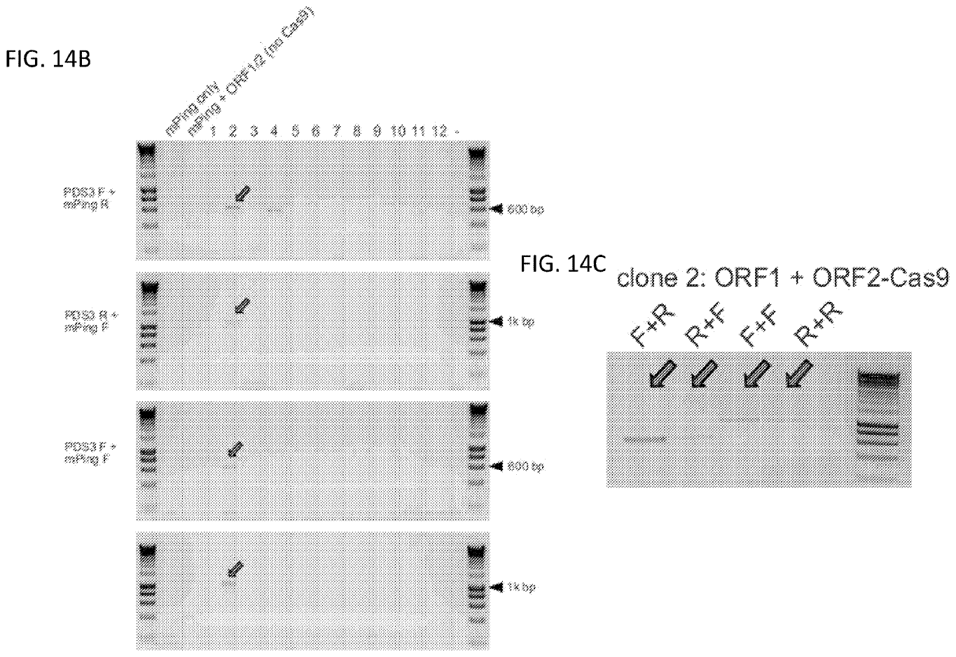

A PCR amplification strategy was employed to detect targeted mPing insertions into the Arabidopsis PDS3 gene (summarized in FIG. 12C, full data in FIGS. 14A-14B). As controls, T2 seedling pools were screened using negative control lines that either lack ORF1/ORF2, or that lack the Cas9 protein. Based on the strict expectations regarding the size of the PCR product that corresponds to the precise insertion of mPing into PDS3 (black arrowheads, FIG. 14B), it was found that clone #2 displayed the correct size PCR band in all PCR assays (FIG. 12C, FIG. 14B, FIG. 14C). This targeted insertion was only detected if both the transposase proteins (ORF1/ORF2) and Cas9 were in the same plants (FIG. 12C and FIG. 14B). The PCR can identify mPing insertions in the forward or reverse orientation (FIG. 14A), and the fact that clone #2 amplified for both suggested that there is more than one mPing insertion in this pool of plants. Clone #2 encodes for ORF1+ORF2-Cas9, where ORF2 has a C-terminal fusion to the Cas9 protein. This data demonstrated targeted insertion of mPing into the PDS3 gene (summarized in FIG. 12D), and since the catalytically-dead dCas9 version tested does not show targeted insertion, this demonstrated that the cleavage activity of Cas9 is required for targeted insertion of mPing when used with the combination of elements of the system described in this example.

### Characterization of Target Site Insertions

To characterize the sequence at the junction of the targeted insertion site, the target-site PCR assay was biologically replicated (FIG. 14C), these PCR products were cloned and sequenced using Sanger sequencing. An example of the Sanger sequencing junction of mPing and PDS3 at a targeted integration event is shown in FIG. 12E. A total of 96 clones was sequenced and found that they represented at least 44 unique targeted transposition events. Both mPing forward and reverse orientation insertions were identified, demonstrating the random directionality of the targeted insertion event (FIG. 12F). Most insertions have either intact or partial TTA/TAA sequence on one end of the insertion (FIG. 12F). This sequence came from the donor site and is part of the known target site duplication (TSD) of the Pong/mPing TE system. The presence of only one TSD, rather than one on either side of the TE insertion, as usual for a transposable element duplication event, signifies that Cas9 created a blunt cut at the insertion site, but the transposase protein made a staggered (sticky-end) cut at the donor site, before the integration event. This demonstrates that both the Cas9 and transposase proteins are functional and necessary for generating this targeted insertion: the transposase cuts mPing out from the donor site using a staggered cut with a TTA/TAA overhang on one side, and Cas9 cuts the insertion site guided by the gRNA sequence.

For each insertion, the gRNA target sequence was preserved and mPing had inserted at the expected Cas9 cleavage point between the third and fourth nucleotide (FIG. 12F). In all but one sequence read the mPing element is complete, with only small base insertions or deletions found at the target site. Of the 44 distinct insertion events, most (95%) had 0-3 nucleotide changes compared to the expected insertion junction (FIG. 12G), and 32% had perfect seamless junctions without any SNPs (FIG. 12G). The lack of deletions or other insertions at these insertion sites demonstrated the seamless or near-seamless repair of the insertion events by the transposase protein compared to typical sites of blunt-end DNA breaks.

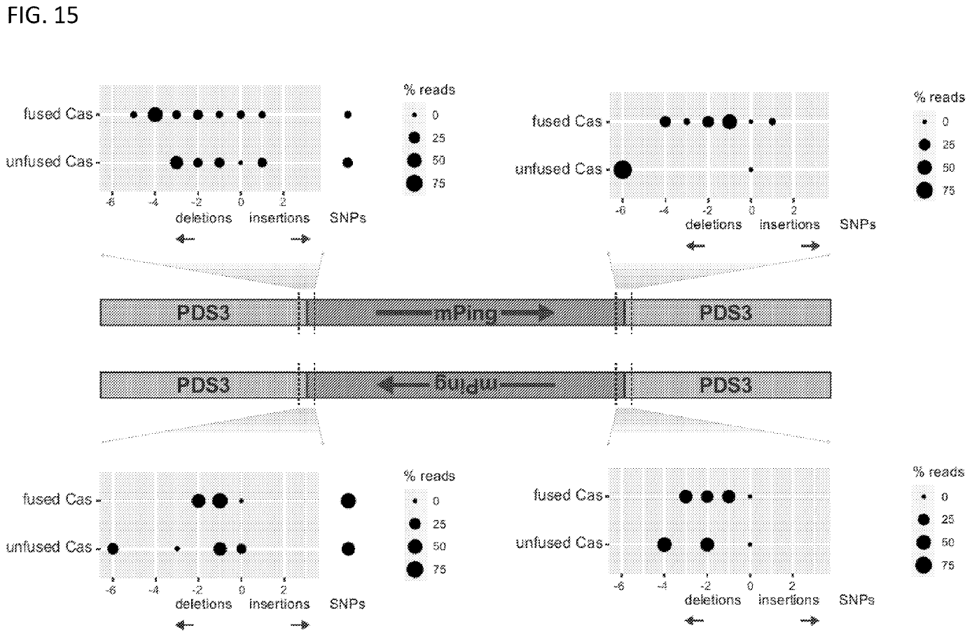

To better characterize the insertion site junctions upon targeted integration of mPing, mPing targeted integration events were deep sequenced. As shown in FIG. 15, nearly all insertions had between 0-3 nucleotide changes at the junction of mPing and the target site DNA compared to the predicted insertion configuration. The number of base deletions and insertions at the 5′ and 3′ junctions of mPing inserted into PDS3 was assayed, and since mPing can insert in either orientation, this provided four junctions for analysis (FIG. 15). When the transposase ORF2 was translationally linked to Cas9 (as in FIG. 11), it was found 0-1 base insertions, and 0-5 base deletions, however, the majority of the deletions are 0-3 bases (FIG. 15). Together, this data demonstrated that upon targeted integration of mPing, the junctions were either seamless (zero base insertions or deletions) or just a few nucleotide bases away (near-seamless). This low rate of change during targeted insertion was likely due to the transposase protein stabilizing and protecting the cleaved ends of mPing DNA and the insertion site DNA from nucleases during the integration event.

### Not Random Integration

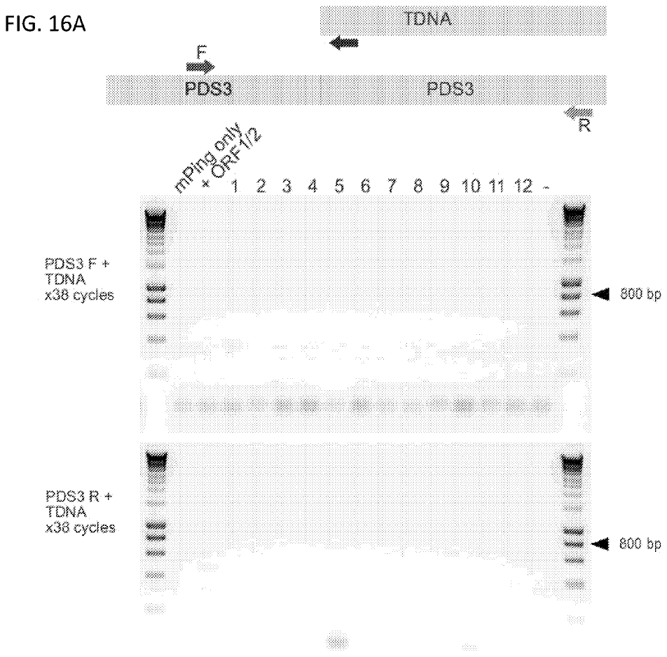

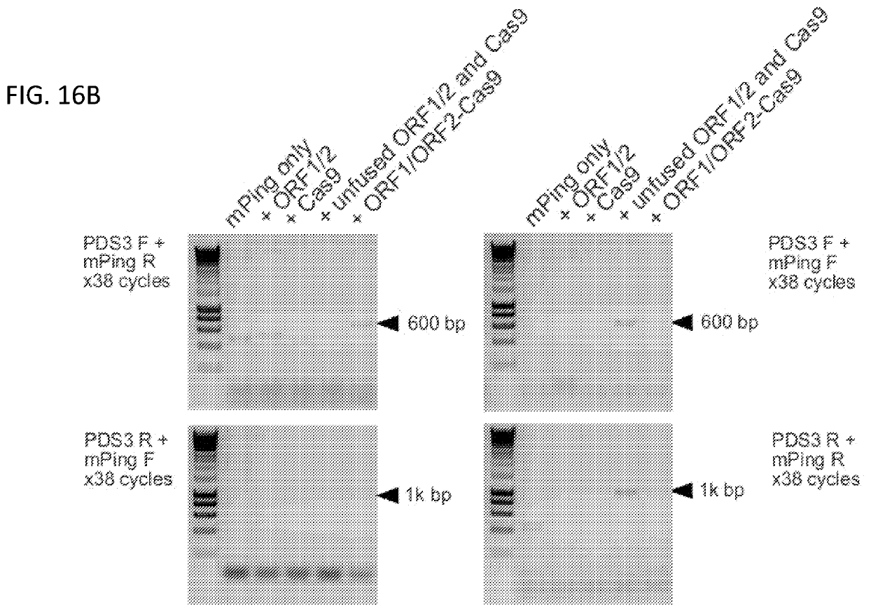

Several previous reports have demonstrated that transgenes will insert at a low frequency into any site of double-strand break. This is likely due to the transgene being extra-chromosomal DNA at the time of repair of a double-strand DNA break caused by Cas9. To determine if the mPing targeted insertion detected in FIGS. 12-14 requires the transposase protein, a PCR assay was performed for the integration of the transgene backbone encoding the ORF2-Cas9 protein into the DNA break generated at PDS3. It was reasoned that if the mPing insertion into PDS3 was a product of transgene insertion, rather than specifically transposition, it would be equally likely to detect other parts of our transgene at this insertion site location. However, the transgene sequences at PDS3 was not detected (FIG. 16A), demonstrating that mPing insertion required the transposase to excise the mPing element from the donor position to participate in targeted integration.

Next it was determined whether it was essential that the transposase protein and Cas9 were directly linked, or if both proteins unlinked in the same cell could perform targeted insertion. The findings were that in some cases the two proteins could be unlinked and targeted insertion would take place (FIG. 16B and FIG. 12C). At the same time, both transposase proteins (ORF1 and ORF2) were required and that the catalytic activity of Cas9 was necessary (FIG. 16B and FIG. 12C). Together, this data demonstrated that to obtain targeted insertion, it was essential that the transposase excise the element out of the donor position, and that Cas9 cleave the insertion site, but the two proteins do not necessarily need to be linked together. The success of the unlinked configuration of Cas9 and ORF2 suggested that any extra-chromosomal DNA can be used by the cell to repair a double-stranded break caused by Cas9, and the transposase provided this available extra-chromosomal DNA by excising mPing out of the chromosome.

The accuracy of the integration events was compared when Cas9 was linked to ORF2 compared to when the two proteins where unlinked and in the same cell (FIG. 15). In three of the four mPing junctions analyzed by deep sequencing, the unlinked ORF2/Cas9 configuration had larger 4-6 base deletions compared to the linked ORF2-Cas9 (FIG. 15). This was likely due to the more rapid binding of the transposase protein to the site that just underwent Cas9 cleavage when the two proteins are physically linked. This more rapid binding will protect free ends of DNA from degradation by nucleases. This data also suggested a key advantage of fusing Cas9 to ORF2: more accurate insertions at the single base pair resolution.

### Programmability of Target Sites

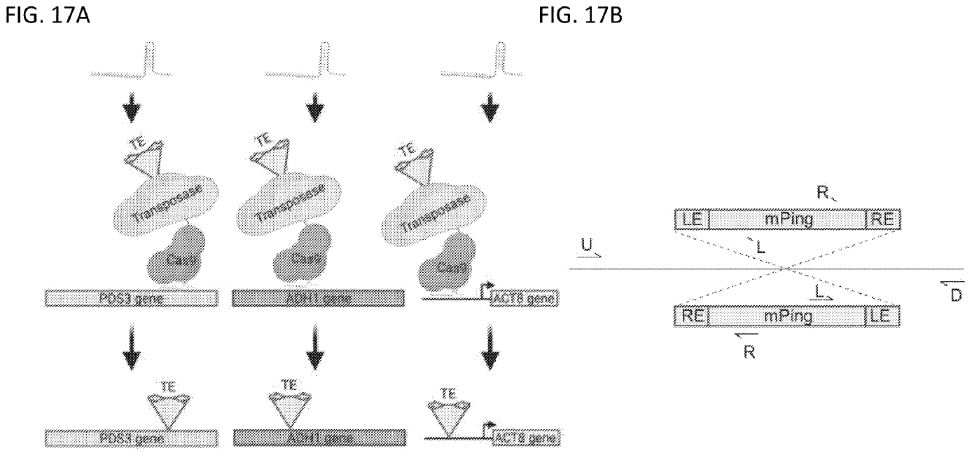

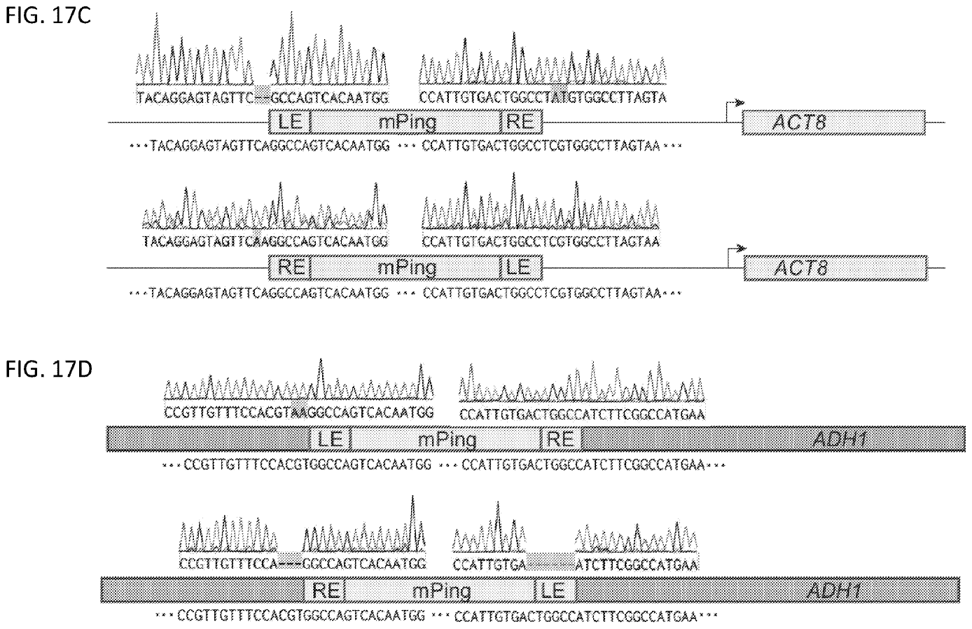

Multiple sites in the Arabidopsis genome have been successfully targeted where the inventors or others from the literature have demonstrated functional gRNAs (summarized in FIG. 17A). In addition to using gRNAs that target the gene body of PDS3 (FIGS. 12-16), the ADH1 gene and the region upstream of the ACT8 gene were successfully targeted. The PCR strategy to detect these insertions is shown in FIG. 17B. These were either within genes (PDS3 and ADH1) (ADH1 insertion shown in FIG. 17D), or in non-coding promoter regions of the ACT8 gene (shown in FIG. 17C). This data demonstrated the programmability of the targeted insertion system (summarized in FIG. 17A), as all needs to do to target a different region of the genome was to change the CRISPR gRNA sequence.

### Measurement of Frequency of Targeted Insertion

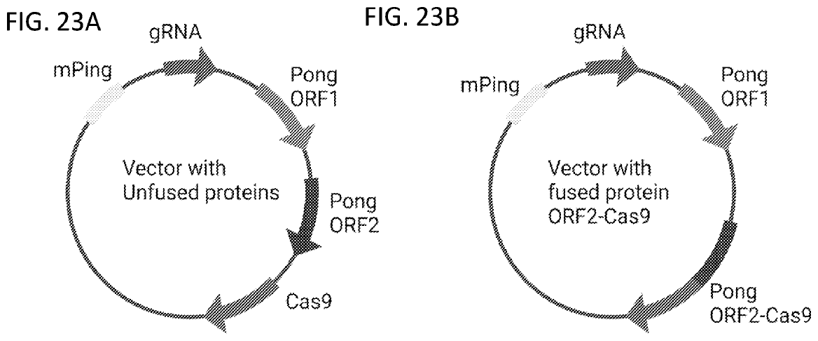

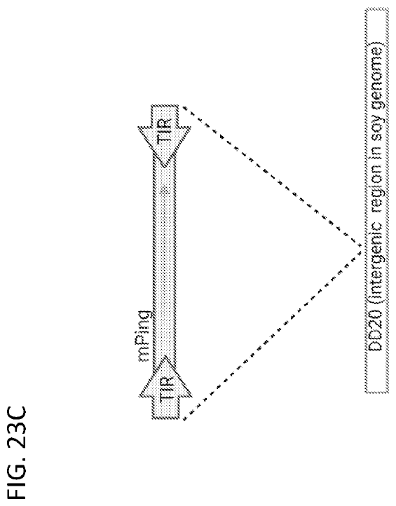

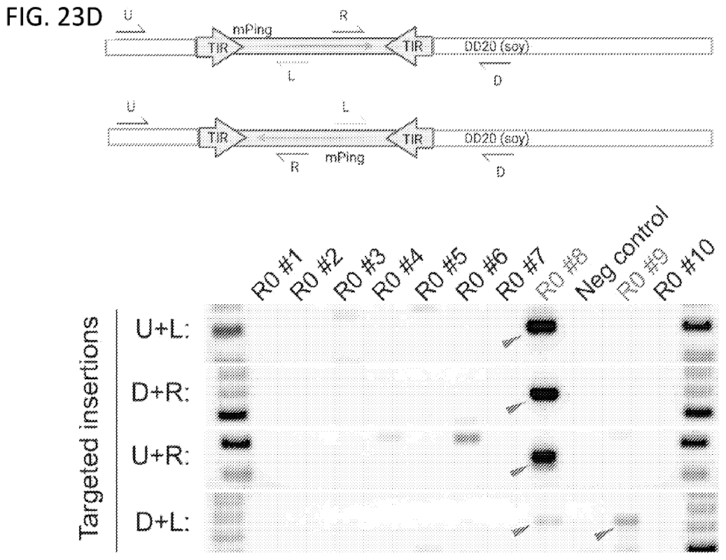

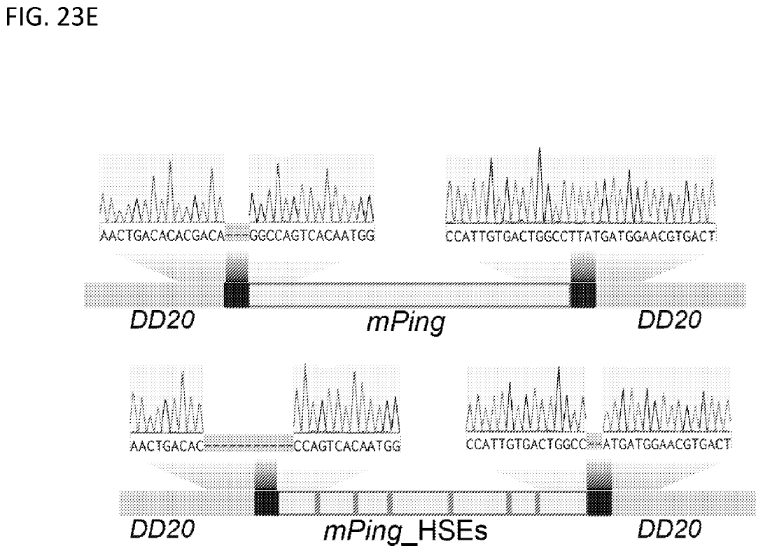

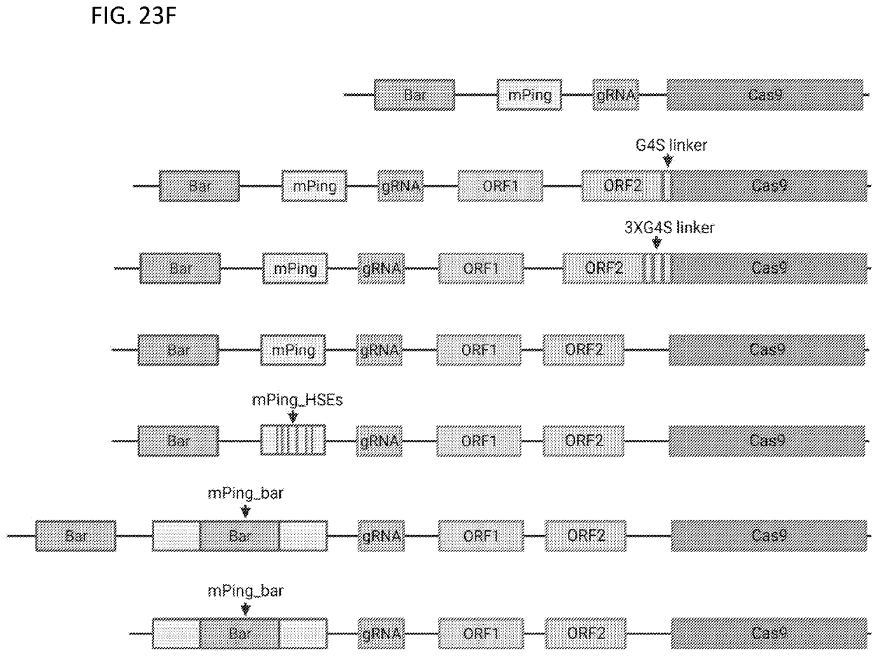

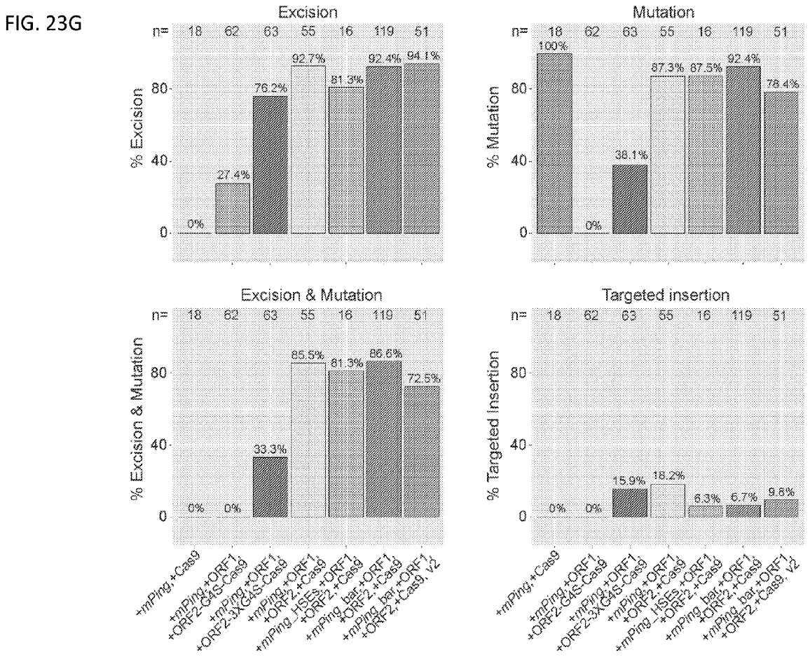

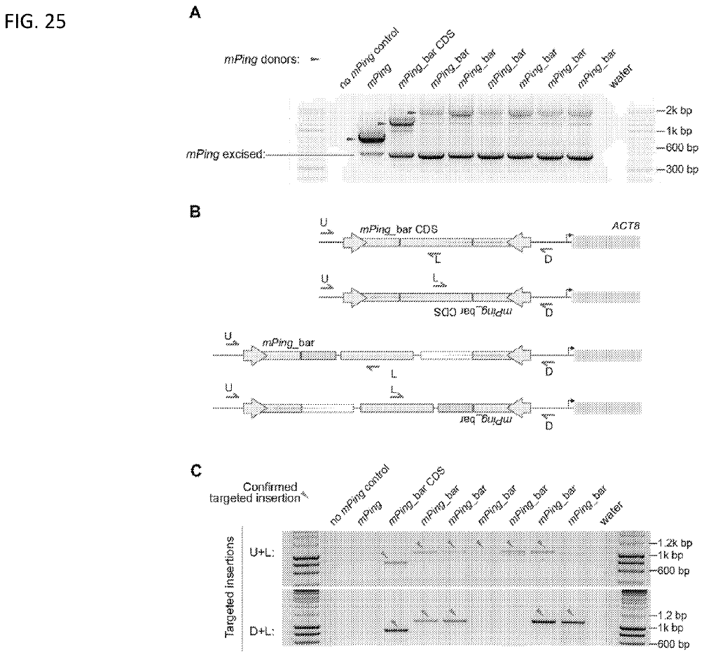

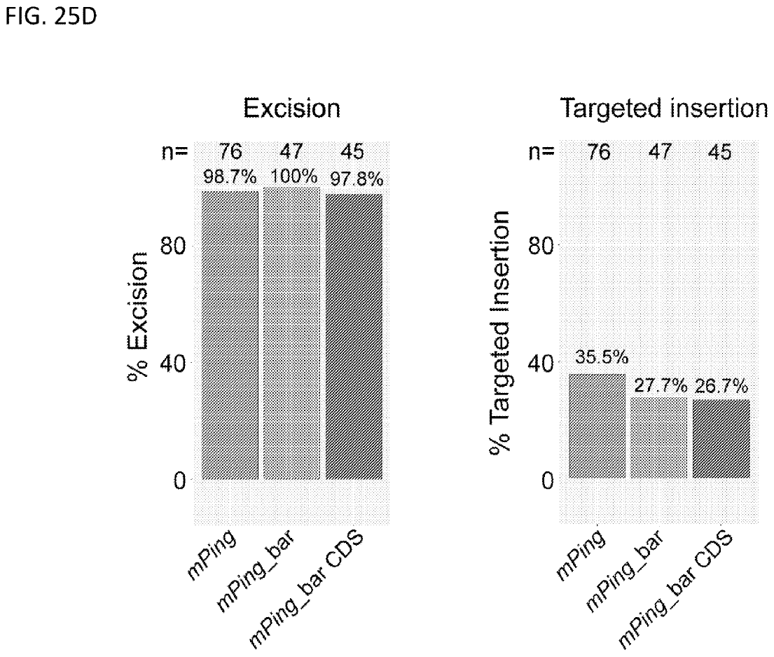

Since insertions into PDS3 generate albino plants and are lethal, insertions into the ACT8 promoter were used to measure the frequency of insertion (since the insertion will not create a gene knock-out mutation that may be selected against). Both ends of the mPing element were inserted into the ACT8 in 6.7% of T2 progeny plants (FIG. 18). This rate of more than 1 successful targeted insertion in 15 plants screened is a high rate that was easily screened for during transgenesis. The frequency of targeted insertion was later measured in Arabidopsis and found rates of 35% (FIG. 25D) and in soybean rates of 15-18% (FIG. 23G).

### Alteration of Cargo DNA

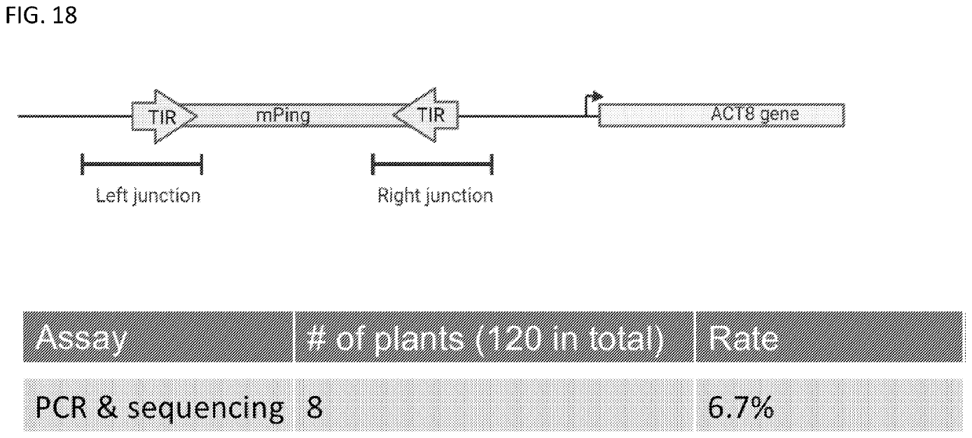

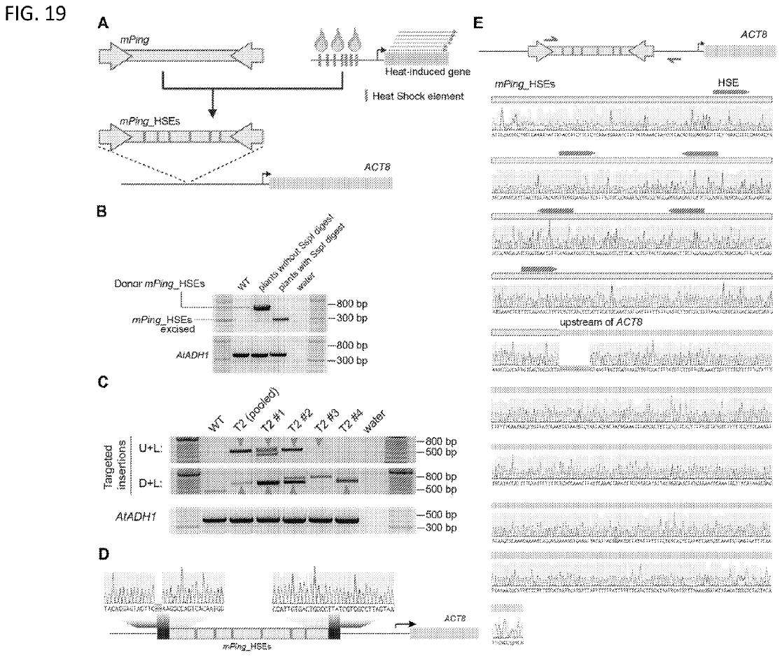

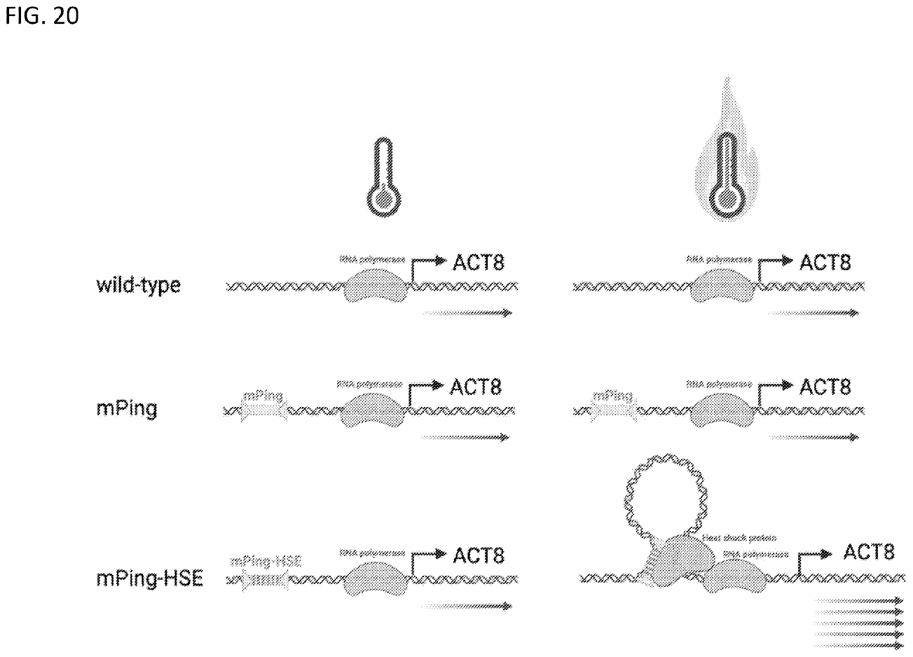

The mPing transposon is composed of terminal inverted repeats (TIRs) with DNA between them. The sequence of the TIRs is essential for transposition (as binding sites for the ORF1- and ORF2-encoded transposase proteins), but the sequence of the DNA between them (cargo) is not essential. To determine if different engineered DNA could be delivered to the target site, the cargo DNA was altered in the donor plasmid. An mPing element was engineered to carry an array of six heat-shock enhancer elements (FIG. 19A), with the goal of transposing these into a gene's promoter. A well-characterized Arabidopsis heat shock enhancer sequence was used, which is known to occur in arrays of more than one element. These enhancers were chosen because their short size and the fact that their direction upstream of a promoter did not matter, as the orientation of mPing insertion cannot be controlled. It was found that this new heat shock element-loaded mPing element (mPing-HSE) could perform the operation of a TE, as it could be excised by the transposase proteins (FIG. 19B). It was found upon transposition, mPing-HSE could successfully undergo targeted insertion similar to mPing, guided by Cas9 and the gRNA into the promoter region of the ACT8 gene (FIG. 19C), demonstrating the targeted delivery of engineered cargo DNA to a gene in its native context on the chromosome. Sanger sequencing of the junctions of mPing-HSE demonstrate a near-seamless integration with only 2 bases removed from the left junction (FIG. 19D), and in another example all six HSEs shown to be integreated into the ACT8 promoter region (FIG. 19E) demonstrating the successful delivery of these HSEs to a targeted location. In this way, this technology can alter a native gene's expression and make it heat-shock responsive (FIG. 20).

Other cargo DNA was also tested as shown in Example 13 herein below. The results show that longer DNA sequences and protein coding sequences can also be accurately and successfully delivered and inserted into genomic DNA by mPing.

### Use of Other Nucleases

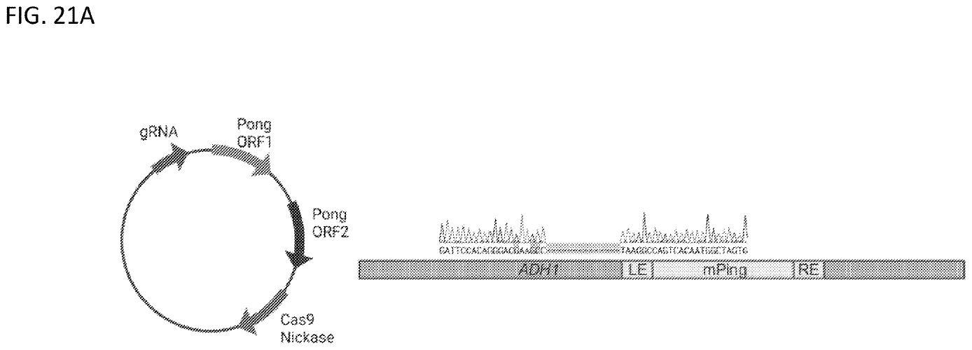

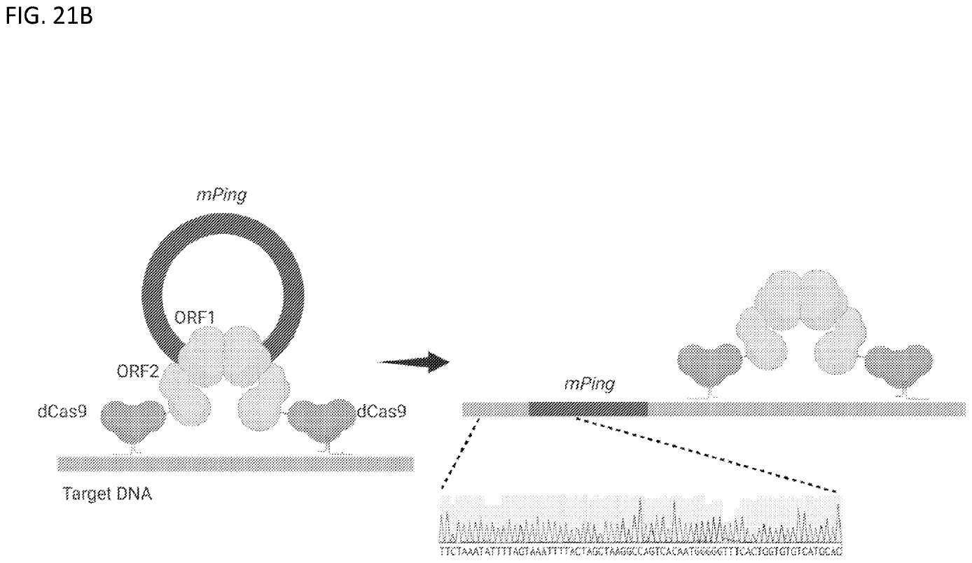

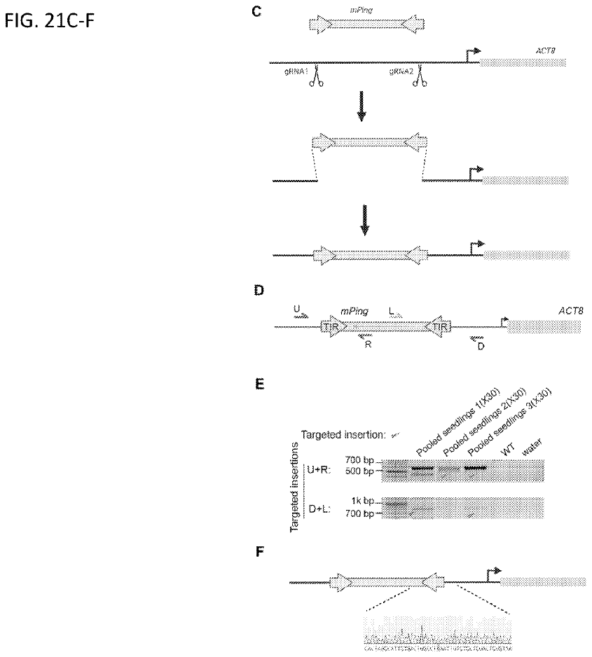

In order to determine if the system of the instant disclosure would only work with the Cas9 nuclease, or could use any sequence-specific programmable nuclease, as it was unable to detect targeted insertion with the Cas9 nickase fusion proteins created in FIG. 11. A further attempt was to detect targeted insertion with an unlinked nickase Cas9 protein in the same vector as the ORF1 and ORF2 transposase proteins (FIG. 21A). This Cas9 derivative has a mutation that results in it only cutting one strand of DNA (nicking), not both strands as the canonical Cas9. A low frequency of targeted insertion was detected using the Cas9 nickase protein. Upon Sanger sequencing this insertion displayed a 14 nucleotide deletion (FIG. 21A). This data demonstrated that other derivative versions of Cas9 can be used with transposase ORFs for targeted insertion, but since the integration site was less precise compared to Cas9, targeted insertion with the Cas9 nickase was not being pursued further.

Second, Cas9 was replaced with CFP1 nuclease, belonging to a different class of targeting nucleases, and a gRNA specific for use with CPF1 nucleases was designed. CPF1 was linked to the ORF2 transposase protein and again demonstrated successful targeted integration of mPing. This data demonstrates that the system of the instant disclosure is not specific to Cas9, and any targeted nuclease can be used. In addition, in this experiment, two gRNAs were simultaneously used in one vector and plants that had insertions in both ADH1 and the ACT8 promoter were identified. This demonstrated that two or more regions of the genome can be targeted simultaneously and efficiently. This was important for downstream multiplex engineering of more than one genome locus at a time.

Upon further experimentation, it was discovered that dCas9 could participate in targeted integration (FIG. 21B). In this case, two gRNAs were used and dCas9 linked to ORF2 to focus the transposable element to the ACT8 promoter. mPing integration at a TTA site near the sites of the gRNA targeting was observed. TTA sites are the known integration preference of mPing transposons, and this data demonstrates that dCas9 can be programmed to target a specific region of the genome for transposase-mediated integration of mPing.

Similar to the two gRNAs used in FIG. 21B, a two gRNA experiment was performed with the catatlytically active Cas9 (FIG. 21C-F). It was tested if a CRISPR-induced programmed deletion of a sequence using two gRNAs could be performed at the same time as mPing insertion, resulting in the replacement of a sequence with the targeted insertion polynucleotide (FIG. 21C). PCR was used to screen for targeted insertions (FIG. 21D-E) and Sanger sequencing confirmed the insertion (FIG. 21F). This result demonstrates that not only can this system be used for DNA addition, but also for DNA replacement and swapping of sequences in the genome.

One-Component Vs. Two-Component Systems

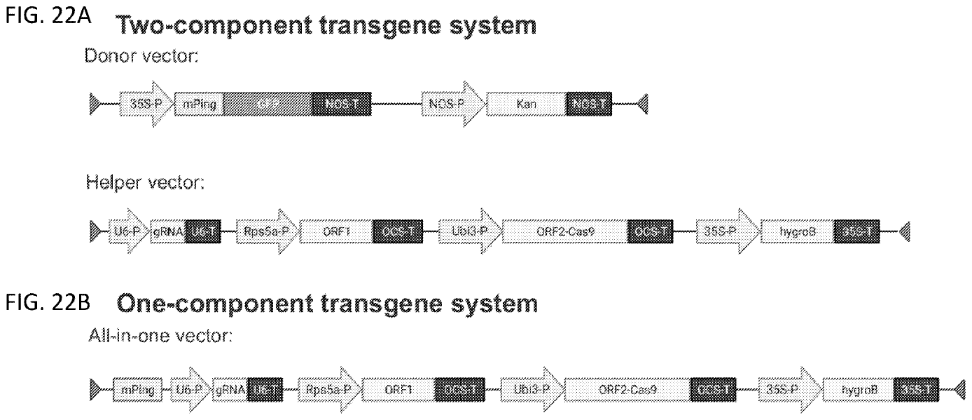

It was discovered that mPing excision and targeted insertion could take place from either the same transgene as ORF1, ORF2, Cas9 and the gRNA were encoded from (one-component system, FIG. 22B), or if the mPing donor site was already integrated into the Arabidopsis genome (two-component system) (FIG. 22A). Previous targeted insertions (FIGS. 11-16) used a 35S promoter-mPing-GFP donor site that had been previously integrated into the Arabidopsis genome (see cartoons in FIG. 10-11 and donor vector in FIG. 22A). In contrast, the mPing-HSE donor site was present on the same transgene as ORF1, ORF2, Cas9 and the gRNA are encoded from (FIG. 22B) and can still excise and undergo targeted insertion (FIG. 19A-19E). This is important because attempts to target mPing and derivative elements in other plants or with different cargo will want to use only the one-component transgene and the one cycle of transgenesis to accomplish targeted insertion. Of note, the one-component mPing donor site was not in the 35S-GFP sequence, but rather in different sequence that was used to cut down on the size of the transgene and does not provide the excision reporter of GFP fluorescence (FIGS. 22A and 22B). Instead, when using the one-component system, excision is monitored by PCR only (FIG. 19B), and this demonstrated that the surrounding DNA sequence around mPing at the donor site was not important in this system.

### Example 8: Measuring Specificity/Off-Target Integration Rate

The rate of off-target mPing insertion into the genome is tested. This is important because it is reasoned that the direct fusion between Cas9 and ORF2 has fewer off-targets compared to having the two proteins present but unlinked. Therefore, fusing the two proteins can be important to limit the activity of the transposase protein so it does not integrate mPing all over the genome.

Approaches to detect mPing insertion sites include Southern blot, PCR ‘transposable-element display’ and long-read sequencing to sequence the full genome and detect other full or partial integration events of mPing.

To improve propagation of the insertion events into the next generation and limit the off-target effect, the promoter of the Cas9-transposase fusion protein is altered to only expressed in the egg cell. Accordingly, all cells of the plant will have the same insertion that occurred in the egg cell, while the insertions will not continue to accumulate during plant development.

### Example 9: Testing Other Uses of Targeted Insertion

Repeated delivery of different transgene cargos to the same permissive location in the genome is tested. The results demonstrate the reduced variability and improved experimental/product reproducibility when transgenes are targeted to the same region of the genome using systems of the instant disclosure.

Targeted delivery of a protein tag to a coding region using systems of the instant disclosure is also tested. The protein tag can be used to epitope tag a protein at its native location and within its native regulatory context.

Targeted addition of a strong promoter to drive constitutive expression of a gene at its native position for either over-expression of the sense mRNA or antisense expression for gene silencing is also tested.

### Example 10: Rewiring Gene Regulation Based on Targeted Insertion

The mPing-HSE element was previously generated, in which the cargo DNA has an array of six heat-shock cis-regulatory enhancer elements (FIG. 19A). During the heat shock response, these enhancer elements are bound by a heat shock protein and enhance the transcription of a nearby gene. The one-component transgene system (FIG. 22B) is used to target the distal promoter region of the ACT8 gene (FIG. 19C-19E). The ACT8 gene is chosen because it is not regulated by heat and is often used as a control gene because of its steady transcription into mRNA even during heat stress (FIG. 20). The goal is to demonstrate the utility of the targeted insertion technology by rewiring the ACT8 gene in its native chromosomal context, providing this gene the new programmed ability to increase expression as a response to heat stress. Lines with the original mPing (no heat-shock elements) inserted at the same location are used as controls (insertion in FIG. 19, experimental design in FIG. 20). An additional control is wild-type plants without any insertion upstream of ACT8. Both of these controls do not to provide ACT8 with higher expression during heat shock (FIG. 20).

### Example 12: Targeted Insertion in a Crop

A variation of the systems of the instant disclosure was transformed into soybean plants (Glycine max). Soybean is annually one of the top three crops grown in the United States, and the #1 oil crop. Transformation was performed by the Danforth Center's Plant Transformation Facility (PTF). Soybean explants were transformed using Agrobacterium, cultured, and selected for the integration of the transgene. Next, roots and shoots were regenerated and the plants transplanted to soil and sampled.

To transfer the system to soybeans, a binary vector that is proven to function in soybean transformation was used. The transgenes all have the same mPing and ORF1 sequences, and a different gRNA that has been previously demonstrated to function in the soybean genome, which targets an intergenic region called “DD20” (PMID 26294043). Two configurations of the transgene system were used in soybean: 1) ORF2 unlinked to Cas9 (FIG. 23A), and 2) ORF2 linked to Cas9 (FIG. 23B).

R0 plants that have been regenerated from the transformation process were screened and confirmed via PCR to have the entire transgene integrated into the genome. Plants were assayed for mPing excision which demonstrates the successful transposition of the donor polynucleotide, Cas9 cleavage and mutation of the target locus (demonstrates that the CRISPR/Cas parts of the system are working), and for targeted insertion of mPing (see below). Screening for targeted insertion was performed using four PCR reactions that target each end of the mPing insertion, in either direction of potential insertion (FIG. 23C-23D).

Of the 10 transgenic R0 plants produced from the unlinked transgene configuration in FIG. 23A, two amplified in our assays for targeted insertion of mPing (Plant #8 and #9, FIG. 23D). These PCR products were sequenced and confirmed to be targeted integrations of mPing at the DD20 intergenic target locus (top of FIG. 23E). This rate of 20% of R0 plants is very high compared to other methods of crop genome targeted integration or HDR. Of note, since plant #8 amplifies in all four PCR reactions (FIG. 23D), it represents more than one insertion event.

The identified targeted insertion event of mPing is a near-seamless insertion on the 3′ side, and has a 10 base pair deletion on the 5′ end. This deletion is all of soybean DD20 DNA, while the mPing insertion is identical to mPing at the donor site. This again demonstrates that the mutations, if they do occur, are in the target site DNA, and not in the newly transposed element.

Additional constructs for transformation and testing in soybean were generated (FIG. 23F). A total of 62 R0 plants were investigated with the ORF2-Cas9 linked protein in FIG. 23G. Even with considerable effort, a targeted insertion in these plants was not identified. It was found that ~27% of these plants have mPing excision, demonstrating that the transposase aspect of our system is working, but none of these plants showed mutation accumulation at the target site, which demonstrates that Cas9 was not functional when linked to ORF2 in soybean plants. The linkage that was used to fuse ORF2 to Cas9 was a single copy of the G4S flexible linker (SEQ ID NO: 64). This was the same linker that was functional to fuse ORF2 to Cas9 in Arabidopsis. We then tested if a longer flexible linker of 3× copies of the G4S linker could functionally fuse ORF2 to Cas9 in soybean (constructs on FIG. 23F). The 3×G4S construct has similar mPing excision as the unlinked Cas9+ORF2 configuration, and targeted insertions for the 3×G4S linker construct was a high 15.9% (FIG. 23G), which again is an improvement over other methods of targeted insertion in the soybean genome.

### Example 13: Targeted Insertion of an Expression Construct for Expressing a Protein

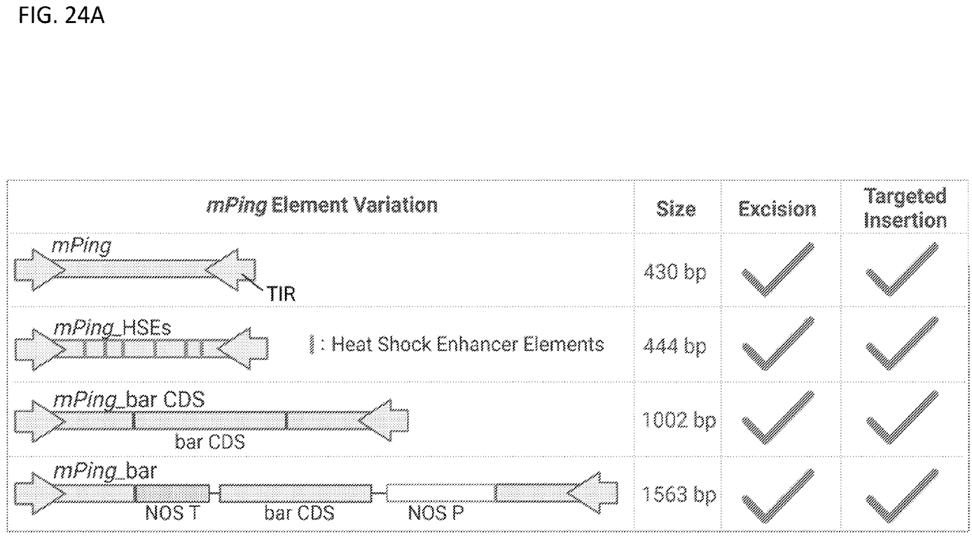

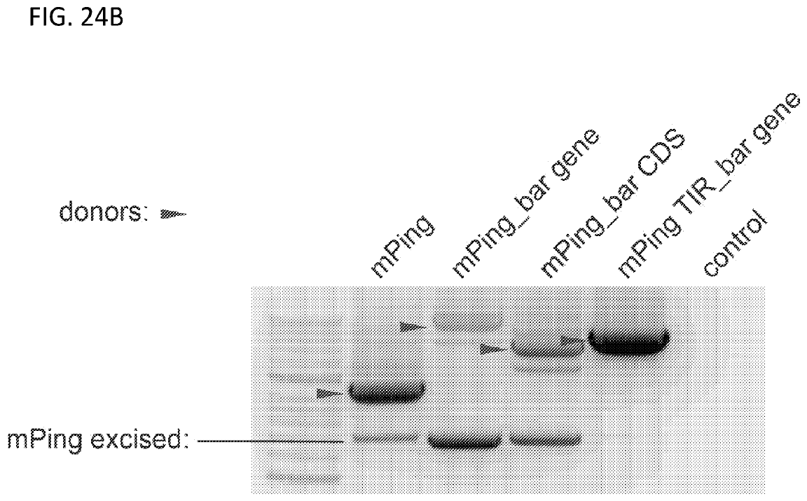

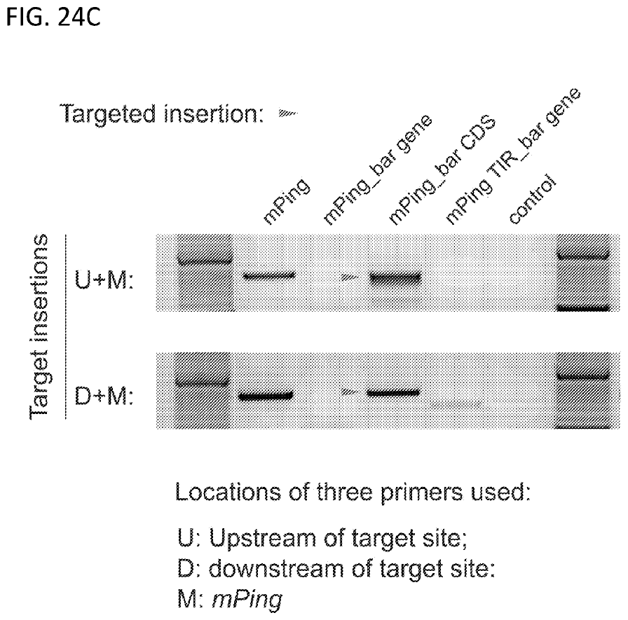

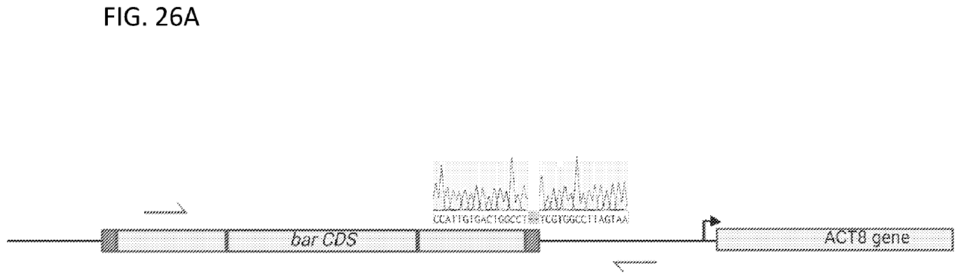

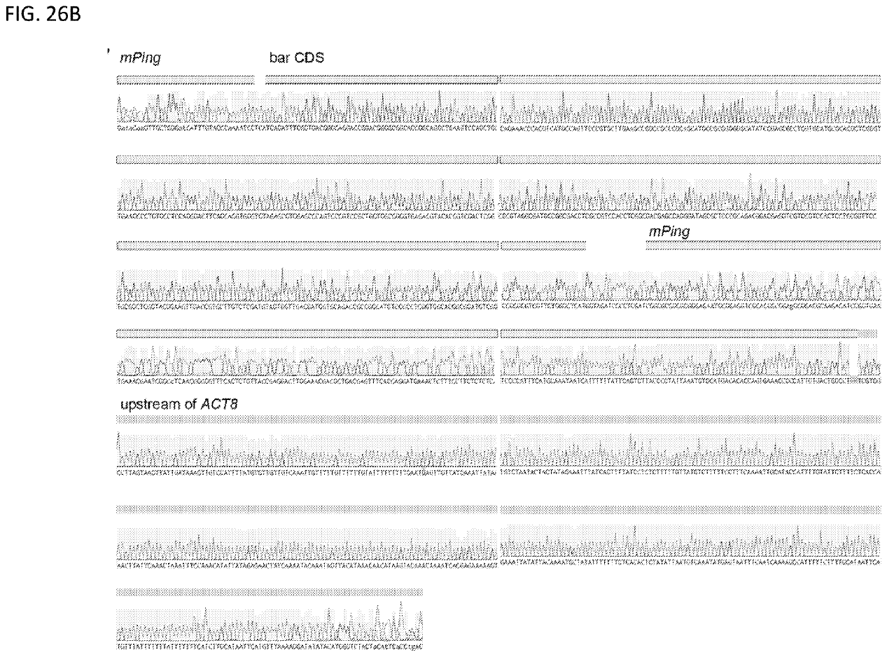

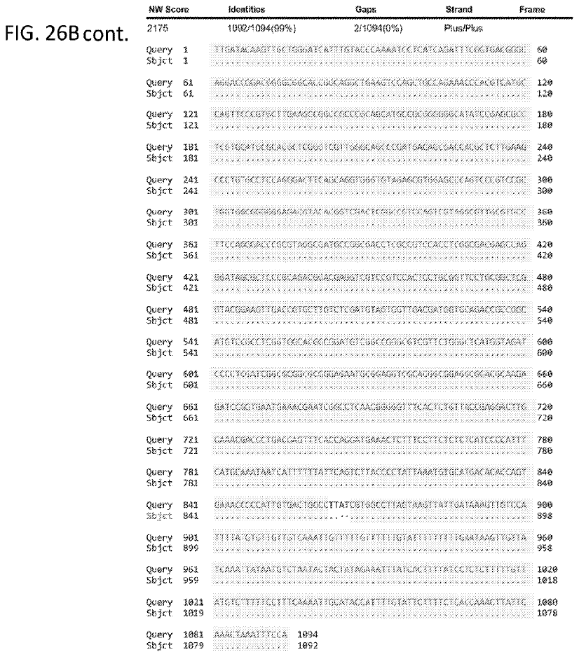

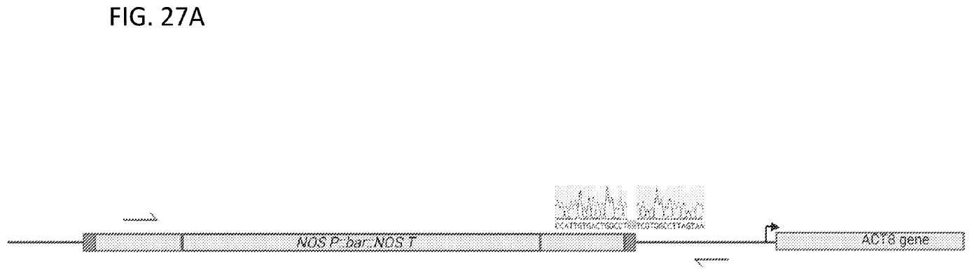

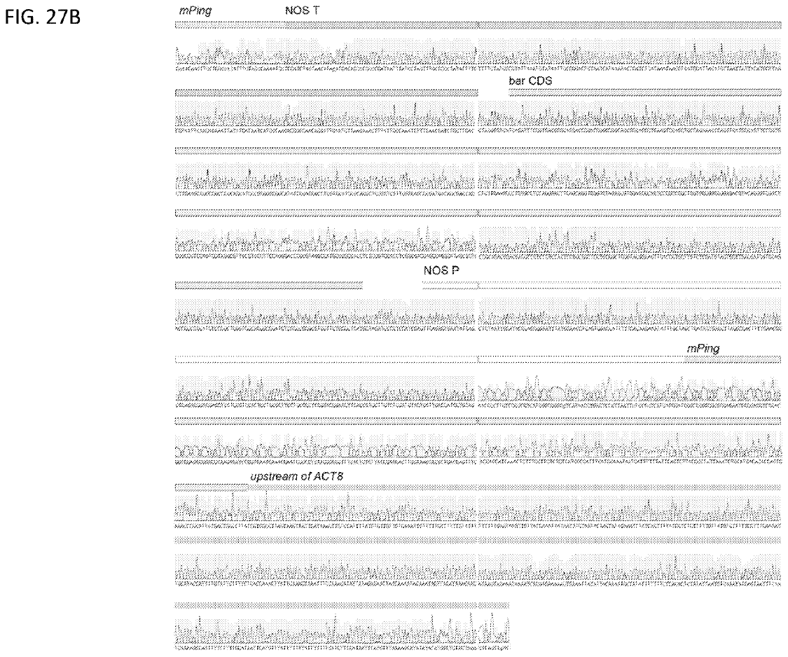

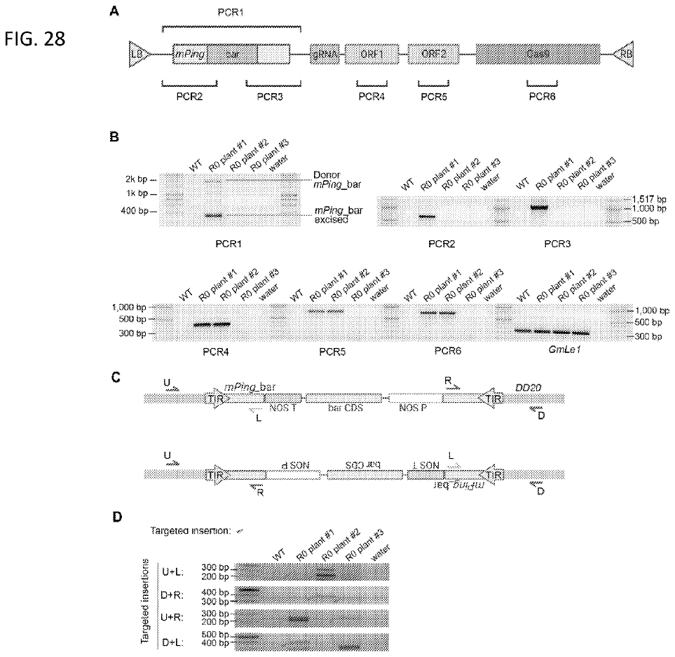

This experiment tested different cargo nucleic acid constructs to be delivered via transposase-mediated target site integration in soybean (FIG. 23F-G) and Arabidopsis thaliana (FIG. 24A). To test the cargo capacity that can be delivered by mPing, the rice 430 bp mPing element (FIG. 24A first construct; SEQ ID NO: 96) was used as a control. This control 430 bp mPing control is capable of excision and targeted insertion into the region upstream of the Arabidopsis ACT8 gene and to the DD20 site in Soybean. Second, larger cargos were tested by cloning the protein-coding region of the herbicide bialaphos resistance gene (bar) into mPing, creating a 1 kb synthetic element (FIG. 24A third construct; SEQ ID NO: 98). Third, the bar gene (PMID: 16453790) was cloned into mPing, including the bar promoter and terminator elements (FIG. 24A second construct; SEQ ID NO: 97), generating a 1.5 kb element. Both of these elements were capable of excision (FIG. 24B and FIG. 25A), and successfully targeted insertion of both elements into the non-coding region upstream of the Arabidopsis ACT8 gene was confirmed (FIGS. 24C, 25B, 26A, 26B, 27A and 27B). Sequencing confirmed that the entire bar gene cassette was delivered intact and mutation-free to the targeted insertion site (FIGS. 26B and 27B). In Arabidopsis the frequency of targeted insertion of mPing-bar and mPing-bar CDS was compared to mPing, and there is a small reduction in frequency (FIG. 25D), but the rate of targeted insertion is overall higher than 25% for all of these mPing variations. In soybean, only the 1.5 kb mPing-bar construct was tested (not the ORF-only construct) (FIG. 23F 6th construct; SEQ ID NO: 97), generating a 1.5 kb element, and it was able to both excise and be integrated to the targeted location (FIG. 23G). To test if this expression cassette driving the bar resistance gene was functional in soybean, a transgene was constructed where the only herbicide resistance gene in the vector was present within mPing (bottom construct in FIG. 23F). In soybean plants, this mPing-bar element confers herbicide resistance, as plants could be recovered after transformation and grown on media with herbicide added. mPing-bar undergoes excision and targeted insertion (FIG. 23G). Some of the resulting regenerated soybean plants have mPing-bar at the DD20 targeted insertion site, but lack the bar gene at the transgene (genotyped in FIG. 28A-28B). Some plants have mPing-bar at the targeted insertion location and a partial transgene integration (plant #2 in FIG. 28B-28D), while others have only the targeted insertion and no transgene (plant #3 in FIG. 28B-28D). These plants are herbicide resistant, and therefore the herbicide resistance of these plants must be driven off the only copy of the bar gene, which is located in mPing at the DD20 targeted insertion site.

### Example 14: TIRs of mPing are not Sufficient for Efficient Transposition

The above bar gene insertions was within an otherwise complete mPing element (SEQ ID NO: 96). To test if the TIRs of mPing are sufficient for transposition, or if other sequences within the mPing element are also necessary, the bar gene was surrounded with 33 bp mPing TIRs (generating ‘mPing TIR_bar’; SEQ ID NO: 99; FIG. 24A, fourth construct) and found that this generated an extremely low excision efficiency compared with the original mPing (FIG. 24B). No targeted insertion was detected for mPing TIR_bar (FIG. 24C). This result demonstrates that the mPing TIRs are not sufficient for efficient transposition and suggests that other sequences within mPing enhance transposition.

### Example 15: Cas9 Integrated in Plant Genome

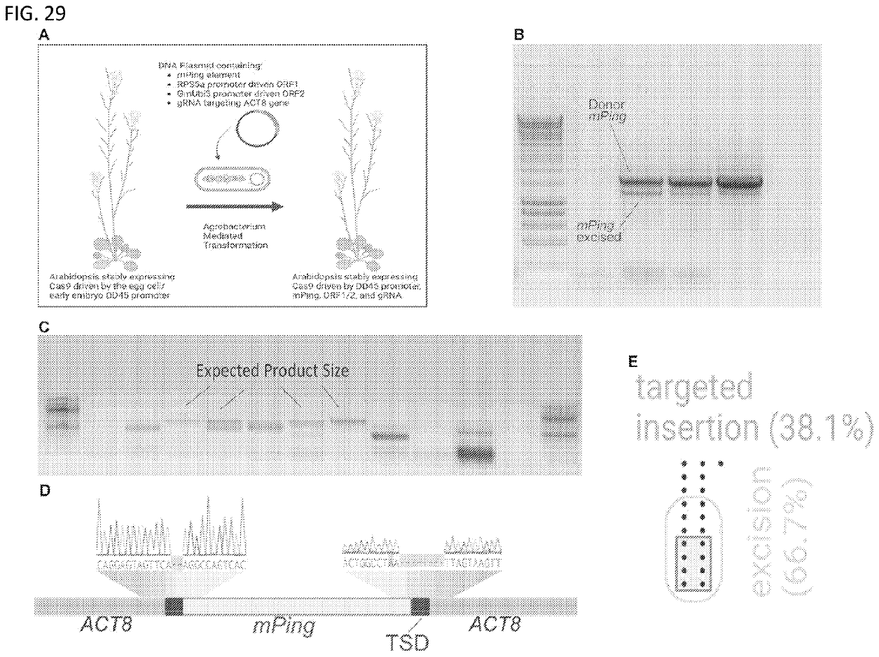

A variation of the systems of the instant disclosure wherein the targeting nuclease was a Cas9 protein expressed from an expression construct stably integrated into the genome of Arabidopsis was also successfully generated (FIG. 29A). The expression construct expresses Cas9 under the control of the DD45 embryo promoter. The Arabidopsis plants were transformed with a construct comprising an mPing cargo element, an expression construct for expressing a gRNA targeting the mPing cargo to the ACT8 gene, and expression constructs expressing Pong ORF1+ORF2 to achieve targeted insertion. FIG. 29B shows that the system was capable of excision of the mPing cargo, and FIG. 29C shows that the system was capable of targeted integration of of the mPing cargo into the target nucleic acid locus in the ACT8 gene. Sanger sequencing show that mPing was successfully inserted in ACT8 (FIG. 29D). The rate of excision was 66.7% and the rate of integration was 38.1% (FIG. 29E). This result demonstrates that the engineered system can be expressed at different cell types and different times in development.

### Sequences

| SEQ. ID | | |
|---|---|---|
| NO. | Sequence | Name |
| 1 | MDPSPAVDPSPAVDPSPAAETRRRATGKGGKQRGGKQLGLKRPPPI | Pong ORF1 |
| | SVPATPPPAATSSSPAAPTAIPPRPPQSSPIFVPDSPNPSPAAPTS | protein |
| | SLASGTSTARPPQPQGGGWGPTSTISPNFASFFGNQQDPNSCLVRG | |
| | YPPGGFVNFIQQNCPPQPQQQGENFHFVGHNMGENPISPQPPSAYG | |
| | TPTPQATNQGTSTNIMIDEEDNNDDSRAAKKRWTHEEEERLASAWL | |
| | NASKDSIHGNDKKGDTFWKEVTDEFNKKGNGKRRREINQLKVHWSR | |
| | LKSAISEFNDYWSTVTQMHTSGYSDDMLEKEAQRLYANREGKPFAL | |
| | VHWWKILKREPKWCAQFEKRKRKSEMDAVPEQQKRPIGREAAKSER | |
| | KRKRKKENVMEGIVLLGDNVQKIIKVTQDRKLEREKVTEAQIHISN | |
| | VNLKAAEQQKEAKMFEVYNSLLTQDTSNMSEEQKARRDKALQKLEE | |
| | KLFAD* | |
| 2 | atggatccgtcgccggccgtggatccgtcgccggccgtggatccgt | DNA |
| | cgccggctgctgaaacccggcggcgtgcaaccgggaaaggaggcaa | sequence |
| | acagcgcgggggcaagcaactaggattgaagaggccgccgccgatt | encoding |
| | tctgtcccggccaccccgcctcctgctgcgacgtcttcatcccctg | Pong ORF1 |
| | ctgcgccgacggccatcccaccacgaccaccgcaatcttcgccgat | protein |
| | tttcgtccccgattcgccgaatccgtcaccggctgcgccgacctcc | |
| | tctcttgcttcggggacatcgacggcaaggccaccgcaaccacaag | |
| | gaggaggatggggaccaacatcgaccatttccccaaactttgcatc | |
| | tttctttggaaaccaacaagacccaaattcatgtttggtcaggggt | |
| | tatcctccaggagggtttgtcaattttattcaacaaaattgtccgc | |
| | cgcagccacaacagcaaggtgaaaattttcatttcgttggtcacaa | |
| | tatggggttcaacccaatatctccacagccaccaagtgcctacgga | |
| | acaccaacaccccaagctacgaaccaaggcacttcaacaaacatta | |
| | tgattgatgaagaggacaacaatgatgacagtagggcagcaaagaa | |
| | aagatggactcatgaagaggaagagagactggccagtgcttggttg | |
| | aatgcttctaaagactcaattcatgggaatgataagaaaggtgata | |
| | cattttggaaggaagtcactgatgaatttaacaagaaagggaatgg | |
| | aaaacgtaggagggaaattaaccaactgaaggttcactggtcaagg | |
| | ttgaagtcagcgatctctgagttcaatgactattggagtacggtta | |
| | ctcaaatgcatacaagcggatactcagacgacatgcttgagaaaga | |
| | ggcacagaggctgtatgcaaacaggtttggaaaaccttttgcgttg | |
| | gtccattggtggaagatactcaaaagagagcccaaatggtgtgctc | |
| | agtttgaaaagaggaaaaggaagagcgaaatggatgctgttccaga | |
| | acagcagaaacgtcctattggtagagaagcagcaaagtctgagcgc | |
| | aaaagaaagcgcaagaaagaaaatgttatggaaggcattgtcctcc | |
| | taggggacaatgtccagaaaattatcaaagtgacgcaagatcggaa | |
| | gctggagcgtgagaaggtcactgaagcacagattcacatttcaaac | |
| | gtaaatttgaaggcagcagaacagcaaaaagaagcaaagatgtttg | |
| | aggtatacaattccctgctcactcaagatacaagtaacatgtctga | |
| | agaacagaaggctcgccgagacaaggcattacaaaagctggaggaa | |
| | aagttatttgctgactag | |
| 3 | MQSLAISLLLSETHSLESHTKTSSLLSLLFLSSSKMSEQNTDGSQV | Pong ORF2 |
| | PVNLLDEFLAEDEIIDDLLTEATVVVQSTIEGLQNEASDHRHHPRK | protein |
| | HIKRPREEAHQQLVNDYFSENPLYPSKIFRRRERMSRPLFLRIVEA | |
| | LGQWSVYFTQRVDAVNRKGLSPLQKCTAAIRQLATGSGADELDEYL | |
| | KIGETTAMEAMKNFVKGLQDVFGERYLRRPTMEDTERLLQLGEKRG | |
| | FPGMFGSIDCMHWHWERCPVAWKGQFTRGDQKVPTLILEAVASHDL | |
| | WIWHAFFGAAGSNNDINVLNQSTVFIKELKGQAPRVQYMVNGNQYN | |
| | TGYFLADGIYPEWAVFVKSIRLPNTEKEKLYADMQEGARKDIERAF | |
| | GVLQRRFCILKRPARLYDRGVLRDVVLACIILHNMIVEDEKETRII | |
| | EEDADANVPPSSSTVQEPEFSPEQNTPEDRVLEKDISIRDRAAHNR | |
| | LKKDLVEHIWNKFGGAAHRTGN | |
| 4 | atgcagagtttagccatctctctactcctctcagaaactcattccc | DNA |
| | tcttttctcatacgaagacctcctcccttttatctttactgtttct | sequence |
| | ctcttcttcaaagatgtctgagcaaaatactgatggaagtcaagtt | encoding |
| | ccagtgaacttgttggatgagttcctggctgaggatgagatcatag | Pong ORF2 |
| | atgatcttctcactgaagccacggtggtagtacagtccactataga | protein |
| | aggtcttcaaaacgaggcttctgaccatcgacatcatccgaggaag | |
| | cacatcaagaggccacgagaggaagcacatcagcaactGgtgaatg | |
| | attacttttcagaaaatcctctttacccttccaaaatttttcgtcg | |
| | aagatttcgtatgtctaggccactttttcttcgcatcgttgaggca | |
| | ttaggccagtggtcagtgtatttcacacaaagggtggatgctgtta | |
| | atcggaaaggactcagtccactgcaaaagtgtactgcagctattcg | |
| | ccagttggctactggtagtggcgcagatgaactagatgaatatctg | |
| | aagataggagagactacagcaatggaggcaatgaagaattttgtca | |
| | aaggtcttcaagatgtgtttggtgagaggtatcttaggcgccccac | |
| | tatggaagataccgaacggcttctccaacttggtgagaaacgtggt | |
| | tttcctggaatgttcggcagcattgactgcatgcactggcattggg | |
| | aaagatgcccagtagcatggaagggtcagttcactcgtggagatca | |
| | gaaagtgccaaccctgattcttgaggctgtggcatcgcatgatctt | |
| | tggatttggcatgcattttttggagcagcgggttccaacaatgata | |
| | tcaatgtattgaaccaatctactgtatttatcaaggagctcaaagg | |
| | acaagctcctagagtccagtacatggtaaatgggaatcaatacaat | |
| | actgggtattttcttgctgatggaatctaccctgaatgggcagtgt | |
| | ttgttaagtcaatacgactcccaaacactgaaaaggagaaattgta | |
| | tgcagatatgcaagaaggggcaagaaaagatatcgagagagccttt | |
| | ggtgtattgcagcgaagattttgcatcttaaaacgaccagctcgtc | |
| | tatatgatcgaggtgtactgcgagatgttgttctagcttgcatcat | |
| | acttcacaatatgatagttgaagatgagaaggaaaccagaattatt | |
| | gaagaagatgcagatgcaaatgtgcctcctagttcatcaaccgttc | |
| | aggaacctgagttctctcctgaacagaacacaccatttgatagagt | |
| | tttagaaaaagatatttctatccgagatcgagcggctcataaccga | |
| | cttaagaaagatttggtggaacacatttggaataagtttggtggtg | |
| | ctgcacatagaactggaaat | |
| 5 | APKKKRKVGIHGVPAADKKYSIGLDIGTNSVGWAVITDEYKVPSKK | Cas 9 |
| | FKVLGNTDRHSIKKNLIGALLFDSGETAEATRLKRTARRRYTRRKN | protein |
| | RICYLQEIFSNEMAKVDDSFFHRLEESELVEEDKKHERHPIFGNIV | |
| | DEVAYHEKYPTIYHLRKKLVDSTDKADLRLIYLALAHMIKERGHEL | |
| | IEGDLNPDNSDVDKLFIQLVQTYNQLFEENPINASGVDAKAILSAR | |
| | LSKSRRLENLIAQLPGEKKNGLFGNLIALSLGLTPNFKSNFDLAED | |
| | AKLQLSKDTYDDDLDNLLAQIGDQYADLFLAAKNLSDAILLSDILR | |
| | VNTEITKAPLSASMIKRYDEHHQDLTLLKALVRQQLPEKYKEIFFD | |
| | QSKNGYAGYIDGGASQEEFYKFIKPILEKMDGTEELLVKLNREDLL | |
| | RKQRTFDNGSIPHQIHLGELHAILRRQEDFYPELKDNREKIEKILT | |
| | FRIPYYVGPLARGNSRFAWMTRKSEETITPWNFEEVVDKGASAQSF | |
| | IERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGV | |
| | EDRFNASLGTYHDLLKIIKDKDELDNEENEDILEDIVLTLTLFEDR | |
| | EMIEERLKTYAHLFDDKVMKQLKRRRYTGWGRLSRKLINGIRDKQS | |
| | GKTILDFLKSDGFANRNFMQLIHDDSLTFKEDIQKAQVSGQGDSLH | |
| | EHIANLAGSPAIKKGILQTVKVVDELVKVMGRHKPENIVIEMAREN | |
| | QTTQKGQKNSRERMKRIEEGIKELGSQILKEHPVENTQLQNEKLYL | |
| | YYLQNGRDMYVDQELDINRLSDYDVDHIVPQSFLKDDSIDNKVLTR | |
| | SDKNRGKSDNVPSEEVVKKMKNYWRQLLNAKLITQRKEDNLTKAER | |
| | GGLSELDKAGFIKRQLVETRQITKHVAQILDSRMNTKYDENDKLIR | |
| | EVKVITLKSKLVSDERKDFQFYKVREINNYHHAHDAYLNAVVGTAL | |
| | IKKYPKLESEFVYGDYKVYDVRKMIAKSEQEIGKATAKYFFYSNIM | |
| | NFFKTEITLANGEIRKRPLIETNGETGEIVWDKGRDFATVRKVLSM | |
| | PQVNIVKKTEVQTGGFSKESILPKRNSDKLIARKKDWDPKKYGGFD | |
| | SPTVAYSVLVVAKVEKGKSKKLKSVKELLGITIMERSSFEKNPIDE | |
| | LEAKGYKEVKKDLIIKLPKYSLFELENGRKRMLASAGELQKGNELA | |
| | LPSKYVNFLYLASHYEKLKGSPEDNEQKQLFVEQHKHYLDEIIEQI | |
| | SEFSKRVILADANLDKVLSAYNKHRDKPIREQAENIIHLFTLTNLG | |
| | APAAFKYFDTTIDRKRYTSTKEVLDATLIHQSITGLYETRIDLSQL | |
| | GGDKRPAATKKAGQAKKKK* | |
| 6 | gctccgaagaagaagaggaaggttggcatccacggggtgccagctg | Cas 9 DNA |
| | ctgacaagaagtactcgatcggcctcgatattgggactaactctgt | |
| | tggctgggccgtgatcaccgacgagtacaaggtgccctcaaagaag | |
| | ttcaaggtcctgggcaacaccgatcggcattccatcaagaagaatc | |
| | tcattggcgctctcctgttcgacagcggcgagacggctgaggctac | |
| | gcggctcaagcgcaccgcccgcaggcggtacacgcgcaggaagaat | |
| | cgcatctgctacctgcaggagattttctccaacgagatggcgaagg | |
| | ttgacgattctttcttccacaggctggaggagtcattcctcgtgga | |
| | ggaggataagaagcacgagcggcatccaatcttcggcaacattgtc | |
| | gacgaggttgcctaccacgagaagtaccctacgatctaccatctgc | |
| | ggaagaagctcgtggactccacagataaggcggacctccgcctgat | |
| | ctacctcgctctggcccacatgattaagttcaggggccatttcctg | |
| | atcgagggggatctcaacccggacaatagcgatgttgacaagctgt | |
| | tcatccagctcgtgcagacgtacaaccagctcttcgaggagaaccc | |
| | cattaatgcgtcaggcgtcgacgcgaaggctatcctgtccgctagg | |
| | ctctcgaagtctcggcgcctcgagaacctgatcgcccagctgccgg | |
| | gcgagaagaagaacggcctgttcgggaatctcattgcgctcagcct | |
| | ggggctcacgcccaacttcaagtcgaatttcgatctcgctgaggac | |
| | gccaagctgcagctctccaaggacacatacgacgatgacctggata | |
| | acctcctggcccagatcggcgatcagtacgcggacctgttcctcgc | |
| | tgccaagaatctgtcggacgccatcctcctgtctgatattctcagg | |
| | gtgaacaccgagattacgaaggctccgctctcagcctccatgatca | |
| | agcgctacgacgagcaccatcaggatctgaccctcctgaaggcgct | |
| | ggtcaggcagcagctccccgagaagtacaaggagatcttcttcgat | |
| | cagtcgaagaacggctacgctgggtacattgacggcggggcctctc | |
| | aggaggagttctacaagttcatcaagccgattctggagaagatgga | |
| | cggcacggaggagctgctggtgaagctcaatcgcgaggacctcctg | |
| | aggaagcagcggacattcgataacggcagcatcccacaccagattc | |
| | atctcggggagctgcacgctatcctgaggaggcaggaggacttcta | |
| | ccctttcctcaaggataaccgcgagaagatcgagaagattctgact | |
| | ttcaggatcccgtactacgtcggcccactcgctaggggcaactccc | |
| | gcttcgcttggatgacccgcaagtcagaggagacgatcacgccgtg | |
| | gaacttcgaggaggtggtcgacaagggcgctagcgctcagtcgttc | |
| | atcgagaggatgacgaatttcgacaagaacctgccaaatgagaagg | |
| | tgctccctaagcactcgctcctgtacgagtacttcacagtctacaa | |
| | cgagctgactaaggtgaagtatgtgaccgagggcatgaggaagccg | |
| | gctttcctgtctggggagcagaagaaggccatcgtggacctcctgt | |
| | tcaagaccaaccggaaggtcacggttaagcagctcaaggaggacta | |
| | cttcaagaagattgagtgcttcgattcggtcgagatctctggcgtt | |
| | gaggaccgcttcaacgcctccctggggacctaccacgatctcctga | |
| | agatcattaaggataaggacttcctggacaacgaggagaatgagga | |
| | tatcctcgaggacattgtgctgacactcactctgttcgaggaccgg | |
| | gagatgatcgaggagcgcctgaagacttacgcccatctcttcgatg | |
| | acaaggtcatgaagcagctcaagaggaggaggtacaccggctgggg | |
| | gaggctgagcaggaagctcatcaacggcattcgggacaagcagtcc | |
| | gggaagacgatcctcgacttcctgaagagcgatggcttcgcgaacc | |
| | gcaatttcatgcagctgattcacgatgacagcctcacattcaagga | |
| | ggatatccagaaggctcaggtgagcggccagggggactcgctgcac | |
| | gagcatatcgcgaacctcgctggctcgccagctatcaagaagggga | |
| | ttctgcagaccgtgaaggttgtggacgagctggtgaaggtcatggg | |
| | caggcacaagcctgagaacatcgtcattgagatggcccgggagaat | |
| | cagaccacgcagaagggccagaagaactcacgcgagaggatgaaga | |
| | ggatcgaggagggcattaaggagctggggtcccagatcctcaagga | |
| | gcacccggtggagaacacgcagctgcagaatgagaagctctacctg | |
| | tactacctccagaatggccgcgatatgtatgtggaccaggagctgg | |
| | atattaacaggctcagcgattacgacgtcgatcatatcgttccaca | |
| | gtcattcctgaaggatgactccattgacaacaaggtcctcaccagg | |
| | tcggacaagaaccggggcaagtctgataatgttccttcagaggagg | |
| | tcgttaagaagatgaagaactactggcgccagctcctgaatgccaa | |
| | gctgatcacgcagcggaagttcgataacctcacaaaggctgagagg | |
| | ggcgggctctctgagctggacaaggcgggcttcatcaagaggcagc | |
| | tggtcgagacacggcagatcactaagcacgttgcgcagattctcga | |
| | ctcacggatgaacactaagtacgatgagaatgacaagctgatccgc | |
| | gaggtgaaggtcatcaccctgaagtcaaagctcgtctccgacttca | |
| | ggaaggatttccagttctacaaggttcgggagatcaacaattacca | |
| | ccatgcccatgacgcgtacctgaacgcggtggtcggcacagctctg | |
| | atcaagaagtacccaaagctcgagagcgagttcgtgtacggggact | |
| | acaaggtttacgatgtgaggaagatgatcgccaagtcggagcagga | |
| | gattggcaaggctaccgccaagtacttcttctactctaacattatg | |
| | aatttcttcaagacagagatcactctggccaatggcgagatccgga | |
| | agcgccccctcatcgagacgaacggcgagacgggggagatcgtgtg | |
| | ggacaagggcagggatttcgcgaccgtcaggaaggttctctccatg | |
| | ccacaagtgaatatcgtcaagaagacagaggtccagactggcgggt | |
| | tctctaaggagtcaattctgcctaagcggaacagcgacaagctcat | |
| | cgcccgcaagaaggactgggatccgaagaagtacggcgggttcgac | |
| | agccccactgtggcctactcggtcctggttgtggcgaaggttgaga | |
| | agggcaagtccaagaagctcaagagcgtgaaggagctgctggggat | |
| | cacgattatggagcgctccagcttcgagaagaacccgatcgatttc | |
| | ctggaggcgaagggctacaaggaggtgaagaaggacctgatcatta | |
| | agctccccaagtactcactcttcgagctggagaacggcaggaagcg | |
| | gatgctggcttccgctggcgagctgcagaaggggaacgagctggct | |
| | ctgccgtccaagtatgtgaacttcctctacctggcctcccactacg | |
| | agaagctcaagggcagccccgaggacaacgagcagaagcagctgtt | |
| | cgtcgagcagcacaagcattacctcgacgagatcattgagcagatt | |
| | tccgagttctccaagcgcgtgatcctggccgacgcgaatctggata | |
| | aggtcctctccgcgtacaacaagcaccgcgacaagccaatcaggga | |
| | gcaggctgagaatatcattcatctcttcaccctgacgaacctcggc | |
| | gcccctgctgctttcaagtacttcgacacaactatcgatcgcaaga | |
| | ggtacacaagcactaaggaggtcctggacgcgaccctcatccacca | |
| | gtcgattaccggcctctacgagacgcgcatcgacctgtctcagctc | |
| | gggggcgacaagcggccagcggcgacgaagaaggcggggcaggcga | |
| | agaagaagaagtga | |
| 7 | GGCCAGTCACAA | mPing |
| | | terminal |
| | | inverted |
| | | repeat 1 |
| 8 | TTGTGACTGGCC | mPing |
| | | terminal |
| | | inverted |
| | | repeat 2 |
| 9 | TTAGGCCAGTCACAA | Sequence |
| | | at |
| | | insertion |
| | | site |
| 10 | TTGTGACTGGCCTTA | Sequence |
| | | at |
| | | insertion |
| | | site |
| 11 | CCATCTTGGGCCTCAACATAAGCCTGACCGCCGACCATGGCTGGCA | gRNA |
| | AAAGTCCAATAGCAAACTTTAT | targeting |
| | | site in |
| | | PDS 3 and |
| | | surrounding |
| | | sequence. |
| 12 | CATAAGCCTGAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 13 | TTGTGACTGGCCTTAGCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 14 | CATAAGCCTGAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 15 | TTGTGACTGGCCTTAGCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 16 | CATAAGCCTGAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 17 | TTGTGACTGGCCTGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 18 | CATAAGCCTGACTTAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 19 | TTGTGACTGGCCTGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 20 | CATAAGCCTGACTTAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 21 | TTGTGACTGGCCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 22 | CATAAGCCTGACAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 23 | TTGTGACTGGCCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 24 | CATAAGCCTGACAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 25 | TTGTGACTGGCCTTAACCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 26 | CATAAGCCTGACGTTAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 27 | TTGTGACTGGCCTTACGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 28 | CATAAGCCTGACTGTGT | Nucleic |
| | | acid |
| | | sequence |
| | | at |
| | | insertion |
| | | sites of |
| | | a unique |
| | | transposition |
| | | event of |
| | | FIG. 5 |
| 29 | TTGTGACTGGCCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 30 | CATAAGCCTGATAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 31 | TTGTGACTGGCCTATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 32 | CATAAGCCTGATAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 33 | TTGTGACTGGCCTATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 34 | CATAAGCCTGATAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 35 | TTGTGACTGGCCTATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 36 | CATAAGCCTGATAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 37 | TTGTGACTGGCCTATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 38 | CATAAGCCTGACTAAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 39 | TTGTGACTGGCCTATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 40 | CATAAGCCTGACTAAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 41 | TTGTGACTGGCCTTCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 42 | CATAAGCCTGAAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 43 | TTGTGACTGGCCTTCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 44 | CATAAGCCTGAAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 45 | TTGTGACTGGCCTTCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 46 | CATAAGCCTGACTTAAGGCCAGTCACAA | Nucleic |
| | | acid |
| | | sequence |
| | | at |
| | | insertion |
| | | sites of |
| | | a unique |
| | | transposition |
| | | event of |
| | | FIG. 5 |
| 47 | TTGTGACTGGCCTTCGCCGACCATGGCTGGCAAAAG | Nucleic |
| | | acid |
| | | sequence |
| | | at |
| | | insertion |
| | | sites of |
| | | a unique |
| | | transposition |
| | | event of |
| | | FIG. 5 |
| 48 | CAACATAAGCCTGACAGGCCAGTCACAATGG | Nucleic |
| | | acid |
| | | sequence |
| | | at |
| | | insertion |
| | | sites of |
| | | a unique |
| | | transposition |
| | | event of |
| | | FIG. 7B |
| 49 | CCATTGTGACTGGCC | Nucleic |
| | | acid |
| | | sequence |
| | | at |
| | | insertion |
| | | sites of |
| | | a unique |
| | | transposition |
| | | event of |
| | | FIG. 7B |
| 50 | GCCGACCATGGCTG | Nucleic |
| | | acid |
| | | sequence |
| | | at |
| | | insertion |
| | | sites of |
| | | a unique |
| | | transposition |
| | | event of |
| | | FIG. 7B |
| 51 | CAACATAAGCCTGAC | Nucleic |
| | | acid |
| | | sequence |
| | | at |
| | | insertion |
| | | sites of |
| | | a unique |
| | | transposition |
| | | event of |
| | | FIG. 7B |
| 52 | GGCCAGTCACAATGG | Nucleic |
| | | acid |
| | | sequence |
| | | at |
| | | insertion |
| | | sites of |
| | | a unique |
| | | transposition |
| | | event of |
| | | FIG. 7B |
| 53 | CCATTGTGACTGGCCCGCCGACCATGGCTG | Nucleic |
| | | acid |
| | | sequence |
| | | at |
| | | insertion |
| | | sites of |
| | | a unique |
| | | transposition |
| | | event of |
| | | FIG. 7B |
| 54 | CCGTTGTTTCCACGTAAGGCCAGTCACAATGG | Nucleic |
| | | acid |
| | | sequence |
| | | at |
| | | insertion |
| | | sites of |
| | | a unique |
| | | transposition |
| | | event of |
| | | FIG. 7B |
| 55 | CCATTGTGACTGGCCATCTTCGGCCATGAA | Nucleic |
| | | acid |
| | | sequence |
| | | at |
| | | insertion |
| | | sites of |
| | | a unique |
| | | transposition |
| | | event of |
| | | FIG. 7B |
| 56 | CCGTTGTTTCCACGT | Nucleic |
| | | acid |
| | | sequence |
| | | at |
| | | insertion |
| | | sites of |
| | | a unique |
| | | transposition |
| | | event of |
| | | FIG. 7B |
| 57 | GGCCAGTCACAATGG | Nucleic |
| | | acid |
| | | sequence |
| | | at |
| | | insertion |
| | | sites of |
| | | a unique |
| | | transposition |
| | | event of |
| | | FIG. 7B |
| 58 | CCATGTGACTGGCCATCTTCGGCCATGAA | Nucleic |
| | | acid |
| | | sequence |
| | | at |
| | | insertion |
| | | sites of |
| | | a unique |
| | | transposition |
| | | event of |
| | | FIG. 7B |
| 59 | TACAGGAGTAGTTC | Nucleic |
| | | acid |
| | | sequence |
| | | at |
| | | insertion |
| | | sites of |
| | | a unique |
| | | transposition |
| | | event of |
| | | FIG. 7B |
| 60 | GCCAGTCACAATGG | Nucleic |
| | | acid |
| | | sequence |
| | | at |
| | | insertion |
| | | sites of |
| | | a unique |
| | | transposition |
| | | event of |
| | | FIG. 7B |
| 61 | CCATTGTGACTGGCCTCGTGGCCTTAGTAA | Nucleic |
| | | acid |
| | | sequence |
| | | at |
| | | insertion |
| | | sites of |
| | | a unique |
| | | transposition |
| | | event of |
| | | FIG. 7B |
| 62 | TACAGGAGTAGTTCAGGCCAGTCACAATGG | Nucleic |
| | | acid |
| | | sequence |
| | | at |
| | | insertion |
| | | sites of |
| | | a unique |
| | | transposition |
| | | event of |
| | | FIG. 7B |
| 63 | CCATTGTGACTGGCCTCGTGGCCTTAGTAA | Nucleic |
| | | acid |
| | | sequence |
| | | at |
| | | insertion |
| | | sites of |
| | | a unique |
| | | transposition |
| | | event of |
| | | FIG. 7B |
| 64 | GSSSS | G4S |
| | | flexible |
| | | protein |
| | | linker |
| 65 | GCCAGCCATGGTCGGCGGTC | DNA |
| | | encoding |
| | | gRNA |
| | | targeting |
| | | Arabidops |
| | | is PDS3 |
| 66 | GCTTCATGGCCGAAGATACG | DNA |
| | | encoding |
| | | gRNA |
| | | targeting |
| | | Arabidops |
| | | is ADH1 |
| 67 | GTTACAGGAGTAGTTCATCG | DNA |
| | | encoding |
| | | gRNA |
| | | targeting |
| | | Arabidops |
| | | is ACT8 |
| 68 | GGSGGGSG | Linker |
| 69 | (GGGGS)1-4 | Linker |
| 70 | AEAAAKEAAAKA | Linker |
| 71 | AEAAAKEAAAKEAAAKA | Linker |
| 72 | PAPAP (AP)6-8 | Linker |
| 73 | GIHGVPAA | Linker |
| 76 | EAAAK | |
| 77 | EAAAK EAAAK | |
| 78 | EAAAK EAAAK EAAAK | |
| 79 | EAAAK EAAAK EAAAK EAAAK | |
| 80 | GGAACTGACACACGACATGA | DNA |
| | | encoding |
| | | gRNA |
| | | targeting |
| | | Soybean |
| | | DD20 |
| 81 | ggccagtcacaatggctagtgtcattgcacggctacccaaaatattataccatct | mPing |
| | tctctcaaatgaaatcttttatgaaacaatccccacagtggaggggtttcttgaA | modified |
| | cgttccaagactaagcaaagcatttaattgatacaagttCgcgAAgaTtcatttg | with HSEs |
| | tacccaaaatccggcgcggcgcgggagaatgTTcTggAaggtcgcacggcggagg | |
| | cggacgcaagagatccggtgaatgTTCaagaatcggcctcaacgggggtttcact | |
| | ctgttaccgaggAacttTCTggaaacgacgctgacgagtttcaccaggatgaaac | |
| | tctttccAGAAAGttctctctcatccccatttcatgcaaataatcattttttatt | |
| | cagtcttacccctattaaatgtgcatgacacaccagtgaaacccccattgtgact | |
| | ggcc | |
| 82 | ttcttgaAcgttc | HSE1 |
| 83 | ttCgcgAAgaTtc | HSE2 |
| 84 | tTccAgAAcattc | HSE3 |
| 85 | ttcttGAAcattc | HSE4 |
| 86 | ttccAGAaagtTc | HSE5 |
| 87 | ttccAGAAAGttc | HSE6 |
| 88 | GGSGGSGGS | Linker |

SEQ ID NO: 74. All_in_one_vector: mPING in GFP, gRNA, Pong ORF1 and ORF2 linked to Cas9

1. An engineered nucleic acid modification system for generating a genetically modified soybean cell, the system comprising:
a. a donor polynucleotide comprising a first and second mPing miniature inverted-repeat transposable element (MITE) transposition sequences;
b. one or more nucleic acid constructs for expressing
i. a Pong ORF1 protein of a Pong transposase, the nucleic acid construct comprising a promoter operably linked to a nucleic acid sequence encoding the Pong ORF1 protein;
ii. a nucleic acid construct for expressing a targeting nuclease of a CRISPR/Cas system comprising a Cas nuclease and a guide RNA (gRNA), wherein the Cas nuclease is fused to the C-terminus of a Pong ORF2 protein of the Pong transposase by more than one copy of a flexible linker, the nucleic acid construct comprising a promoter operably linked to a nucleic acid sequence encoding the Cas nuclease fused to the C-terminus of the Pong ORF2 protein by more than one copy of a flexible linker; and
iii. a nucleic acid expression construct for expressing the gRNA of the CRISPR/Cas system;
wherein the CRISPR/Cas system is programmed to target the transposase and the donor polynucleotide to a target nucleic acid locus in the cell, to introduce a cut in the target nucleic acid locus, or both, thereby accomplishing insertion of the donor polynucleotide at the target nucleic acid locus to generate a genetically modified cell comprising the donor polynucleotide inserted at the target nucleic acid locus.

2. The engineered system of claim 1, wherein the first transposition sequence comprises a nucleic acid sequence comprising at least about 75% or more, at least about 85% or more, at least about 95% or more, or 100% sequence identity with SEQ ID NO: 7, SEQ ID NO: 111, or SEQ ID NO: 108 and wherein the second transposition sequence comprises a nucleic acid sequence comprising at least about 75% or more, at least about 85% or more, at least about 95% or more, or 100% sequence identity with SEQ ID NO: 8, SEQ ID NO: 112, or SEQ ID NO: 109.

3-4. (canceled)

5. The engineered system of claim 1, wherein the engineered system comprises an expression construct for expressing the Pong ORF1 protein and wherein the expression construct for expressing the Pong ORF1 protein comprises at least about 75% or more, at least about 85% or more, at least about 95% or more, or 100% sequence identity with SEQ ID NO: 100.

6-18. (canceled)

19. The engineered system of claim 1, wherein the Pong ORF2 protein is linked to the Cas9 nuclease by three copies of the flexible linker.

20. The engineered system of claim 19, wherein the Pong ORF2 protein is linked to the Cas9 nuclease by three copies of a G4S linker of SEQ ID NO: 64, wherein the Pong ORF2 protein is linked to the Cas9 nuclease by three copies of a G4S linker comprises an amino acid sequence encoded by a nucleic acid sequence comprising at least about 75% or more, at least about 85% or more, at least about 95% or more, or 100% sequence identity with a nucleic acid sequence of SEQ ID NO: 107.

21. The engineered system of claim 19, wherein the engineered system comprises an expression construct for expressing the Pong ORF2 protein linked to the Cas9 nuclease by three copies of a G4S linker of SEQ ID NO: 64, wherein the expression construct comprises a nucleic acid sequence comprising at least about 75% or more, at least about 85% or more, at least about 95% or more, or 100% sequence identity with SEQ ID NO: 104.

22-29. (canceled)

30. The engineered system of claim 1, wherein the first mPing transposition sequence and the second mPing transposition sequence flank a cargo polynucleotide and, wherein the cargo polynucleotide comprises an expression construct for expressing resistance to bialaphos herbicide.

31. (canceled)

32. The engineered system of claim 30, wherein the first mPing transposition sequence comprises a nucleic acid sequence comprising at least about 75% or more, at least about 85% or more, at least about 95% or more, or 100% sequence identity with SEQ ID NO: 108 and the second mPing transposition sequence comprises a nucleic acid sequence comprising at least about 75% or more, at least about 85% or more, at least about 95% or more, or 100% sequence identity with SEQ ID NO: 109.

33. The engineered system of claim 30, wherein the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97 or SEQ ID NO: 99.

34. The engineered system of claim 30, wherein the cargo polynucleotide comprises an expression construct comprising a promoter operably linked to a polynucleotide encoding a bialaphos resistance gene wherein the donor polynucleotide comprises a nucleic acid sequencing comprising at least about 75% or more, at least about 85% or more, at least about 95% or more, or 100% sequence identity with the nucleic acid sequence of SEQ ID NO: 97.

35-37. (canceled)

38. The engineered system of claim 1, wherein the engineered system comprises an expression construct for expressing a gRNA for targeting the transposase and nuclease to a target nucleic acid locus in a soybean DD20 intergenic region, wherein the expression construct for expressing a gRNA comprises a nucleic acid sequence comprising at least about 75% or more, at least about 85% or more, at least about 95% or more, or 100% sequence identity with the nucleic acid sequence of SEQ ID NO: 105.

39-57. (canceled)

58. A method of targeted insertion of a nucleic acid sequence into a target nucleic acid locus in a soybean cell, the method comprising:
a. introducing one or more nucleic acid constructs encoding an engineered nucleic acid modification system of claim 1 into the cell;
b. maintaining the cell under conditions and for a time sufficient for the donor polynucleotide to be inserted in the target locus; and
C. optionally identifying an insertion of the donor polynucleotide in the nucleic acid locus in the cell.

59-61. (canceled)

62. A kit for generating a genetically modified soybean cell, the kit comprising one or more engineered nucleic acid modification systems of claim 1, wherein each of the engineered systems generates an engineered soybean cell comprising an accurate insertion of the donor polynucleotide into the target nucleic acid locus.

63-65. (canceled)
